(19) United States
(12) Patent Application Publication
(10) Pub. No.: US 2018/0132516 A1
(43) Pub. Date: May 17, 2018

(54) POSITIVE ALLOSTERIC MODULATORS OF SWEET TASTE

(71) Applicant: Sensorygen, Inc., South Pasadena, CA (US)

(72) Inventors: Anandasankar RAY, Riverside, CA (US); Sean Michael BOYLE, Sunnyvale, CA (US)

(21) Appl. No.: 15/815,123

(22) Filed: Nov. 16, 2017

Related U.S. Application Data
(60) Provisional application No. 62/423,001, filed on Nov. 16, 2016.

(51) Int. Cl. A23L 27/00; A23L 2/60; C07J 9/00; C07J 17/00; A23L 27/30

(52) U.S. Cl. CPC A23L 27/88; A23L 2/60; A23L 27/30; C07J 17/005; C07J 9/00

(57) ABSTRACT

The invention provides natural allosteric modulators that are useful as sweet flavor enhancers. The present invention also includes ingestible compositions comprising the present compounds and methods of enhancing the sweet taste of sweeteners and sugars.

### CROSS-REFERENCE TO A RELATED APPLICATION

This application claims the priority benefit of U.S. Provisional Application Ser. No. 62/423,001, filed Nov. 16, 2016, which is incorporated herein by reference in its entirety.

### BACKGROUND OF INVENTION

Living things use sensory systems to detect important information about the environment in which they live. Taste is a particularly important sensory system, and is one of the most sophisticated forms of chemically-triggered sensations found throughout the animal kingdom.

Mammals are believed to have five basic taste modalities: sweet, bitter, sour, salty, and umami (savory). The pleasant taste associated with sweetness is important in industries that produce consumable materials. Natural sugars, such as sucrose, fructose, and glucose, for example, are heavily utilized by the beverage, food, pharmaceutical, and oral hygienic/cosmetic industries. However, many consumers are required to control or reduce the sugar and calorie content of their diets due to health concerns such as obesity, diabetes, and heart disease. Thus, there is a continuing need in the market to provide alternative, non-caloric or low-caloric sweeteners with sugar-like taste for consumers who need or desire to limit caloric intake.

One option for addressing this need is the use of natural high-potency sweeteners (NHPS). Because these sweeteners are many times sweeter than sucrose, for example, much less of the sweetener is required to replace the sugar; however, these sweeteners often possess taste qualities that vary from those of natural sugars. For example, certain sweetening compositions have different temporal profiles, maximal responses, flavor profiles, mouthfeel, and/or adaptation behaviors from those of sugar. In addition, high-potency sweeteners generally exhibit bitter, metallic, cooling, astringent, or licorice-like aftertastes, and/or diminishing sweetness on iterative tasting. These properties can be undesirable for consumers.

Therefore, improved compounds and methods for enhancing the sweetness of consumable materials, while retaining the characteristics of sugar and keeping calorie content low, are desired.

It is known that sensations associated with taste involve distinct signalling pathways mediated by receptors, which specifically recognize molecules that elicit those taste sensations. Recent developments in sweetening methods include the use of certain molecules that enhance the sweet taste of existing sweeteners and sugars, rather than acting as sweeteners themselves. The following disclosure provides new, natural positive modulator compounds for enhancing sweet taste.

### BRIEF SUMMARY

The present invention provides isolated compounds for enhancing sweet taste, ingestible compositions containing these compounds, and methods of preparing such ingestible compositions, to help meet the need for improved consumer-friendly, low-calorie sweetened products.

The present invention further provides improved methods of enhancing, modulating, or potentiating the perception, by a subject, of the sweet taste of an ingestible composition, such as a food, beverage, or pharmaceutical product.

Further provided are improved methods for modulating, particularly enhancing or potentiating, the activation of a sweet taste receptor.

Embodiments of the invention provide improved natural sweet taste modulators that enhance sweet taste receptor activity and sweetness perception while reducing the required concentration of, for example, an artificial sweetener or sugar. In certain embodiments, the sweet taste modulator is any one of the compounds from Table 1, or a combination thereof, or a salt or derivative thereof.

The sweet taste modulator can be combined with any suitable sweetener, such as, for example, a natural caloric sweetener; a natural high-potency sweetener; a synthetic sweetener, including synthetic high-potency sweeteners; sugar alcohols; rare sugars; sweetener enhancers; or combinations thereof, to provide an ingestible composition having enhanced sweetness.

In another embodiment, the present invention provides a method of increasing the sweet taste of an ingestible composition by adding a compound of the present invention to the ingestible composition. The compound used according to the current invention can be a chemosensory receptor modifier, a chemosensory receptor ligand modifier, or both, e.g., a partial chemosensory receptor modifier and partial chemosensory receptor ligand modifier. For example, the compound of the present invention can be a sweet receptor agonist, or a sweet enhancer, or a partial sweet receptor agonist and partial sweet enhancer.

In another embodiment, the present invention provides an ingestible composition comprising the sweet taste modulator and an ingestibly-acceptable excipient. According to particular embodiments, the ingestible composition can be a product selected from beverages, foods, pharmaceuticals, tobacco products, oral hygienic/cosmetics, nutraceuticals, and the like.

In yet another embodiment, the present invention provides a flavoring concentrate formulation comprising a compound of the present invention as a flavor modifying ingredient, a carrier, and optionally, at least one adjuvant.

### DETAILED DISCLOSURE

The present invention provides isolated compounds for enhancing sweet taste, ingestible compositions containing such compounds, and methods of preparing such ingestible compositions to help meet the need for improved low-calorie sweetened products.

The present invention further provides improved methods of enhancing, modulating, and/or potentiating the sweet taste of an ingestible composition, such as a food, beverage, or pharmaceutical product. The present invention also provides an improved method of modulating, particularly enhancing or potentiating, the activation of a sweet taste receptor.

The compounds of the invention include all stereochemical forms of the compound, including geometric isomers (i.e., E, Z) and optical isomers (i.e., R, S). Single stereochemical isomers as well as enantiomeric and diastereomeric mixtures of the present compounds are specifically contemplated. The compounds may also exist in several tautomeric forms. The term “tautomer” as used herein refers to isomers that change into one another with great ease such that they can exist together in equilibrium. Accordingly, the chemical structures depicted herein encompass all possible tautomeric forms of the illustrated compounds. Additionally, unless otherwise stated, formulas depicted herein are also meant to include compounds that differ only in the presence of one or more isotopically enriched atoms.

Further included within the scope of the present invention are various forms and derivatives of the exemplified compounds. Such forms include, but are not limited to, unsolvated, solvated, and hydrated forms. Further, derivative forms include, but are not limited to, acylated derivatives, alkylated derivatives, amidated derivatives, esterified derivatives, and other like derivatives. Furthermore, the compounds may exist in multiple crystalline or amorphous forms. In general, all physical forms are contemplated herein and are within the scope of the present invention.

### Selected Definitions

As used herein, an “ingestible composition” includes any substance that, either alone or together with another substance, can be ingested orally, whether intended for consumption or not. Ingestible compositions include, for example, “food or beverage products,” “pharmaceuticals,” as well as “non-edible products.” By “food or beverage products,” it is meant any edible product intended for consumption by humans or animals, including solids, semi-solids, and liquids (e.g., beverages). The term “non-edible products” or “non-comestible composition” includes any product or composition that can be taken orally by humans or animals for purposes other than consumption as food or beverage. For example, non-edible products and noncomestible compositions include pharmaceuticals and over the counter medications, oral care products such as dentifrices and mouthwashes, cosmetic products such as sweetened lip balms and other personal care products.

An “ingestibly acceptable carrier or excipient” is a medium and/or composition that is used to prepare a desired form of an inventive compound of the subject invention, in order to administer the compound. The medium and/or composition may be in any form depending on the intended use of a product, e.g., solid, semi-solid, liquid, paste, gel, cream, foamy material, suspension, solution, or any combination thereof (such as a liquid containing solids). Ingestibly acceptable carriers include many common food ingredients, such as water at neutral, acidic, or basic pH; fruit or vegetable juices; vinegar; marinades; beer, wine; natural water/fat emulsions such as milk or condensed milk; edible oils and shortenings; fatty acids and their alkyl esters; low molecular weight oligomers of propylene glycol; glyceryl esters of fatty acids; and dispersions or emulsions of such hydrophobic substances in aqueous media; salts such as sodium, calcium or potassium chloride; wheat flours; solvents such as ethanol; solid edible diluents such as vegetable powders or flours; other liquid vehicles; dispersion or suspension aids; surface active agents; isotonic agents; thickening or emulsifying agents; preservatives; solid binders; lubricants and the like.

According to the present invention, a “chemosensory receptor” can be any receptor associated with chemosensory sensation or chemosensory ligand triggered signal transduction, e.g., via taste receptors or taste-related receptors expressed in taste buds or internal organs of the body, such as in the gastrointestinal tract, etc. In one embodiment, a chemosensory receptor is a receptor that belongs to the 7-transmembrane receptor superfamily or G protein-coupled receptors (GPCRs). In another embodiment, a chemosensory receptor is a receptor carrying out signal transduction via one or more G proteins. In yet another embodiment, a chemosensory receptor is a receptor that belongs to family C or class C of GPCRs. In yet another embodiment, a chemosensory receptor is a receptor that belongs to the TIR family. In yet another embodiment, a chemosensory receptor is a receptor of TIR1, TIR2, TIR3, or their equivalents or variants or a combination thereof. In still another embodiment, a chemosensory receptor is a hetero-dimer of TAS1R2 and TAS1R3, or their equivalent or variant.

An “enhancer” refers to a compound that modulates (increases) the activation of a particular receptor, preferably a chemosensory, e.g., the TAS1R2/TAS1R3 receptor. Such enhancers typically enhance the activation of a chemosensory receptor by its ligand. Typically the “enhancer” will be specific to a particular ligand, i.e., it will not enhance the activation of a chemosensory receptor by chemosensory ligands other than the particular chemosensory ligand, or ligands closely related thereto.

Some enhancers, at their ligand-enhancing concentration, do not result in activation of the particular receptor by themselves. That is, the ligand-enhancing concentrations of these enhancers are concentration levels of the enhancers that increase or enhance the activation of a particular receptor by a ligand without substantially activating the particular receptor by the enhancers themselves. In some embodiments, certain enhancers, when used at a concentration higher than the ligand-enhancing concentration, can also activate a particular receptor by themselves in addition to modulating the activation of the receptor. For example, certain enhancers, when used at a concentration higher than the ligand-enhancing concentration, can be sweeteners (i.e., sweet flavoring agent/entity) as well. In other embodiments, certain enhancers can activate a particular receptor by themselves in addition to modulating the activation of the receptor simultaneously at the same concentration. In other words, certain enhancers are also sweeteners (i.e., sweet flavoring agent/entity) at the same time.

A “flavor” refers to the perception of taste in a subject, which includes sweet, sour, salty, bitter and umami. The subject may be a human or an animal.

A “flavoring agent” refers to a compound that induces a flavor or taste in an animal or a human. The flavoring agent can be natural, semi-synthetic, or synthetic.

A “flavor modifier” or “flavor modifying agent” refers to a compound that modulates, including enhancing or potentiating, and/or inducing, the tastes of a flavoring agent in an animal or a human.

A “flavor enhancer” refers to a compound that enhances the taste of a flavoring agent, or an ingestible composition comprising the flavoring agent.

A “sweet flavor” refers to the sweet taste typically induced by sugar, such as sucrose or fructose, in an animal or a human.

A “sweet flavoring agent,” “sweet flavor entity,” “sweetener,” or “sweet compound” refers to a compound that elicits a detectable sweet flavor in a subject, e.g., fructose or a compound that activates a TAS1R2/TAS1R3 receptor. The subject may be a human or an animal.

A “sweet flavor modifier” or “sweet flavor modifying agent” refers to a compound that modulates, including enhancing or potentiating, inducing, or blocking, the sweet taste of a sweet flavoring agent in an animal or a human. The sweet flavor modifier includes both sweet flavor enhancer and sweet flavoring agent.

A “sweet flavor enhancer” or “sweet flavor enhancing agent” refers to an enhancer of a sweet flavor.

A “sweet receptor activating compound” or “sweet receptor agonist” refers to a compound that activates a sweet receptor, such as, for example, a TAS1R2/TAS1R3 receptor. One example of a sweet receptor activating compound is a sweetener, such as sucrose or fructose.

A “sweet receptor modulating compound” refers to a compound that modulates (activates, blocks, or enhances/reduces activation of) a sweet receptor such as a TAS1R2/TAS1R3 receptor.

A “sweet receptor enhancing compound” refers to a compound that enhances or potentiates the effect of a sweet receptor activating compound.

A “sweet flavor modulating amount” refers to an amount of a compound that is sufficient to alter (either increase or decrease) sweet taste in an ingestible composition sufficiently to be perceived by a human subject. In many embodiments of the invention, at least about 0.001 ppm of the compound would need to be present in order for most human subjects to perceive a modulation of the sweet flavor of an ingestible composition comprising the compound. A broad range of concentrations that can be employed in order to provide a desirable degree of sweet flavor modulation can be from about 0.001 ppm to 100 ppm, or a narrow range from about 0.1 ppm to about 10 ppm. Alternative ranges of sweet flavor modulating amounts can be from about 0.01 ppm to about 30 ppm, from about 0.05 ppm to about 15 ppm, from about 0.1 ppm to about 5 ppm, or from about 0.1 ppm to about 3 ppm.

A “sweet flavor enhancing amount” refers to an amount of a compound that is sufficient to enhance the taste of flavoring agents, e.g., sucrose or fructose, in an ingestible composition, as perceived by an animal or a human. A broad range of a sweet flavor enhancing amount can be from about 0.001 ppm to 100 ppm, or a narrow range from about 0.1 ppm to about 10 ppm. Alternative ranges of sweet flavor enhancing amounts can be from about 0.01 ppm to about 30 ppm, from about 0.05 ppm to about 15 ppm, from about 0.1 ppm to about 5 ppm, or from about 0.1 ppm to about 3 ppm. In some embodiments, sweet flavor enhancing amount is the amount corresponding to ligand enhancing concentration(s) of a sweet flavor enhancer of the present invention.

A “sweet receptor modulating amount” refers to an amount of a compound that is sufficient to modulate (activate, enhance or block) a sweet taste receptor protein. In many embodiments of the invention, a sweet receptor modulating amount is at least about 10 nM, or at least about 100 nM, or at least about 1 μM, or at least about 10 μM.

A “TAS1R2/TAS1R3 receptor modulating or activating amount” is an amount of compound that is sufficient to modulate or activate a TAS1R2/TAS1R3 receptor.

A “sweet receptor” is a taste receptor that can be modulated by a sweet compound. Preferably a sweet receptor is a G protein coupled receptor, and more preferably the sweet receptor is a TAS1R2/TAS1R3 receptor.

The terms “isolated,” and “purified,” refer to material that is substantially or essentially free from components that normally accompany the compound as found in its native state. Purity and homogeneity are typically determined using analytical chemistry techniques. Particularly, in preferred embodiments, the compound is at least 85% pure, more preferably at least 90% pure, more preferably at least 95% pure, and most preferably at least 99% pure.

### Compounds of the Current Invention

In specific embodiments, the present invention provides sweet taste modulator compounds as shown in Table 1, as well as derivatives of the compounds exemplified in Table 1. In preferred embodiments, these compounds are isolated or purified.

The materials used in preparing the compounds of the invention, e.g., the various structural subclasses and species of the compounds or the synthetic precursors of the present compounds, are known compounds, or can be synthesized by known methods described in the literature, or are commercially available from various sources well known to those of ordinary skill in the art. These sources can be, for example, Sigma-Aldrich Corporation of St. Louis, Mo. USA and their subsidiaries Fluka and Riedel-de Haen, at their various other worldwide offices, and other well-known chemical suppliers such as Fisher Scientific, TCI America of Philadelphia, Pa., ChemDiv of San Diego, Calif., Chembridge of San Diego, Calif., Asinex of Moscow, Russia, SPECS/BIOSPECS of the Netherlands, Maybridge of Cornwall, England, Acros, TimTec of Russia, Comgenex of South San Francisco, Calif., and ASDI Biosciences of Newark, Del.

The skilled artisan in the art of organic chemistry can readily carry out the synthesis of many starting materials and subsequent manipulations without further direction. These include, for example, reduction of carbonyl compounds to their corresponding alcohols; oxidations; acylations; aromatic substitutions; both electrophilic and nucleophilic; etherifications; esterification; saponification; nitrations; hydrogenations; reductive animation and the like. These manipulations are discussed in standard texts such as March's Advanced Organic Chemistry (3d Edition, 1985, Wiley-Interscience, New York), Feiser and Feiser's Reagents for Organic Synthesis, and in the various volumes and editions or Methoden der Organischen Chemie (Houben-Weyl), and the like. Many general methods for preparation of materials comprising variously substituted heterocyclic, hetereoaryl, and aryl rings (the precursors of Ar, hAr1, and/or hAr2) can be found in Methoden der Organischen Chemie (Houben-Weyl), whose various volumes and editions are available from Georg Thieme Verlag, Stuttgart. The entire disclosures of the treatises recited above are hereby incorporated by reference in their entireties for their teachings regarding methods for synthesizing organic compounds and their precursors.

The skilled artisan will also readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts, Protecting Groups in Organic Synthesis, 3r Ed., John Wiley & Sons (1999).

### Use of the Compounds of the Subject Invention to Enhance Sweeteners

The compounds of Table 1, and derivatives thereof, can be used for one or more methods of the present invention. In one embodiment, the method can include increasing or enhancing sweet flavor. In another embodiment, the method can include modulating a sweet receptor and/or its ligand.

According to the present invention, a method of modulating a chemosensory receptor and/or its ligand can include modulating the activity, structure, function, expression, and/or modification of a chemosensory receptor as well as modulating, treating, or taking prophylactic measure of a condition, e.g., physiological or pathological condition, associated with a chemosensory receptor.

In general, the compounds of the present invention, individually or in combination, can be provided in a composition, such as, for example, an ingestible composition. In one embodiment, the compounds of the subject invention impart a more sugar-like temporal profile and/or flavor profile to a sweetener composition by combining one or more of the compounds with one or more sweeteners in a sweetener composition. In another embodiment, the compounds increase or enhance the sweet taste of a composition. In another embodiment, the compounds can be in a composition that modulates the sweet receptors and/or their ligands expressed at a place in the body other than in the taste buds.

The compounds or combination of compounds selected from Table 1 should preferably be comestibly acceptable, e.g., deemed suitable for consumption in food or drink, from the perspective of giving the comestible compositions an improved and/or pleasing sweet taste, and are not toxic and do not cause unpleasant or undesirable pharmacological or toxicological effects on a human or other animal at the typical concentrations they are employed as flavoring agents for the comestible compositions.

One of the methods of demonstrating that a flavorant compound is comestibly acceptable is to have the compound tested and/or evaluated by an Expert Panel of the Flavor and Extract Manufacturers Association (FEMA) and declared as to be “Generally Recognized As Safe” (“GRAS”). The FEMA/GRAS evaluation process for flavorant compounds is complex but well known to those of ordinary skill in the food product preparation arts, as is discussed by Smith, et al. in an article entitled “GRAS Flavoring Substances 21,” Food Technology, 57(5), pgs 46-59, May 2003, the entire contents of which are hereby incorporated herein by reference.

In addition to the FEMA expert panel, an independent, qualified panel of experts in pertinent scientific disciplines may be formed by the manufacturer to evaluate the safety of a specific compound for GRAS status. This process is known as a “self-determination of GRAS status.”

Another method of demonstrating that a flavorant compound is comestibly acceptable is to obtain favorable review by the WHO/FAO Joint Expert Committee on Food Additives, or JECFA. There are also other evaluation methods, such as independent review by the regulatory agency, which are generally known to those of ordinary skill in the food product preparation arts.

In one embodiment, the compounds of the present invention can be used at concentrations within acceptable oral toxicity levels. In another embodiment, the compounds of the present invention can be used at ligand enhancing concentrations, e.g., very low concentrations on the order of a few parts per million, in combination with one or more known sweeteners, natural or artificial, so as to reduce the concentration of the known sweetener required to prepare an ingestible composition having the desired degree of sweetness.

The compounds can be used to enhance the sweet taste or perception of sweet taste of any suitable caloric, low-caloric, or non-caloric sweetener. Non-limiting examples of such sweeteners include caloric carbohydrate sweeteners (including polyols), natural high-potency sweeteners, synthetic high-potency sweeteners, and combinations thereof.

As used herein, the phrase “natural high-potency sweetener” or “NHPS” means any sweetener found in nature, which may be in raw, extracted, purified, or any other form, singularly or in combination thereof and characteristically having a sweetness potency greater than sucrose, fructose, or glucose, but having fewer calories. Non-limiting examples of NHPSs include rebaudioside A, rebaudioside B, rebaudioside C, rebaudioside D, rebaudioside E, rebaudioside F, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I.

As used herein, the phrase “synthetic sweetener” or “synthetic high-potency sweetener” refers to any composition that is not found in nature and that has a sweetness potency greater than sucrose, fructose, or glucose, yet has fewer calories. Non-limiting examples of synthetic sweeteners include sucralose, acesulfame potassium or other salts, aspartame, alitame, saccharin, neohesperidin dihydrochalcone, cyclamate, neotame, N-[3-(3-hydroxy-4-methoxyphenyl)propyl]-L-α-aspartyl]-L-phenylalanine 1-methyl ester, N-[3-(3-hydroxy-4-methoxyphenyl)-3-methylbutyl]-L-α-aspartyl]-L-phenylalanine 1-methyl ester, N-[3-(3-methoxy-4-hydroxyphenyl)propyl]-L-α-aspartyl]-L-phenylalanine 1-methyl ester, salts thereof, and the like.

According to the present invention, NHPS and synthetic sweeteners can be used individually or in combination with other NHPSs and/or synthetic sweeteners. For example, the sweetener composition can comprise a single NHPS or a single synthetic sweetener; a single NHPS in combination with a single synthetic sweetener; one or more NHPSs in combination with a single synthetic sweeteners; a single NHPS in combination with one or more synthetic sweeteners; or one or more NHPSs in combination with one or more synthetic sweeteners. A plurality of NHPSs and/or synthetic sweeteners can be used so long as the combined effect does not adversely affect the taste of the sweetener composition or orally sweetened composition.

In one embodiment, the present compounds can enhance the sweetness of a sweetener under a broad range of pH, e.g., from acid pH to basic pH. The pH can be, for example, from about 2.5 to about 9.0; from about 2.5 to about 8.0; from about 2.8 to about 7.5; from about 3.0 to about 7, and from about 3.5 to about 7.

In certain embodiments, the present compounds can enhance the perceived sweetness of a fixed concentration of a sweetener in taste tests at a compound concentration of about 50 μM, 40 μM, 30 μM, 20 μM, or 10 μM at acidic to basic pH. In certain embodiments, the enhancement factor of the present compounds at the lower pH is substantially similar to the enhancement factor of the compounds at neutral pH.

Furthermore, in some embodiments, the present compounds have enhanced photostability. That is, when exposed to a light source, the compounds have stability and are less prone to degradation. Such photostability and consistent sweet enhancing property under a broad range of pH's render the compounds good candidates for use in a wide variety of foods and beverages.

Typically, at least a sweet receptor modulating amount, a sweet receptor ligand modulating amount, a sweet flavor modulating amount, a sweet flavoring agent amount, a sweet flavor enhancing amount, or a therapeutically effective amount of one or more of the present compounds will be added to the ingestible composition, optionally in the presence of known sweeteners, e.g., so that the sweet flavor modified ingestible composition has an increased sweet taste as compared to the ingestible composition prepared without the compounds of the present invention, as judged by human beings or animals in general, or in the case of formulations testing, as judged by a majority of a panel of at least eight human taste testers, via procedures commonly known in the field.

The concentration of sweet flavoring agent needed to modulate or improve the flavor of the ingestible composition will of course depend on many variables, including the specific type of the ingestible composition and its various other ingredients, especially the presence of other known sweet flavoring agents and the concentrations thereof, the natural genetic variability and individual preferences and health conditions of various human beings tasting the compositions, and the subjective effect of the particular compound on the taste of such chemosensory compounds.

One application of the present compounds is for modulating (inducing, enhancing or inhibiting) the sweet taste or other taste properties of natural or synthetic sweeteners, and ingestible compositions made therefrom. In one embodiment, the compounds of the present invention are used or provided in their ligand enhancing concentration(s).

In one embodiment, the present invention provides a sweet enhancing composition. The sweet enhancing composition comprises a compound of the present invention in an amount effective to enhance sweetening, e.g., sweet flavor enhancing amount in combination with a first amount of sweetener, wherein the sweetening is more than the sweetening provided by the first amount of sweetener without the compound of the subject invention.

In one embodiment, the present invention provides an ingestible composition that comprises the sweet enhancing composition of the present invention. In certain embodiments, the ingestible composition is in the form of a food or beverage product, a pharmaceutical composition, a nutritional product, a dietary supplement, over-the-counter medication, or oral care product.

### Noncomestible Products

In certain embodiments, a compound of the present invention is added to a noncomestible composition or non-edible product, such as a pharmaceutical product, over the counter (OTC) product, oral care product, cosmetic product such as sweetened lip balms, or other personal care product.

Over the counter (OTC) product and oral care product refer to products for household and/or personal use that may be sold without a prescription and/or without a visit to a medical professional. Examples of OTC products include, but are not limited to, vitamins and dietary supplements; topical analgesics and/or anesthetics; cough, cold and allergy remedies; antihistamines and/or allergy remedies; and combinations thereof.

Vitamins and dietary supplements include, but are not limited to, vitamins, dietary supplements, tonics/bottled nutritive drinks, child-specific vitamins, dietary supplements, and any other such products.

Topical analgesics and/or anesthetics include any topical creams/ointments/gels used to alleviate superficial or deep-seated aches and pains, e.g., muscle pain; teething gel; patches with analgesic ingredient; and combinations thereof.

Cough, cold and allergy remedies include, but are not limited to, decongestants; cough remedies; pharyngeal preparations; medicated confectioneries; antihistamines and child-specific cough, cold and allergy remedies; and combination products.

Antihistamines and/or allergy remedies include, but are not limited to, any systemic treatments for hay fever, nasal allergies, and insect bites and stings.

Examples of oral care products include, but are not limited to mouth cleaning strips, toothpaste, toothbrushes, mouthwashes/dental rinses, denture care, mouth fresheners at-home teeth whiteners, dentifrices, and dental floss.

### Food and Beverage Products

In another embodiment, the present compounds are added to food or beverage products or formulations. Examples of food and beverage products or formulations include, but are not limited to sweet coatings, frostings, or glazes for comestible products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionary category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid that may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consomme) to sauces (cream or cheese-based soups).

The Dehydrated and Culinary Food Category means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes alcoholic drinks, soft drinks, sports drinks, isotonic beverages, and hot drinks. Alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water, functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category refers to any food that can be a light informal meal including, but not limited to, sweet and savory snacks and snack bars. Examples of snack food include, but are not limited to, fruit snacks, chips/crisps, extruded snacks, tortilla/corn chips, popcorn, pretzels, nuts and other sweet and savory snacks. Examples of snack bars include, but are not limited to, granola/muesli bars, breakfast bars, energy bars, fruit bars and other snack bars.

The Baked Goods category refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionary category refers to edible product that is sweet to the taste. Examples of confectionary include, but are not limited to, candies, gelatins, chocolate confectionery, sugar confectionery, gum, and the likes and any combination products.

The Meal Replacement category refers to any food intended to replace the normal meals, particularly for people having health or fitness concerns. Examples of meal replacement include, but are not limited to, slimming products and convalescence products.

The Ready Meal category refers to any food that can be served as a meal without extensive preparation or processing. The ready meal includes products that have had recipe “skills” added to them by the manufacturer, resulting in a high degree of readiness, completion and convenience. Examples of ready meals include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to, canned/preserved meat and meat products, fish/seafood, vegetables, tomatoes, beans, fruit, ready meals, soup, pasta, and other canned/preserved foods.

The Frozen Processed Food category includes, but is not limited to, frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to, rice, dessert mixes, dried ready meals, dehydrated soup, instant soup, dried pasta, plain noodles, and instant noodles. The Chill Processed Food category includes, but is not limited to, chilled processed meats, processed fish/seafood products, lunch kits, fresh cut fruits, ready meals, pizza, prepared salads, soup, fresh pasta and noodles.

The Sauces, Dressings and Condiments category includes, but is not limited to, tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to, milk- or soybean-based formula; and prepared, dried and other baby food.

The Spreads category includes, but is not limited to, jams and preserves, honey, chocolate spreads, nut based spreads, and yeast based spreads.

The Dairy Product category generally refers to edible product produced from mammal's milk. Examples of dairy product include, but are not limited to, drinking milk products, cheese, yoghurt and sour milk drinks, and other dairy products.

Additional examples for comestible composition, particularly food and beverage products or formulations, are provided as follows. Exemplary comestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, gum, chewing gum, sugarized gum, sugar-free gum, functional gum, bubble gum, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary comestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary comestible compositions also include breakfast cereals, sweet beverages or solid or liquid concentrate compositions for preparing beverages, ideally so as to enable the reduction in concentration of previously known saccharide sweeteners, or artificial sweeteners.

### Concentrates

According to another aspect of the invention, the compounds of the present invention can be provided in a concentrate formulation, e.g., suitable for subsequent processing to produce a ready-to-use (i.e., ready-to-serve) product.

By “a flavoring concentrate formulation,” is meant a formulation that can be reconstituted with one or more diluting media to become a ready-to-use composition. The term “ready-to-use composition” is used interchangeably herein with “ingestible composition,” which denotes any substance that, either alone or together with another substance, can be taken orally whether intended for consumption or not. In one embodiment, the ready-to-use composition includes a composition that can be directly consumed by a human or animal.

The flavoring concentrate formulation is typically used by mixing with one or more diluting media, e.g., any consumable or ingestible ingredient or product, to impart or modify one or more flavors to the diluting medium. Such a process is often referred to as reconstitution.

The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by, for example, a restaurant, vendor, or a manufacturer in large industrial scale to produce the ready-to-use soft drinks.

Because the flavoring concentrate formulation has the flavoring agent or flavor modifying agent in a concentration higher than the ready-to-use composition, the flavoring concentrate formulation is typically not suitable for being consumed directly without reconstitution.

There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

In one embodiment, the flavoring concentrate formulation comprises a compound of the present invention; a carrier, and optionally, at least one adjuvant. The term “as flavor modifying ingredient” denotes that the compound of the present invention acts as a flavoring agent or a flavor modifying agent (such as a flavor enhancer) in the formulation. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive that supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described herein. In another embodiment, the at least one adjuvant comprises one or more ingredients selected from emulsifiers, stabilizers, antimicrobial preservatives, antioxidants, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, which is hereby incorporated by reference in its entirety.

The present flavoring concentrate formulation can be in a form selected from, for example, liquid including solution and suspension, solid, foamy material, paste, gel, cream, and a combination thereof, such as a liquid containing a certain amount of solid contents. In one embodiment, the flavoring concentrate formulation is in the form of a liquid, including aqueous-based and non-aqueous-based. The flavoring concentrate formulation can be carbonated or non-carbonated.

The flavoring concentrate formulation may further comprise a freezing point depressant, nucleating agent, or both as the at least one adjuvant. The freezing point depressant is a ingestibly acceptable compound or agent that can depress the freezing point of a liquid or solvent to which the compound or agent is added. That is, a liquid or solution containing the freezing point depressant has a lower freezing point than the liquid or solvent without the freezing point depressant. In addition to depressing the onset freezing point, the freezing point depressant may also lower the water activity of the flavoring concentrate formulation. Examples of freezing point depressants include, but are not limited to, carbohydrates, oils, ethyl alcohol, polyol, e.g., glycerol, and combinations thereof.

Nucleating agents are ingestibly acceptable compounds or agents that facilitate nucleation. The presence of a nucleating agent in the flavoring concentrate formulation can improve the mouthfeel of, for example, a frozen slush and help maintain the physical properties and performance of the slush at freezing temperatures by increasing the number of ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the concentrate formulation has a water activity of less than about 0.85. In another embodiment, the concentrate formulation has a water activity of less than about 0.80. In another embodiment, the concentrate formulation has a water activity of less than about 0.75.

In one embodiment, the flavoring concentrate formulation has the present compound in a concentration that is at least 2, 5, 10, 20, 30, 40, 50, 75, 100, or more times the concentration of the compound in a ready-to-use composition.

Having now generally described the invention, the same will be more readily understood by reference to the following examples, which are provided by way of illustration and are not intended as limiting. It is understood that various modifications and changes can be made to the herein disclosed exemplary embodiments without departing from the spirit and scope of the invention.

### EXAMPLES

### Example 1—General Method for Measuring Taste Receptor Activity —Fluo-4 Calcium Assay

Fluo-4 is a fluorescent indicator for intracellular calcium and allows the determination of changes in calcium concentration, in particular an increase in response to receptor activation occurring after ligand addition. HEK293 cells stably expressing Gα16-gustducin 44 (Gα16gust44) were used as host cells and transfected with various constructs as described in examples 2-5. Black, clear-bottom 96-well plates were used for all assays. They were seeded the day before the assay with 8500 transfected cells per well and maintained at 37° C. overnight in a growth medium appropriate for the cells used. For HEK293 cells, Dulbecco's Modified Eagle medium supplemented with 10% fetal bovine serum was used for growth and maintenance of the HEK293 cells.

At the time of the assay, the growth medium was discarded and cells were incubated for 1 hour (at 37° C. in the dark) with 50 μl of a calcium assay solution consisting of 1.5 μM Fluo-4 AM (Molecular Probes™, Invitrogen, US) and 2.5 μM probenicid (Sigma-Aldrich) dissolved in a C1 buffer solution. C1 buffer solution contains 130 mM NaCl, 5 mM KCl, 10 mM Hepes, 2 mM CaCl₂ and 10 mM glucose (pH 7.4).

After the initial 1 hour loading period, the plates were washed 5 times with 100 μl per well of C1 buffer using an automated plate washer (BioTek) and after washing, the cells were further incubated in 100 μl C1 buffer per well for 30 minutes at room temperature in the dark to allow for complete de-esterification of the Fluo-4-AM. The buffer solutions were discarded, the plate was washed 5 times with 100 μl C wash buffer and finally the cells were placed in 180 μl of C1 wash buffer.

For assay reading, the plate was placed in a FLIPR (fluorescence imaging plate reader (FLIPR-Tetra, Molecular Devices), and receptor activation was initiated following addition of 20 μl of a 10× concentrated ligand stock solution. Fluorescence was continuously monitored for 15 seconds prior to ligand addition and for 105 seconds after ligand addition (45-105 sec may be sufficient).

Receptor activation is given in relative fluorescence units (RFU) and is defined by the following equation: Fluorescence Increase—Maximum Fluorescence—baseline fluorescence, wherein the baseline fluorescence represents the mean fluorescence calculated for the first 10 to 15 seconds prior to ligand addition.

Alternatively, Receptor activation is determined by the increase in peak fluorescence (F) normalized to the baseline fluorescence (F₀). The data are normalized using the following equation: ΔF/F=(F−F₀)/F₀, where F is the peak fluorescence signal and F₀ is the baseline fluorescence signal, wherein the baseline fluorescence represents the mean fluorescence calculated for the first 10 to 15 seconds prior to ligand addition.

As a negative control, mock transfected cells were exposed to the same concentration of ligand and the concentration of calcium traces not corresponding to a signal was determined. Cells with an activated receptor were identified by the signal (RFU or ΔF/F) being significantly above the negative control.

### Example 2—Sweet Flavor and Sweet Flavor Enhancement Measurement Using Human Panelists Conducting a Paired Comparison Test

Test samples containing experimental compounds are presented in pairs to the panelist and they are asked to determine which of the samples is sweeter. The present compounds can be tested for sweet flavor enhancement in a medium with a wide range of pH values, for example, at pH of about 7.1 or about 2.8. A group of 10 to 16 or more panelists can participate in each test. Subjects refrain from eating or drinking (except water) for at least 1 hour prior to the test. Subjects rinse with water several times to clean the mouth prior to testing.

Taste tests were performed with sucrose or HFCS as the sweetener in the presence or absence of compound. A 0.2% stock solution of compound in water with sodium bicarbonate was prepared and then this stock solution was diluted in the final sample to achieve the targeted final concentration of compound. Taste samples were also prepared in a low sodium phosphate buffer (pH 7.1; “LSB”) lacking sucrose or HFCS to evaluate the taste of the compound alone.

Low sodium phosphate buffer consists of 0.3 mM KCl, 0.5 mM Na2HPO4, and 0.175 mM KH2PO4. Sample volumes are usually 20 ml.

In one paired comparison test, the panelist is presented with two different samples and asked to identify the sample which is sweeter. The samples within a paired comparison test are presented in a randomized, counterbalanced order. Panelists have up to a 1 minute delay between taste tests to clear the mouth of any tastes.

Binomial probability tables are used to determine the probability of the correct number of responses occurring for each test at alpha=0.05.

### Example 3—Ranking Tests to Determine the Sweetness Isointensity of Sweetness Enhancers

For comparative ranking, samples of 0.5%, 1%, 1.5%, 7%, 8%, 9%, 10% and 11% sucrose solutions can be prepared.

### a) Sweetness Isointensity of Sweetness Enhancer in Sucrose Solutions

The sensory evaluation can be conducted using a ranking method. Samples at ambient temperature can be randomly presented in 15 ml blind aliquots (unidentifiable by panelists). Panels consisting of 10 sweet sensitive subjects and samples can be presented in 3 replications over 1 session. After tasting each sample, the mouth is rinsed thoroughly with water at ambient temperature prior to tasting the next sample. Panelists can be presented with 7%, 8%, 9%, 10%, 11% sucrose samples and a sixth sample of 7% sucrose with a sweetness enhancer in a concentration near its sweetness detection threshold. They can be asked to rank the samples from low to high with respect to perceived sweet taste. R-indices can be calculated for 7% sucrose with the sweetness enhancer versus 7%, 8%, 9%, 10% or 11% sucrose.

### b) Near Threshold Sweetness Isointensity of Sweetness Enhancer in Water

The sensory evaluation can be conducted using a ranking method. Samples at ambient temperature can be randomly presented in 15 ml blind aliquots (unidentifiable by panelists). Panels consisting of 10 sweet sensitive subjects and samples can be presented in 3 replications over 1 session. After tasting each sample, the mouth is rinsed thoroughly with water at ambient temperature prior to tasting the next sample.

Panelists can be presented with either 0.5% and 1% sucrose or 1% and 1.5% sucrose and a third sample of water with a sweetness enhancer in a concentration near its sweetness detection threshold. They can be asked to rank the samples from low to high with respect to perceived sweet taste.

R-indices can be calculated for the sweetness enhancer in water versus either 0.5% and 1% sucrose or 1% and 1.5% sucrose. The R-index is a statistic obtained by an analytical procedure based on signal detection and is a short-cut method for determining the proportion of subjects choosing one sample over another (see O'Mahony, 1992, J. Sens. Stud., 7:1-47). From ranking-style sensory tests, a matrix of responses (see table 2 below) is constructed such that each cell contains the number of times a given sample was ranked at a particular location.

TABLE 2

| | Position 1 | Position 2 | |
|---|---|---|---|
| Sample | (least sweet) | (most sweet) | Position 3 |
| Sample X | A | B | C |
| Sample Y | D | E | F |
| Sample Z | G | H | I |

From this matrix, the R-index can be calculated according to the following equation:

R−index(X vs. Y)=A(E+F)+B(F)+0.5((A*D)+(B*E)+(C*F))(A+B+C)*(D+E+F).

Essentially, the R-index is a measure of difference between two samples. The R-index has a chance level of detection of 50%. Thus, when comparing two samples, the derived R-index must be significantly different from 50% in order for them to be considered different. The critical value is the statistically relevant value that the derived R-index must deviate from (i.e., greater or less than) in order to be considered significant.

An R-index greater than the higher critical value means that the sweetness enhancer sample is significantly sweeter than the sucrose sample. An R-index not significantly different from the critical value means that the sweetness enhancer sample has an equivalent sweetness to the compared sucrose sample. An R-index below the lower critical value indicates that the sucrose sample is sweeter than the sweetness enhancer sample.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

TABLE 1

| | NAME (if | Present |
|---|---|---|
| SMILES STRUCTURE | available) | In |
| CC(C)CC(═O)OCCC(C)CCCC(C)═C | Rhodinyl | |
| | isovalerate | |
| CCC(═O)OC\C═C(\C)CCC═C(C)C | Neryl propio-te | FooDB |
| CCCCCCCCCCCCC1CCC(═O)O1 | 5-Dodecyldihydro- | FooDB |
| | 2(3H)-furanone | |
| OCC(O)C(O)CO | D-Threitol | FooDB |
| NC1═NC═NC2═C1N═CN2[C@@H]1O[C@@H]2COP(O)(═O)O[C@H]2 | Cyclic AMP | FooDB |
| [C@H]1O | | |
| OC[C@H]1O[C@H](O)[C@H](O)[C@@H](O)[C@H]1O | D-Galactose | FooDB |
| O1C═CC═C1C1═CC═CS1 | 2-(2-Thienyl)furan | FooDB |
| COC1═CC2═C(NC═C2CCNC(C)═O)C═C1 | Melatonin | FooDB |
| [O−]P([O−])(═O)OP([O−])([O−])═O | Pyrophosphate | FooDB |
| C\C(CCC═C)═C/CC[C@](C)(O)C═C | (3S,6E)-Nerolidol | FooDB |
| CCCC\C═C/CCOC(═O)CC | cis-3-Octenyl | FooDB |
| | propio-te | |
| CC1═COC2═C1C(═O)C1C(C)(O)CCC(O)C1(C)C2 | Curcolonol | FooDB |
| CN(C)C(═N)NCCC[C@H](N)C(O)═O | \NG,NG-Dimethyl- | FooDB |
| | L-arginine\ | |
| [H]OC1([H])C([H])([H])OC(═O)C([H])([H])C([H])([H])C1([H])O[H] | Solerol | FooDB |
| OC[C@@]1(O)CO[C@@H](OC[C@H]2O[C@@H](OC3═CC4═C(C(O)═ | Apigenin 7-apiosyl- | FooDB |
| C3)C(═O)C═C(O4)C3═CC═C(O)C═C3)[C@H](O)[C@@H](O)[C@@H]2 | glucoside | |
| O)[C@@H]1O | | |
| [H]OC([H])(C(═O)N([H])C([H])([H])C([H])([H])C(═O)N([H])C([H])([H])C([H]) | \3a,7a,12a,24- | FooDB |
| ([H])SC(═O)C([H])(C([H])([H])[H])C([H])(O[H])C([H])([H])C([H])([H])C([H]) | Tetrahydroxy-5b- | |
| (C([H])([H])[H])C1([H])C([H])([H])C([H])([H])C2([H])C3([H])[C@]([H])(O[H]) | cholestanoyl-CoA\ | |
| C([H])([H])[C@]4([H])C([H])([H])[C@]([H])(O[H])C([H])([H])C([H])([H])C4 | | |
| (C([H])([H])[H])C3([H])C([H])([H])[C@]([H])(O[H])C12C([H])([H])[H])C(C([H]) | | |
| ([H])[H])(C([H])([H])[H])C([H])([H])OP(═O)(O[H])OP(═O)(O[H])OC([H]) | | |
| ([H])[C@@]1([H])O[C@@]([H])(N2C([H])═NC3═C2N═C([H])N═C3N([H])[H]) | | |
| [C@]([H])(O[H])[C@]1([H])OP(═O)(O[H])O[H] | | |
| [H]OC([H])(C(═O)N([H])C([H])([H])C([H])([H])C(═O)N([H])C([H])([H])C([H]) | \3a,7a,12a- | FooDB |
| ([H])SC(═O)C([H])(C(═O)C([H])([H])C([H])([H])C([H])(C([H])([H])[H])C1 | Trihydroxy-5b-24- | |
| ([H])C([H])([H])C([H])([H])[C@@]2([H])C3([H])[C@]([H])(O[H])C([H])([H])[C | oxocholestanoyl- | |
| @]4([H])C([H])([H])[C@]([H])(O[H])C([H])([H])C([H])([H])C4(C([H])([H])[H]) | CoA\ | |
| [C@@]3([H])C([H])([H])[C@]([H])(O[H])C12C([H])([H])[H])C([H])([H])[H]) | | |
| C(C([H])([H])[H])(C([H])([H])[H])C([H])([H])OP(═O)(O[H])OP(═O)(O[H])OC | | |
| ([H])([H])[C@@]1([H])O[C@@]([H])(N2C([H])═NC3═C2N═C([H])N═C3N | | |
| ([H])[H])[C@]([H])(O[H])[C@]1([H])OP(═O)(O[H])O[H] | | |
| [H]O[C@]1([H])C([H])([H])C([H])([H])[C@@]2(C([H])([H])[H])[C@@]([H]) | \3a,7a-Dihydroxy- | FooDB |
| (C1([H])[H])C([H])([H])[C@@]([H])(O[H])[C@@]1([H])[C@]3([H])C([H])([H]) | 5b-cholestane\ | |
| C([H])([H])[C@]([H])([C@]([H])(C([H])([H])[H])C([H])([H])C([H])([H])C([H]) | | |
| ([H])C([H])(C([H])([H])[H])C([H])([H])[H])[C@@]3(C([H])([H])[H])C([H])([H]) | | |
| C([H])([H])[C@]21[H] | | |
| [H]O[C@]1([H])C([H])([H])C([H])([H])[C@@]2(C([H])([H])[H])C([H])(C1([H]) | \3a,7a-Dihydroxy- | FooDB |
| [H])C([H])([H])[C@@]([H])(O[H])C1([H])C3([H])C([H])([H])C([H])([H])C | 5b-cholestan-26-al\ | |
| ([H])(C([H])(C([H])([H])[H])C([H])([H])C([H])([H])C([H])([H])C([H])(C([H])═O) | | |
| C([H])([H])[H])[C@@]3(C([H])([H])[H])C([H])([H])C([H])([H])C21[H] | | |
| [H]OC(═O)C([H])([H])N([H])C(═O)C([H])(N([H])[H])C([H])([H])[H] | Alanylglycine | FooDB |
| [H]OC([H])(C(═O)N([H])C([H])([H])C([H])([H])C(═O)N([H])C([H])([H])C([H]) | \3a,7a-Dihydroxy- | FooDB |
| ([H])SC(═O)C(═C([H])C([H])([H])C([H])([H])C([H])(C([H])([H])[H])C1([H]) | 5b-cholest-24- | |
| C([H])([H])C([H])([H])C2([H])C3([H])[C@]([H])(O[H])C([H])([H])[C@]4([H]) | enoyl-CoA\ | |
| C([H])([H])[C@]([H])(O[H])C([H])([H])C([H])([H])[C@]4(C([H])([H])[H])C3 | | |
| ([H])C([H])([H])C([H])([H])[C@]12C([H])([H])[H])C([H])([H])[H])C(C([H])([H]) | | |
| [H])(C([H])([H])[H])C([H])([H])OP(═O)(O[H])OP(═O)(O[H])OC([H])([H])[C@ | | |
| @]1([H])O[C@@]([H])(N2C([H])═NC3═C2N═C([H])N═C3N([H])[H])[C@] | | |
| ([H])(O[H])[C@]1([H])OP(═O)(O[H])O[H] | | |
| [H]OC([H])(C(═O)N([H])C([H])([H])C([H])([H])C(═O)N([H])C([H])([H])C([H]) | \3a,7a-Dihydroxy- | FooDB |
| ([H])SC(═O)C([H])(C(═O)C([H])([H])C([H])([H])C([H])(C([H])([H])[H])C1([H]) | 5b-24- | |
| C([H])([H])C([H])([H])C2([H])C3([H])[C@]([H])(O[H])C([H])([H])[C@]4 | oxocholestanoyl- | |
| ([H])C([H])([H])[C@]([H])(O[H])C([H])([H])C([H])([H])[C@]4(C([H])([H])[H])C | CoA\ | |
| 3([H])C([H])([H])C([H])([H])[C@]12C([H])([H])[H])C([H])([H])[H])C(C([H]) | | |
| ([H])[H])(C([H])([H])[H])C([H])([H])OP(═O)(O[H])OP(═O)(O[H])OC([H])([H]) | | |
| [C@@]1([H])O[C@@]([H])(N2C([H])═NC3═C2N═C([H])N═C3N([H])[H])[C | | |
| @]([H])(O[H])[C@]1([H])OP(═O)(O[H])O[H] | | |
| [H]OC1([H])[C@@]([H])(O[C@]([H])(C([H])([H])OP(═O)(O[H])OP(═O)(O | \CDP- | FooDB |
| [H])OC([H])([H])[C@]([H])(OC(═O)C([H])([H])C([H])([H])C([H])([H])C([H]) | DG(16:0/18:2(9Z,12Z))\ | |
| ([H])C([H])([H])C([H])([H])C([H])([H])C([H])═C([H])C([H])([H])C([H])═C([H])C | | |
| ([H])([H])C([H])([H])C([H])([H])C([H])([H])C([H])([H])[H])C([H])([H])OC(═O) | | |
| C([H])([H])C([H])([H])C([H])([H])C([H])([H])C([H])([H])C([H])([H])C([H])([H]) | | |
| C([H])([H])C([H])([H])C([H])([H])C([H])([H])C([H])([H])C([H])([H])C([H])([H]) | | |
| C([H])([H])[H])[C@]1([H])O[H])N1C([H])═C([H])C(═NC1═O)N([H])[H] | | |
| [H]OC1([H])[C@@]([H])(O[C@]([H])(C([H])([H])OP(═O)(O[H])OP(═O)(O | \CDP- | FooDB |
| [H])OC([H])([H])[C@]([H])(OC(═O)C([H])([H])C([H])([H])C([H])([H])C([H])═ | DG(16:0/20:4(5Z,8Z, | |
| C([H])C([H])([H])C([H])═C([H])C([H])([H])C([H])═C([H])C([H])([H])C([H])═C | 11Z,14Z))\ | |
| ([H])C([H])([H])C([H])([H])C([H])([H])C([H])([H])C([H])([H])[H])C([H])([H])O | | |
| C(═O)C([H])([H])C([H])([H])C([H])([H])C([H])([H])C([H])([H])C([H])([H])C | | |
| ([H])([H])C([H])([H])C([H])([H])C([H])([H])C([H])([H])C([H])([H])C([H])([H])C | | |
| ([H])([H])C([H])([H])[H])[C@]1([H])O[H])N1C([H])═C([H])C(═NC1═O)N([H]) | | |
| [H] | | |
| [H]OC1([H])[C@@]([H])(O[C@]([H])(C([H])([H])OP(═O)(O[H])OP(═O)(O | \CDP- | FooDB |
| [H])OC([H])([H])[C@]([H])(OC(═O)C([H])([H])C([H])([H])C([H])([H])C([H]) | DG(16:0/20:4(8Z,11Z, | |
| ([H])C([H])([H])C([H])([H])C([H])═C([H])C([H])([H])C([H])═C([H])C([H])([H])C | 14Z,17Z))\ | |
| ([H])═C([H])C([H])([H])C([H])═C([H])C([H])([H])C([H])([H])[H])C([H])([H])O | | |
| C(═O)C([H])([H])C([H])([H])C([H])([H])C([H])([H])C([H])([H])C([H])([H])C | | |
| ([H])([H])C([H])([H])C([H])([H])C([H])([H])C([H])([H])C([H])([H])C([H])([H])C | | |
| ([H])([H])C([H])([H])[H])[C@]1([H])O[H])N1C([H])═C([H])C(═NC1═O)N([H]) | | |
| [H] | | |
| [H]OC1([H])[C@@]([H])(O[C@]([H])(C([H])([H])OP(═O)(O[H])OP(═O)(O | \CDP- | FooDB |
| [H])OC([H])([H])[C@]([H])(OC(═O)C([H])([H])C([H])([H])C([H])([H])C([H]) | DG(18:0/18:2(9Z,12Z))\ | |
| ([H])C([H])([H])C([H])([H])C([H])([H])C([H])═C([H])C([H])([H])C([H])═C([H])C | | |
| ([H])([H])C([H])([H])C([H])([H])C([H])([H])C([H])([H])[H])C([H])([H])OC(═O) | | |
| C([H])([H])C([H])([H])C([H])([H])C([H])([H])C([H])([H])C([H])([H])C([H])([H]) | | |
| C([H])([H])C([H])([H])C([H])([H])C([H])([H])C([H])([H])C([H])([H])C([H])([H]) | | |
| C([H])([H])C([H])([H])C([H])([H])[H])[C@]1([H])O[H])N1C([H])═C([H])C(═N | | |
| C1═O)N([H])[H] | | |
| [H]OC1([H])[C@@]([H])(O[C@]([H])(C([H])([H])OP(═O)(O[H])OP(═O)(O | \CDP- | FooDB |
| [H])OC([H])([H])[C@]([H])(OC(═O)C([H])([H])C([H])([H])C([H])([H])C([H])═ | DG(18:0/20:4(5Z,8Z, | |
| C([H])C([H])([H])C([H])═C([H])C([H])([H])C([H])═C([H])C([H])([H])C([H])═C | 11Z,14Z))\ | |
| ([H])C([H])([H])C([H])([H])C([H])([H])C([H])([H])C([H])([H])[H])C([H])([H])O | | |
| C(═O)C([H])([H])C([H])([H])C([H])([H])C([H])([H])C([H])([H])C([H])([H])C | | |
| ([H])([H])C([H])([H])C([H])([H])C([H])([H])C([H])([H])C([H])([H])C([H])([H])C | | |
| ([H])([H])C([H])([H])C([H])([H])C([H])([H])[H])[C@]1([H])O[H])N1C([H])═C | | |
| ([H])C(═NC1═O)N([H])[H] | | |
| [H]OC(═O)C1═C([H])N═C(C(O[H])═C1C(═O)O[H])C([H])([H])[H] | \3-Hydroxy-2- | FooDB |
| | methylpyridine-4,5- | |
| | dicarboxylate\ | |
| [H]OC1([H])[C@@]([H])(O[C@]([H])(C([H])([H])OP(═O)(O[H])OP(═O)(O | \CDP- | FooDB |
| [H])OC([H])([H])[C@]([H])(OC(═O)C([H])([H])C([H])([H])C([H])([H])C([H]) | DG(16:0/22:3(10Z, | |
| ([H])C([H])([H])C([H])([H])C([H])([H])C([H])([H])C([H])═C([H])C([H])([H])C([H])═ | 13Z,16Z))\ | |
| C([H])C([H])([H])C([H])═C([H])C([H])([H])C([H])([H])C([H])([H])C([H])([H]) | | |
| C([H])([H])[H])C([H])([H])OC(═O)C([H])([H])C([H])([H])C([H])([H])C([H])([H]) | | |
| C([H])([H])C([H])([H])C([H])([H])C([H])([H])C([H])([H])C([H])([H])C([H]) | | |
| ([H])C([H])([H])C([H])([H])C([H])([H])C([H])([H])[H])[C@]1([H])O[H])N1C([H])═ | | |
| C([H])C(═NC1═O)N([H])[H] | | |
| [H]OC1([H])[C@@]([H])(O[C@]([H])(C([H])([H])OP(═O)(O[H])OP(═O)(O | \CDP- | FooDB |
| [H])OC([H])([H])[C@]([H])(OC(═O)C([H])([H])C([H])([H])C([H])═C([H])C([H]) | DG(16:0/22:6(4Z,7Z, | |
| ([H])C([H])═C([H])C([H])([H])C([H])═C([H])C([H])([H])C([H])═C([H])C([H]) | 10Z,13Z,16Z,19Z))\ | |
| ([H])C([H])═C([H])C([H])([H])C([H])═C([H])C([H])([H])C([H])([H])[H])C([H])([H]) | | |
| OC(═O)C([H])([H])C([H])([H])C([H])([H])C([H])([H])C([H])([H])C([H])([H])C([H])([H]) | | |
| C([H])([H])C([H])([H])C([H])([H])C([H])([H])C([H])([H])C([H])([H])C([H])([H]) | | |
| C([H])([H])[H])[C@]1([H])O[H])N1C([H])═C([H])C(═NC1═O)N | | |
| ([H])[H] | | |
| [H]OC1([H])[C@@]([H])(O[C@]([H])(C([H])([H])OP(═O)(O[H])OP(═O)(O | \CDP- | FooDB |
| [H])OC([H])([H])[C@]([H])(OC(═O)C([H])([H])C([H])([H])C([H])([H])C([H]) | DG(18:0/20:4(8Z,11Z, | |
| ([H])C([H])([H])C([H])([H])C([H])═C([H])C([H])([H])C([H])═C([H])C([H])([H])C | 14Z,17Z))\ | |
| ([H])═C([H])C([H])([H])C([H])═C([H])C([H])([H])C([H])([H])[H])C([H])([H])O | | |
| C(═O)C([H])([H])C([H])([H])C([H])([H])C([H])([H])C([H])([H])C([H])([H])C | | |
| ([H])([H])C([H])([H])C([H])([H])C([H])([H])C([H])([H])C([H])([H])C([H])([H])C | | |
| ([H])([H])C([H])([H])C([H])([H])C([H])([H])[H])[C@]1([H])O[H])N1C([H])═C | | |
| ([H])C(═NC1═O)N([H])[H] | | |
| [H]OC1([H])[C@@]([H])(O[C@]([H])(C([H])([H])OP(═O)(O[H])OP(═O)(O | \CDP- | FooDB |
| [H])OC([H])([H])[C@]([H])(OC(═O)C([H])([H])C([H])([H])C([H])([H])C([H]) | DG(18:0/22:3(10Z, | |
| ([H])C([H])([H])C([H])([H])C([H])([H])C([H])([H])C([H])═C([H])C([H])([H])C([H])═ | 13Z,16Z))\ | |
| C([H])C([H])([H])C([H])═C([H])C([H])([H])C([H])([H])C([H])([H])C([H])([H]) | | |
| C([H])([H])[H])C([H])([H])OC(═O)C([H])([H])C([H])([H])C([H])([H])C([H]) | | |
| ([H])C([H])([H])C([H])([H])C([H])([H])C([H])([H])C([H])([H])C([H])([H]) | | |
| C([H])([H])C([H])([H])C([H])([H])C([H])([H])C([H])([H])C([H])([H])C([H])([H])[H]) | | |
| [C@]1([H])O[H])N1C([H])═C([H])C(═NC1═O)N([H])[H] | | |
| COC1═C(CC(OO)C(C)═C)C(O)═C(C═C1)C(═O)\C═C\C1═CC═C(O)C═C1 | Xanthoangelol E | FooDB |
| COC1═CC═C(C(═O)\C═C\C2═CC═C(O)C═C2)C(O)═C1CC(O)C(C)═C | Xanthoangelol D | FooDB |
| OCC(CO)OC═O | Monoacyl glycerol | FooDB |
| [H]OC(═O)C([H])([H])C([H])([H])C([H])([H])C(\[H])═C(\[H])C([H])([H])C(\[H])═ | 12S-HHT | FooDB |
| C(/[H])\C(\[H])═C(/[H])[C@@]([H])(O[H])C([H])([H])C([H])([H])C([H])([H]) | | |
| C([H])([H])C([H])([H])[H] | | |
| [H]OC([H])(C(═O)N([H])C([H])([H])C([H])([H])C(═O)N([H])C([H])([H])C([H]) | \2,4,7,10,13,16,19- | FooDB |
| ([H])SC(═O)C([H])═C([H])C([H])═C([H])C([H])([H])C([H])═C([H])C([H])([H]) | Docosaheptaenoyl- | |
| C([H])═C([H])C([H])([H])C([H])═C([H])C([H])([H])C([H])═C([H])C([H])([H]) | CoA\ | |
| C([H])═C([H])C([H])([H])C([H])([H])[H])C(C([H])([H])[H])(C([H])([H])[H])C | | |
| ([H])([H])OP(═O)(O[H])OP(═O)(O[H])OC([H])([H])[C@@]1([H])O[C@@]([H]) | | |
| (N2C([H])═NC3═C2N═C([H])N═C3N([H])[H])[C@]([H])(O[H])[C@]1([H]) | | |
| OP(═O)(O[H])O[H] | | |
| [H]OC1([H])C([H])([H])C2(OC2(C([H])([H])C(═O)C(═C([H])C([H])═C([H])C | Fucoxanthin | FooDB |
| (═C([H])C([H])═C([H])C([H])═C(C([H])═C([H])C([H])═C(C([H])═C═C2C(O | | |
| [H])(C([H])([H])[H])C([H])([H])C([H])(OC(═O)C([H])([H])[H])C([H])([H])C2(C | | |
| ([H])([H])[H])C([H])([H])[H])C([H])([H])[H])C([H])([H])[H])C([H])([H])[H])C | | |
| ([H])([H])[H])C(C([H])([H])[H])(C([H])([H])[H])C1([H])[H])C([H])([H])[H] | | |
| [H]OC1═NC(═NC(N([H])[H])═C1N(C([H])═O)C([H])([H])[H])N([H])[H] | \2,6-Diamino-4- | FooDB |
| | hydroxy-5-N- | |
| | methylformamidopyrimidine\ | |
| CC(C)C1CCC(C)C═C1O | cis-p-Menth-2-enol | FooDB |
| [H]OC([H])(C(═O)N([H])C([H])([H])C([H])([H])C(═O)N([H])C([H])([H])C([H]) | \Cyclohex-1,5- | FooDB |
| ([H])SC(═O)C1═C([H])C([H])([H])C([H])([H])C([H])═C1[H])C(C([H])([H])[H]) | diene-1-carboxyl- | |
| (C([H])([H])[H])C([H])([H])OP(═O)(O[H])OP(═O)(O[H])OC([H])([H])[C@ | CoA\ | |
| @]1([H])O[C@@]([H])(N2C([H])═NC3═C2N═C([H])N═C3N([H])[H])[C@] | | |
| ([H])(O[H])[C@]1([H])OP(═O)(O[H])O[H] | | |
| [H]OC([H])([H])[C@@]1([H])O[C@]([H])(OC([H])([H])C(═C([H])C([H])([H]) | cis-Zeatin-O- | FooDB |
| N([H])C2═NC([H])═NC3═C2N([H])C([H])═N3)C([H])([H])[H])[C@]([H])(O | glucoside | |
| [H])[C@@]([H])(O[H])[C@]1([H])O[H] | | |
| CCCCCCCCCCCCCC(═O)SCCNC(═O)CCNC(═O)C(O)C(C)(C)COP(O) | Tetradecanoyl-CoA | HMDB |
| (═O)OP(O)(═O)OC[C@H]1O[C@H]([C@H](O)[C@@H]1OP(O)(O)═O)N1 | | |
| C═NC2═C1N═CN═C2N | | |
| NC1═NC(═O)C2═C(NCC(CN(C═O)C3═CC═C(C═C3)C(═O)N[C@@H](C | 10- | HMDB |
| CC(O)═O)C(O)═O)N2)N1 | Formyltetrahydrofolate | |
| O[C@@H]1[C@@H](COP(O)(═O)OP(O)(O)═O)OC([C@@H]1O)N1C═N | IDP | HMDB |
| C2═C1NC═NC2═O | | |
| NCC(═O)CCC(O)═O | 5-Aminolevulinic | HMDB |
| | acid | |
| OC[C@H]1O[C@H](O[C@H]2O[C@H](COP(O)(O)═O)[C@@H](O)[C@ | Trehalose 6- | HMDB |
| H](O)[C@H]2O)[C@H](O)[C@@H](O)[C@@H]1O | phosphate | |
| CC[C@](O)(C(C)═O)C(O)═O | (S)-2-Aceto-2- | HMDB |
| | hydroxybutanoic | |
| | acid | |
| [H][C@@]1(CC═C2C3═C(CC[C@]12C)[C@@]1(C)CC[C@H](O)[C@@ | Delta 8,14-Sterol | HMDB |
| H](C)[C@]1([H])CC3)[C@H](C)CCC(═C)C(C)C | | |
| CC[C@](O)(C(C)═O)C(O)═O | (S)-2-Aceto-2- | HMDB |
| | hydroxybutanoic | |
| | acid | |
| NC1═NC2═C(N═CN2[C@@H]2O[C@H](COP(O)(═O)OP(O)(═O)O[C@H] | Guanosine | HMDB |
| 3O[C@H](CO)[C@@H](O)[C@H](O)[C@@H]3O)[C@@H](O)[C@H]2O) | diphosphate | |
| C(═O)N1 | mannose | |
| O[C@H]1[C@@H](O)[C@@H](O[C@@H]1COP(O)(O)═O)N1C(═O)NC | Orotidylic acid | HMDB |
| (═O)C═C1C(O)═O | | |
| O[C@@H]1[C@H](O)[C@@H](OP(O)(O)═O)[C@H](OP(O)(O)═O)[C@ | Inositol 1,4,5- | HMDB |
| @H](O)[C@@H]1OP(O)(O)═O | trisphosphate | |
| C[C@H](N)C(O)═O | L-Alanine | HMDB |
| [H][C@@]12CCC([C@H](C)CCCC(C)C)[C@@]1(C)CC[C@@]1([H])C2═ | 3-beta-Hydroxy-4- | HMDB |
| CC[C@]2([H])[C@]1(C)CC[C@H](O)[C@@]2(C)C(O)═O | beta-methyl-5- | |
| | alpha-cholest-7- | |
| | ene-4-alpha- | |
| | carboxylate | |
| NC(CCC1═NC═C(C[C@H](N)C(O)═O)N1)C(O)═O | 2-(3-Carboxy-3- | HMDB |
| | aminopropyl)-L- | |
| | histidine | |
| NC(N)═NCCCC(O)═O | 4- | HMDB |
| | Guanidinobutanoic | |
| | acid | |
| OC(═O)[C@@H]1CC(═O)NC(═O)N1 | L-Dihydroorotic | HMDB |
| | acid | |
| CC1═CC2═C(C═C1C)N═CN2 | Dimethylbenzimidazole | HMDB |
| OC(═O)CCCCCCC(═O)NCC(O)═O | Suberylglycine | HMDB |
| N[C@@H](CC1═CC═C(O)C═C1)C(═O)N1CCC[C@H]1C(═O)N[C@@H] | Endomorphin-1 | HMDB |
| (CC1═CNC2═CC═CC═C12)C(═O)N[C@@H](CC1═CC═CC═C1)C(N)═O | | |
| OC1C2(Cl)C3(Cl)C4(Cl)C1(Cl)C1(Cl)C2(Cl)C3(Cl)C(Cl)(Cl)C41Cl | Chlordecone | HMDB |
| | alcohol | |
| OC(═O)CCCNCCC(O)═O | N-Carboxyethyl-g- | HMDB |
| | aminobutyric acid | |
| [H][C@@]12CC[C@@](O)(C(═O)CO)[C@@]1(C)C[C@H](O)[C@@]1 | Cortisol | HMDB |
| ([H])[C@@]2([H])CCC2═CC(═O)CC[C@]12C | | |
| OC[C@H]1O[C@@H](O[C@@H]2CO[C@@H](O)[C@H](O)[C@H]2O) | Galactose-beta- | HMDB |
| [C@H](O)[C@@H](O)[C@H]1O | 1,4-xylose | |
| CCCCC(C)C(═O)SCCNC(═O)CCNC(═O)C(O)C(C)(C)COP(O)(═O)OP(O) | 2-Methylhexanoyl- | HMDB |
| (═O)OC[C@H]1O[C@H]([C@H](O)[C@@H]1OP(O)(O)═O)N1C═NC2═ | CoA | |
| C1N═CN═C2N | | |
| O[C@H]1[C@H](OP(O)(O)═O)[C@@H](OP(O)(O)═O)[C@@H](O)[C@ | 1D-Myo-inositol | HMDB |
| @H](OP(O)(O)═O)[C@@H]1OP(O)(O)═O | 1,3,4,6- | |
| | tetrakisphosphate | |
| O[C@@H]1C═C[C@@H](NCC2═CNC3═C2C(═O)NC(═N)N3)[C@@H]1O | Queuine | HMDB |
| O[C@@H]1COC(═O)[C@H](O)[C@H]1O | D-Xylono-1,5- | HMDB |
| | lactone | |
| CCCCCCCC\C═C/CC(═O)SCCNC(═O)CCNC(═O)[C@H](O)C(C)(C)CO | 3Z-dodecenoyl- | HMDB |
| P(O)(═O)OP(O)(═O)OC[C@H]1O[C@H](C(O)[C@H]1OP(O)(O)═O)N1C═ | CoA | |
| NC2═C(N)N═CN═C12 | | |
| CC(═O)C(═O)[C@H](N)[C@@H](O)[C@@H](OS(═O)(═O)O[C@@H]1[C | Uridine | HMDB |
| @@H](COP(O)(═O)OP(O)(O)═O)O[C@H]([C@@H]1O)N1C═CC(═O)NC | diphosphate | |
| 1═O)[C@H](O)CO | acetylgalactosamine | |
| | 4-sulfate | |
| OC1═NC2N═CN═CC2N1 | 8-Hydroxypurine | HMDB |
| CC(N)C(═O)NCC(O)═O | Alanyl-Glycine | HMDB |
| OC(═O)C(\O)═C\C1═CC═C(O)C═C1 | 2-Hydroxy-3-(4- | HMDB |
| | hydroxyphenyl)propenoic | |
| | acid | |
| COC1═C(O)C═C2[C@H](CC3═CC(O)═C(O)C═C3)N(C)CCC2═C1 | (S)-3-Hydroxy-N- | HMDB |
| | methylcoclaurine | |
| C[C@@H](CCC═C(C)C)C1CCC2C3═C(CC[C@]12C)[C@@]1(C)CC[C | 4a- | HMDB |
| @H](O)[C@](C)(C1CC3)C(O)═O | Methylzymosterol- | |
| | 4-carboxylic acid | |
| N[C@@H](CCCC(═O)OP(O)(═O)OCC1OC([C@H](O)[C@@H]1O)N1C═ | L-2-Aminoadipate | HMDB |
| NC2═C(N)N═CN═C12)C(O)═O | adenylate | |
| [H][C@@](COC(═O)CCCCCCCCCCCCCCC)(COP(O)(═O)OP(O)(═O)O | CDP- | HMDB |
| C[C@H]1O[C@H](C(O)[C@H]1O)N1C═CC(N)═NC1═O)OC(═O)CCCCC | DG(16:0/18:2(9Z,12Z)) | |
| CC\C═C/C\C═C/CCCCC | | |
| [H][C@@](COC(═O)CCCCCCCCCCCCCCCCC)(COP(O)(═O)OP(O)(═O) | CDP- | HMDB |
| OC[C@H]1O[C@H](C(O)[C@H]1O)N1C═CC(N)═NC1═O)OC(═O)CCC | DG(18:0/18:2(9Z,12Z)) | |
| CCCC\C═C/C\C═C/CCCCC | | |
| CC(C)CCCC(C)C1CCC2C3[C@@H](O)CC4CC(═O)CC[C@]4(C)C3CC | 7a-Hydroxy-5b- | HMDB |
| [C@]12C | cholestan-3-one | |
| [H][C@@](COC(═O)CCCCCCCCCCCCCCC)(COP(O)(═O)OP(O)(═O)OC | CDP-DG(16:0/16:0) | HMDB |
| [C@H]1O[C@H](C(O)[C@H]1O)N1C═CC(N)═NC1═O)OC(═O)CCCCC | | |
| CCCCCCCCCC | | |
| [H][C@@](COC(═O)CCCCCCCCCCCCCCCCC)(COP(O)(═O)OP(O)(═O) | CDP-DG(18:0/16:0) | HMDB |
| OC[C@H]1O[C@H](C(O)[C@H]1O)N1C═CC(N)═NC1═O)OC(═O)CCC | | |
| CCCCCCCCCCCC | | |
| OC(C═O)C(O)═O | Tartro-te | HMDB |
| | semialdehyde | |
| CC1═NC═C(C═O)C(C(O)═O)═C1O | 2-Methyl-3- | HMDB |
| | hydroxy-5- | |
| | formylpyridine-4- | |
| | carboxylate | |
| CC1═NC═C(C(O)═O)C(C(O)═O)═C1O | 3-Hydroxy-2- | HMDB |
| | methylpyridine-4,5- | |
| | dicarboxylate | |
| [H][C@@](COC(═O)CCCCCCCCCCCCCCC)(COP(O)(═O)OP(O)(═O)O | CDP- | HMDB |
| C[C@H]1O[C@H](C(O)[C@H]1O)N1C═CC(N)═NC1═O)OC(═O)CCC\C═ | DG(16:0/20:4(5Z,8Z, | |
| C/C\C═C/C\C═C/C\C═C/CCCCC | 11Z,14Z)) | |
| [H][C@@](COC(═O)CCCCCCCCCCCCCCC)(COP(O)(═O)OP(O)(═O)O | CDP- | HMDB |
| C[C@H]1O[C@H](C(O)[C@H]1O)N1C═CC(N)═NC1═O)OC(═O)CCCCC | DG(16:0/20:4(8Z,11Z, | |
| C\C═C/C\C═C/C\C═C/C\C═C/CC | 14Z,17Z)) | |
| [H][C@@](COC(═O)CCCCCCCCCCCCCCC)(COP(O)(═O)OP(O)(═O)O | CDP- | HMDB |
| C[C@H]1O[C@H](C(O)[C@H]1O)N1C═CC(N)═NC1═O)OC(═O)CCCCC | DG(16:0/22:3(10Z, | |
| CCC\C═C/C\C═C/C\C═C/CCCCC | 13Z,16Z)) | |
| [H][C@@](COC(═O)CCCCCCCCCCCCCCC)(COP(O)(═O)OP(O)(═O)O | CDP- | HMDB |
| C[C@H]1O[C@H](C(O)[C@H]1O)N1C═CC(N)═NC1═O)OC(═O)CC\C═C | DG(16:0/22:6(4Z,7Z, | |
| /C\C═C/C\C═C/C\C═C/C\C═C/C\C═C/CC | 10Z,13Z,16Z,19Z)) | |
| [H][C@@](COC(═O)CCCCCCCCCCCCCCCCC)(COP(O)(═O)OP(O)(═O) | CDP- | HMDB |
| OC[C@H]1O[C@H](C(O)[C@H]1O)N1C═CC(N)═NC1═O)OC(═O)CCC\ | DG(18:0/20:4(5Z,8Z, | |
| C═C/C\C═C/C\C═C/C\C═C/CCCCC | 11Z,14Z)) | |
| [H][C@@](COC(═O)CCCCCCCCCCCCCCCCC)(COP(O)(═O)OP(O)(═O) | CDP- | HMDB |
| OC[C@H]1O[C@H](C(O)[C@H]1O)N1C═CC(N)═NC1═O)OC(═O)CCC | DG(18:0/20:4(8Z,11Z, | |
| CCC\C═C/C\C═C/C\C═C/C\C═C/CC | 14Z,17Z)) | |
| [H][C@@](COC(═O)CCCCCCCCCCCCCCCCC)(COP(O)(═O)OP(O)(═O) | CDP- | HMDB |
| OC[C@H]1O[C@H](C(O)[C@H]1O)N1C═CC(N)═NC1═O)OC(═O)CCC | DG(18:0/22:3(10Z, | |
| CCCCC\C═C/C\C═C/C\C═C/CCCCC | 13Z,16Z)) | |
| OC(═O)C(\O)═C\C1═CC═C(O)C═C1 | 2-Hydroxy-3-(4- | HMDB |
| | hydroxyphenyl)propenoic | |
| | acid | |
| COC1═C(O)C═C2[C@H](CC3═CC(O)═C(O)C═C3)N(C)CCC2═C1 | (S)-3-Hydroxy-N- | HMDB |
| | methylcoclaurine | |
| C[C@@H](CCC═C(C)C)C1CCC2C3═C(CC[C@]12C)[C@@]1(C)CC[C | 4a- | HMDB |
| @H](O)[C@](C)(C1CC3)C(O)═O | Methylzymosterol- | |
| | 4-carboxylic acid | |
| [H][C@@](COC(═O)CCCCCCCCCCCCCCC)(COP(O)(═O)OP(O)(═O)O | CDP-DG(16:0/18:0) | HMDB |
| C[C@H]1O[C@H](C(O)[C@H]1O)N1C═CC(N)═NC1═O)OC(═O)CCCCC | | |
| CCCCCCCCCCCC | | |
| [H][C@@](COC(═O)CCCCCCCCCCCCCCC)(COP(O)(═O)OP(O)(═O)O | CDP- | HMDB |
| C[C@H]1O[C@H]([C@@H](O)C1O)N1C═CC(N)═NC1═O)OC(═O)CCC | DG(16:0/18:1(11Z)) | |
| CCCCCC\C═C/CCCCCC | | |
| [H][C@@](COC(═O)CCCCCCCCCCCCCCC)(COP(O)(═O)OP(O)(═O)O | CDP- | HMDB |
| C[C@H]1O[C@H](C(O)[C@H]1O)N1C═CC(N)═NC1═O)OC(═O)CCC | DG(16:0/18:1(9Z)) | |
| CCCC\C═C/CCCCCCCC | | |
| [H][C@@](COC(═O)CCCCCCCCCCCCCCCCC)(COP(O)(═O)OP(O)(═O)O | CDP-DG(18:0/18:0) | HMDB |
| C[C@H]1O[C@H](C(O)[C@H]1O)N1C═CC(N)═NC1═O)OC(═O)CCC | | |
| CCCCCCCCCCCCCC | | |
| [H][C@@](COC(═O)CCCCCCCCCCCCCCCCC)(COP(O)(═O)OP(O)(═O)O | CDP- | HMDB |
| C[C@H]1O[C@H](C(O)[C@H]1O)N1C═CC(N)═NC1═O)OC(═O)CCC | DG(18:0/18:1(11Z)) | |
| CCCCCC\C═C/CCCCCC | | |
| [H][C@@](COC(═O)CCCCCCCCCCCCCCCCC)(COP(O)(═O)OP(O)(═O) | CDP- | HMDB |
| OC[C@H]1O[C@H](C(O)[C@H]1O)N1C═CC(N)═NC1═O)OC(═O)CCC | DG(18:0/18:1(9Z)) | |
| CCCC\C═C/CCCCCCCC | | |
| CCCCC\C═C/C═C/C(CCCCCCCC(O)═O)OO | 9(S)-HPODE | HMDB |
| [H][C@@](COC(═O)CCCCCCCCCCCCCCC)(COP(O)(═O)OP(O)(═O)O | CDP-DG(16:0/18:0) | HMDB |
| C[C@H]1O[C@H](C(O)[C@H]1O)N1C═CC(N)═NC1═O)OC(═O)CCCCC | | |
| CCCCCCCCCCCC | | |
| [H][C@@](COC(═O)CCCCCCCCCCCCCCCCC)(COP(O)(═O)OP(O)(═O) | CDP-DG(18:0/18:0) | HMDB |
| OC[C@H]1O[C@H](C(O)[C@H]1O)N1C═CC(N)═NC1═O)OC(═O)CCC | | |
| CCCCCCCCCCCCCC | | |
| [H][C@@](COC(═O)CCCCCCCCCCCCCCCCC)(COP(O)(═O)OP(O)(═O) | CDP- | HMDB |
| OC[C@H]1O[C@H](C(O)[C@H]1O)N1C═CC(N)═NC1═O)OC(═O)CC\C═ | DG(18:0/22:6(4Z,7Z, | |
| C/C\C═C/C\C═C/C\C═C/C\C═C/C\C═C/CC | 10Z,13Z,16Z,19Z)) | |
| [H][C@@](COC(═O)CCCCCCCCC\C═C/CCCCCC)(COP(O)(═O)OP(O) | CDP- | HMDB |
| (═O)OC[C@H]1O[C@H](C(O)[C@H]1O)N1C═CC(N)═NC1═O)OC(═O)C | DG(18:1(11Z)/16:0) | |
| CCCCCCCCCCCCCC | | |
| [H][C@@]12C[C@H](O)CC[C@]1(C)C1CC[C@]3(C)C(CCC3C1[C@H] | Chenodeoxyglycocholoyl- | HMDB |
| (O)C2)C(C)CCC(═O)SCCNC(═O)CCNC(═O)C(O)C(C)(C)COP(O)(═O)OP | CoA | |
| (O)(═O)OC[C@H]1O[C@H]([C@H](O)[C@@H]1OP(O)(O)═O)N1C═NC2 | | |
| ═C1N═CN═C2N | | |
| CC(CCC(═O)NCC(O)═O)C1CCC2C3[C@H](O)CC4C[C@H](O)CC[C@] | Chenodeoxyglycocholic | HMDB |
| 4(C)C3CC[C@]12C | acid | |
| OC(C═O)C(O)═O | Tartro-te | HMDB |
| | semialdehyde | |
| [H][C@@](COC(═O)CCCCCCCCCCCCCCC)(COP(O)(═O)OP(O)(═O)O | CDP- | HMDB |
| C[C@H]1O[C@H]([C@@H](O)C1O)N1C═CC(N)═NC1═O)OC(═O)CCC | DG(16:0/18:1(11Z)) | |
| CCCCCC\C═C/CCCCCC | | |
| [H][C@@](COC(═O)CCCCCCCCCCCCCCC)(COP(O)(═O)OP(O)(═O)O | CDP- | HMDB |
| C[C@H]1O[C@H](C(O)[C@H]1O)N1C═CC(N)═NC1═O)OC(═O)CCCCC | DG(16:0/18:1(9Z)) | |
| CC\C═C/CCCCCCCC | | |
| [H][C@@](COC(═O)CCCCCCCCCCCCCCCCC)(COP(O)(═O)OP(O)(═O) | CDP- | HMDB |
| OC[C@H]1O[C@H](C(O)[C@H]1O)N1C═CC(N)═NC1═O)OC(═O)CCC | DG(18:0/18:1(11Z)) | |
| CCCCCC\C═C/CCCCCC | | |
| [H][C@@](COC(═O)CCCCCCCCCCCCCCCCC)(COP(O)(═O)OP(O)(═O) | CDP- | HMDB |
| OC[C@H]1O[C@H](C(O)[C@H]1O)N1C═CC(N)═NC1═O)OC(═O)CCC | DG(18:0/18:1(9Z)) | |
| CCCC\C═C/CCCCCCCC | | |
| [H][C@@](COC(═O)CCCCCCCCC\C═C/CCCCCC)(COP(O)(═O)OP(O) | CDP- | HMDB |
| (═O)OC[C@H]1O[C@H](C(O)[C@H]1O)N1C═CC(N)═NC1═O)OC(═O)C | DG(18:1(11Z)/18:0) | |
| CCCCCCCCCCCCCCCC | | |
| [H][C@@](COC(═O)CCCCCCCCC\C═C/CCCCCC)(COP(O)(═O)OP(O) | CDP- | HMDB |
| (═O)OC[C@H]1O[C@H](C(O)[C@H]1O)N1C═CC(N)═NC1═O)OC(═O)C | DG(18:1(11Z)/18:1 | |
| CCCCCCCC\C═C/CCCCCC | (11Z)) | |
| CC1═NC═C(C═O)C(C(O)═O)═C1O | 2-Methyl-3- | HMDB |
| | hydroxy-5- | |
| | formylpyridine-4- | |
| | carboxylate | |
| C[C@@H]1OC(OCC(O)[C@@H](O)[C@H](O)[C@@H](NC(C)═O)C═O) | N-Acetyl-6-O-L- | HMDB |
| [C@@H](O)[C@H](O)[C@@H]1O | fucosyl-D- | |
| | glucosamine | |
| [H][C@@]12CC[C@@H](O)[C@@]1(C)CC[C@@]1([H])[C@@]2([H])C | 5a-Androstane- | HMDB |
| C[C@@]2([H])C[C@H](O)CC[C@]12C | 3a,17a-diol | |
| OC(═O)C\C═C\CC(O)═O | 3-Hexenedioic acid | HMDB |
| CCCCCCCCCCCCCCCCCCCCCCCCC(═O)N[C@@H](CO[C@@H]1O | Glucosylceramide | HMDB |
| [C@H](CO)[C@@H](O)[C@H](O)[C@H]1O)[C@H](O)\C═C\CCCCCCCC | (d18:1/25:0) | |
| CCCCC | | |
| CCCCCCCCCCCCC(O)C(O)═O | 2-Hydroxymyristic | HMDB |
| | acid | |
| NC1═NC2═C(N(C═N)C(CNC3═CC═C(C═C3)C(═O)NC(CCC(O)═O)C(O)═ | 5- | HMDB |
| O)CN2)C(═O)N1 | Formiminotetrahydrofolic | |
| | acid | |
| NC(N)═NCCCC(═O)C(O)═O | 2-Oxoarginine | HMDB |
| CN(CC(O)═O)C(═N)NP(O)(O)═O | Phosphocreatine | HMDB |
| OC[C@H]1O[C@@H](O)[C@H](NS(O)(═O)═O)[C@@H](O)[C@@H]1O | N-Sulfo-D- | HMDB |
| | glucosamine | |
| C[C@@H](N)C(═O)N[C@H](C)C(O)═O | D-Alanyl-D-alanine | HMDB |
| CCCCCCCCC[C@H](O)CC(═O)SCCNC(═O)CCNC(═O)C(O)C(C)(C)CO | (S)-3- | HMDB |
| P(O)(═O)OP(O)(═O)OC[C@H]1O[C@H]([C@H](O)[C@@H]1OP(O)(O)═ | Hydroxydodecanoyl- | |
| O)N1C═NC2═C(N)N═CN═C12 | CoA | |
| OC(═O)C(═O)CC1═CC(O)═CC═C1 | 3- | HMDB |
| | Hydroxyphenylpyruvic | |
| | acid | |
| CC\C═C\CC(═O)SCCNC(═O)CCNC(═O)C(O)C(C)(C)COP(O)(═O)OP(O) | trans-3-Hexenoyl- | HMDB |
| (═O)OC[C@H]1O[C@H]([C@H](O)[C@@H]1OP(O)(O)═O)N1C═NC2═C | CoA | |
| (N)N═CN═C12 | | |
| CC(C)(COP(O)(═O)OP(O)(═O)OC[C@H]1O[C@H]([C@H](O)[C@@H]1 | Phenylacetyl-CoA | HMDB |
| OP(O)(O)═O)N1C═NC2═C(N)N═CN═C12)[C@@H](O)C(═O)NCCC(═O) | | |
| NCCSC(═O)CC1═CC═CC═C1 | | |
| CN1C(N)═NC2═C(NC═N2)C1═O | 1-Methylguanine | HMDB |
| OC[C@H]1OC([C@@H](O)[C@@H]1O)N1C═NC2═C1NC═NC2═O | Arabinosylhypoxanthine | HMDB |
| C═C1C═NC2═CC═CC═C12 | 3-Methylene- | HMDB |
| | indolenine | |
| C\C(\C═C\C═C(/C)\C═C\C1C(C)═CC(═O)CC1(C)C)═C/C═C/C═C(\C)/C═ | E,e-Carotene-3,3′- | HMDB |
| C/C═C(\C)/C═C/C1C(C)═CC(═O)CC1(C)C | dione | |
| CC1═C2CC3═C(CCC(O)═O)C(C)═C(CC4═C(CCC(O)═O)C(C)═C(CC5═ | Coproporphyrinogen I | HMDB |
| C(CCC(O)═O)C(C)═C(CC(N2)═C1CCC(O)═O)N5)N4)N3 | | |
| CCCCC[C@H](O)\C═C\[C@H]1OC(O)C[C@H](O)[C@@H]1C\C═C/CC | Thromboxane B2 | HMDB |
| CC(O)═O | | |
| OC[C@H]1O[C@H](O[C@H]2[C@H](O)[C@@H](O)[C@@H](O[C@H]3 | Maltopentaose | HMDB |
| [C@H](O)[C@@H](O)[C@@H](O[C@H]4[C@H](O)[C@@H](O)[C@@H] | | |
| (O[C@H]5[C@H](O)[C@@H](O)[C@@H](O)O[C@@H]5CO)O[C@@H] | | |
| 4CO)O[C@@H]3CO)O[C@@H]2CO)[C@H](O)[C@@H](O)[C@@H]1O | | |
| CCCCCCCCCCCCCCCCCCCCCCCCC(O)C(═O)NC(COP(O)(═O)OC1 | Mannosyl- | HMDB |
| C(O)C(O)C(OC2OC(CO)C(OP(O)(═O)OC3C(O)C(O)C(O)C(O)C3O)C(O) | diinositol- | |
| C2O)C(O)C1O)C(O)C(O)CCCCCCCCCCCCCC | phosphorylceramide | |
| CC(CCOP(O)(═O)OP(O)(═O)O[C@@H]1O[C@H](CO)[C@@H](O[C@ | Mannosyl-(N- | HMDB |
| @H]2O[C@H](CO)[C@@H](O[C@@H]3O[C@H](CO)[C@@H](O)[C@ | acetylglucosaminyl) | |
| H](O)[C@@H]3O)[C@H](O)[C@H]2NC(C)═O)[C@H](O)[C@H]1NC(C)═ | 2- | |
| O)CC\C═C(/C)CC\C═C(/C)CC\C═C(/C)CC\C═C(/C)CC\C═C(/C)CC\C═C | diphosphodolichol | |
| (/C)CC\C═C(/C)CC\C═C(/C)CC\C═C(/C)CC\C═C(/C)CC\C═C(/C)CC\C═C | | |
| (/C)CC\C═C(/C)CC\C═C(/C)CCC═C(C)C | | |
| C\C(\C═C\C═C(/C)\C═C\C1═C(C)CCCC1(C)C)═C/C═O | Reti-I | HMDB |
| C[C@H](N[C@@H](CCC1═CC═CC═C1)C(O)═O)C(═O)N1[C@H]2CCC | Ramiprilat | HMDB |
| [C@H]2C[C@H]1C(O)═O | | |
| CC1═C(CC(O)═O)C2═C(C═CC(F)═C2)C1═CC1═CC═C(C═C1)S(C)(═O)═ | Sulindac sulfone | HMDB |
| O | | |
| CC1(C)CCSC2═C1C═C(C═C2)C#CC1═NC═C(C═C1)C(O)═O | Tazarotenic acid | HMDB |
| CC1═C2N═C(OC3O[C@@H]([C@@H](O)[C@H](O)[C@H]3O)C(O)═O) | 12- | HMDB |
| C3═C(N═CC═C3)N(C3CC3O)C2═NC═C1 | Hydroxynevirapine | |
| | glucuronide | |
| CC1C2CCC(C)(C═C)N2C(═N)NC1═O | Are-ine | HMDB |
| [H][C@]12OC(O)(C(C)CC1OC)C(═O)C(═O)N1CCCCC1C(═O)OC(C(C)C | 31-O- | HMDB |
| (O)CC(═O)C(CC═C)\C═C(C)/CC(C)C[C@@H]2OC)C(\C)═C\C1CC[C@ | Demethyltacrolimus | |
| H](O)C(O)C1 | | |
| COC1═C(C═C(Cl)C═C1)C(O)═NCCC1═CC═C(C═C1)S(═O)(═O)NC(O)═ | 3-cis- | HMDB |
| N[C@@H]1CCC[C@H](O)C1 | Hydroxycyclohexyl | |
| | glyburide | |
| CN1(═O)CCN(CC1)C(═O)OC1N(C(═O)C2═C1N═CC═N2)C1═NC═C(Cl) | nopiclone N-oxide | HMDB |
| C═C1 | | |
| CN1CCN2C(C1)C1═CC═CC═C1CC1═CC(O)═CN═C21 | 8- | HMDB |
| | hydroxymirtazapine | |
| CC(C)C1═C(\C═C\[C@@H]2C[C@@H](O)CC(═O)O2)C(═NC(═N1)N(C) | Rosuvastatin 5 S- | HMDB |
| S(C)(═O)═O)C1═CC═C(F)C═C1 | lactone | |
| NC(CC(O)CNC(N)═N)C(O)═O | L-erythro-4- | HMDB |
| | Hydroxyarginine | |
| [H][C@]1(O)C[C@@]([H])(O[C@]1([H])COP(O)(═O)OP(O)(═O)OP(O)(O)═ | 2-hydroxy-dATP | HMDB |
| O)N1C═NC2═C(N)N═C(O)N═C12 | | |
| CCC1(C)OC(═O)N═C1O | 5-ethyl-5-methyl- | HMDB |
| | 2,4- | |
| | oxazolidinedione | |
| CSCC[C@@H](N)C(O)═O | D-Methionine | YMDB |
| CCCCC\C═C/C[C@@H](O)\C═C\C═C/C═C/[C@@H](O)CCCC(O)═O | Delta(6)- | YMDB |
| | trans, Delta(8)-cis- | |
| | leukotriene B4 | |
| [H][C@@]1(OC(═O)[C@@H](O)[C@@H]1O)[C@@H](O)CO | L-galactono-1,4- | YMDB |
| | lactone | |
| CCCC(C(C)C)C(═O)OC1O[C@@H]([C@@H](O)[C@H](O)[C@H]1O)C | Valproic acid | BMD |
| (O)═O | glucuronide | |
| [H]C(═O)C(O)C(O)C(OC1OC(CO)C(O)C(OC2OC(CO)C(OC3OC(CO)C | Difucosyllacto-N- | BMD |
| (O)C(OC4OC(CO)C(OC5OC(C)C(O)C(O)C5O)C(OC5OC(CO)C(O)C(O) | hexaose I | |
| C5O)C4NC(C)═O)C3O)C(OC3OC(C)C(O)C(O)C3O)C2NC(C)═O)C1O)C | | |
| (O)CO | | |
| [H][C@]1(O)O[C@@]([H])(CO)[C@](O)(O[C@@]2([H])O[C@@]([H])(CO) | 1,4-beta-D-Glucan | BMD |
| [C@](O)(O[C@@]3([H])O[C@@]([H])(CO)[C@]([H])(O)[C@@]([H])(O) | | |
| [C@]3([H])O)[C@@]([H])(O)[C@]2([H])O)[C@@]([H])(O)[C@]1([H])O | | |
| [H]C(═O)OCC(COP(O)(═O)OC1[C@@H](O)C(O)C(O)[C@H](O)[C@H]1 | 1-Phosphatidyl-D- | BMD |
| O)OC([H])═O | myo-inositol | |
| CCCCCC(O)\C═C\C═C/CCCCCCCC(O)═O | 13(S)-HODE | BMD |
| [H]C(═O)OCC(COP(O)(═O)OC1[C@@H](O)C(O)C(O)[C@H](O)[C@H]1 | 1-Phosphatidyl-D- | BMD |
| O)OC([H])═O | myo-inositol | |
| [H]\C(\C═C\C(\C)═C\C═C\C═C(/C)\C═C\C═C(/C)\C═C\C1═C(C)C[C@@ | (3R,3′R,6′R,9′-cis)- | BMD |
| H](O)CC1(C)C)═C(/C)\C═C\[C@@H]1C(C)═C[C@H](O)CC1(C)C | b,e-Carotene-3,3′- | |
| | diol | |
| OCC1OC(C(O)C(O)C1O)N1C═NC2═C1N═CN═C2NCC1═CC═CO1 | kinetin-9-N- | PMN |
| | glucoside | |
| CCCCCCCCC═CCCCCCCCC(═O)OCC(COP([O−]) | 1-18:1-2-18:1-sn- | PMN |
| (═O)OCC[N+](C)(C)C)OC(═O)CCCCCCCC═CCCCCCCCC | glycerol-3- | |
| | phosphocholine | |
| [N+]C(CO)CC1═CNC═N1 | histidinol | PMN |
| CC(C)═CCCC(C)═CC═O | neral | PMN |
| COC1═C(O)C═CC(C═CC(═O)NCCC2═CC═C(O)C═C2)═C1 | N-feruloyltyramine | PMN |
| CCC═CCC1C(CCCCCCCC([O−])═O)C═CC1═O | \12-oxo-cis-10,15- | PMN |
| | phytodienoate\ | |
| [O−]C(═O)CCC1═C2CC3═C(CCC([O−])═O)C(CC([O−])═O)═ | uroporphyrinogen- | PMN |
| C(CC4═C(CC([O−])═O)C(CCC([O−])═O)═C(CC5═C(CC([O−])═ | III | |
| O)C(CCC([O−])═O)═C(CC(N2)═C1CC([O−])═O)N5)N4)N3 | | |
| OC1CC(O)(CC(═O)C1O)C([O−])═O | 3-dehydroqui-te | PMN |
| CC(C)C(C)C(O)C(O)C(C)C1CCC2C3CC(O)C4CC(O)C(O)CC4(C)C3CC | 6alpha-hydroxy- | PMN |
| C12C | castasterone | |
| CC(═O)C═CC1═C(C)CCCC1(C)C | beta-ionone | PMN |
| CCCCCCCCCCCCCCCC(═O)OC(COC1OC(CO)C(O)C(O)C1O)COC(═ | 1-18:2-2-16:0- | PMN |
| O)CCCCCCCC═CCC═CCCCCC | monogalactosyldiacyl | |
| | glycerol | |
| CC(C)(COP([O−])(═O)OP([O−])(═O)OCC1OC(C(O)C1OP([O−]) | 4-coumaryl-CoA | PMN |
| ([O−])═O)N1C═NC2═C1N═CN═C2N)C(O)C(═O)NCCC(═O)NCCSC(═O)C═C | | |
| C1═CC═C(O)C═C1 | | |
| CC(C)C(═C)CCC(C)C1CC═C2C3═C(CCC12C)C1(C)CCC(O)C(C)C1CC3 | \4alpha-methyl- | PMN |
| | 5alpha-ergosta- | |
| | 8,14,24(28)-trien- | |
| | 3beta-ol\ | |
| CC(═O)SCCNC(═O)CCNC(═O)C(O)C(C)(C)COP([O−])(═O)OP([O−]) | acetyl-CoA | PMN |
| (═O)OCC1OC(C(O)C1OP([O−])([O−])═O)N1C═NC2═C1N═CN═C2N | | |
| CC1(CC2C34OC3CC3C5(C)CCC(OC6OCC(OC7OC(CO)C(O)C(O)C7O) | ave-cin A-2 | PMN |
| C(O)C6OC6OC(CO)C(O)C(O)C6O)C(C)(CO)C5CCC3(C)C4(C)CC(O)C | | |
| 2(C)CC1OC(═O)C1═CC═CC═C1)C═O | | |
| COC1═CC(C═CC(═O)OC(C(O)C(O)C(O)C([O−])═O)C([O−])═ | O-si-poylglucarate | PMN |
| O)═CC(OC)═C1O | | |
| OC1C(O)C(OC1COP([O−])([O−])═O)[N+]1═CC(═CC═C1)C([O−])═O | beta-nicoti-te D- | PMN |
| | ribonucleotide | |
| CCC═CCC═CC═CC(CCCCCCCC([O−])═O)OO | 9(S)-HPOTE | PMN |
| COC1═CC(C═CC(═O)SCCNC(═O)CCNC(═O)C(O)C(C)(C)COP([O−]) | si-poyl-CoA | PMN |
| (═O)OP([O−])(═O)OCC2OC(C(O)C2OP([O−])([O−])═ | | |
| O)N2C═NC3═C2N═CN═C3N)═CC(OC)═C1O | | |
| OCC1OC(OC2═C(C═CC([O−])═O)C═CC═C2)C(O)C(O)C1O | trans-beta-D- | PMN |
| | glucosyl-2- | |
| | hydroxycin-mate | |
| OC1C(O)C(O)C(OP([O−])([O−])═O)C(OP([O−])([O−])═O)C1O | \D-myo-inositol | PMN |
| | (1,2)bisphosphate\ | |
| CN1CC2═C3OCOC3═CC═C2C2═C1C1═C(C═C2)C═C2OCOC2═C1 | dihydrosangui-rine | PMN |
| COC1═CC(C═CC(═O)OCC[N+](C)(C)C)═CC(OC)═C1O | O-si-poylcholine | PMN |
| CC12CCCC(C)(C1C(C═O)C13CC(CCC21)C(═C)C3)C([O−])═O | gibberellin A12- | PMN |
| | aldehyde | |
| CC(CCNC1═NC═NC2═C1N═CN2C1OC(CO)C(O)C(O)C1O)COC1OC(C | dihydrozeatin-9-N- | PMN |
| O)C(O)C(O)C1O | glucoside-O- | |
| | glucoside | |
| CSCCCCCCCC(SC1OC(CO)C(O)C(O)C1O)═NO | 7- | PMN |
| | methylthioheptylde | |
| | sulfoglucosinolate | |
| CCCCCCCCCCCCCCCCCCCCCCCCCC(═O)SCCNC(═O)CCNC(═O)C | cerotoyl-CoA | PMN |
| (O)C(C)(C)COP([O−])(═O)OP([O−])(═O)OCC1OC(C(O)C1OP([O−])([O−])═ | | |
| O)N1C═NC2═C1N═CN═C2N | | |
| CC(C)═CC(═O)SCCNC(═O)CCNC(═O)C(O)C(C)(C)COP([O−]) | 3-methylcrotonyl- | PMN |
| (═O)OP([O−])(═O)OCC1OC(C(O)C1OP([O−])([O−])═ | CoA | |
| O)N1C═NC2═C1N═CN═C2N | | |
| COC1═CC(C═CCO)═CC(OC)═C1OC1OC(CO)C(O)C(O)C1O | syringin | PMN |
| NC1═NC2═C(NC3C(N2)OC(COP([O−])(═O)OP([O−]) | molybdopterin | PMN |
| (═O)OCC2OC(C(O)C2O)N2C═NC4═C2N═CN═C4N)C([S−])═ | adenine | |
| C3S)C(═O)N1 | dinucleotide | |
| COC1═CC═C(C═CC)C═C1 | t-anethole | PMN |
| [H][C@@]12CC[C@H]([C@H](C)CCCC(C)C)[C@@]1(C)CC[C@@]1([H]) | CHOLESTERYL | Nature |
| [C@@]2([H])CC═C2CC(Cl)CC[C@]12C | CHLORIDE | |
| CCCC(═O)OC1CC[C@]2(C)C3CC[C@]4(C)[C@H](CCC4C3CC═C2C1) | CHOLESTERYL | Nature |
| [C@H](C)CCCC(C)C | BUTYRATE | |
| CCCCC\C═C\C(═O)OCC1═CN═C(C)C(O)═C1COC(═O)\C═C\CCCCC | PYRIDOXINE | Nature |
| | DIOCTENOATE | |
| CCCCCCCCCCCCCCCC(═O)OCC1═CN═C(C)C(O)═C1COC(═O)CCCC | PYRIDOXINE | Nature |
| CCCCCCCCCCC | DIPALMITATE | |
| CC(═O)OC(C)(C)CCC(═O)C(C)(O)C1C(O)CC2(C)C3CC═C4C(CC(O)C | Cucurbitacin B | Nature |
| (═O)C4(C)C)C3(C)C(═O)CC12C | | |
| Cc1sc2nc(nc(N)c2c1C)-c1ccc(C)cc1 | 5,6-dimethyl-2-(4- | Nature |
| | methylphenyl)thieno | |
| | [2,3-d]pyrimidin-4- | |
| | amine | |
| COc1ccccc1C(═O)N1CCC(N)CC1 | 1-(2- | Nature |
| | methoxybenzoyl)piperidin- | |
| | 4-amine | |
| | hydrochloride | |
| Cc1c(F)cccc1NS(═O)(═O)N1CCCC1 | N-(3-fluoro-2- | Nature |
| | methylphenyl)pyrrolidine- | |
| | 1-sulfo-mide | |
| Cc1sc2nc(COC(═O)c3ccc(O)cc3)nc(N)c2c1C | {4-amino-5,6- | Nature |
| | dimethylthieno[2,3- | |
| | d]pyrimidin-2- | |
| | yl}methyl 4- | |
| | hydroxybenzoate | |
| CNc1ncnc2sc(C)c(C)c12 | N,5,6- | Nature |
| | trimethylthieno[2,3- | |
| | d]pyrimidin-4-amine | |
| CCCCSc1nc(N)c2c(C)c(C)sc2n1 | 2-(butylsulfanyl)- | Nature |
| | 5,6- | |
| | dimethylthieno[2,3- | |
| | d]pyrimidin-4-amine | |
| CC(C)CNC(═O)C(C)(C)C1═CC═C(C═C1)C1═CC(═CN═C1)C#N | 2-(4-(5- | Nature |
| | cyanopyridin-3- | |
| | yl)phenyl)-N- | |
| | isobutyl-2- | |
| | methylpropamide_8 | |
| Cc1sc2nc(CNCc3ccc(Cl)cc3)nc(NCc3ccc(Cl)cc3)c2c1C | N-[(4- | Nature |
| | chlorophenyl)methyl]- | |
| | 2-({[(4- | |
| | chlorophenyl)methyl] | |
| | amino}methyl)- | |
| | 5,6- | |
| | dimethylthieno[2,3- | |
| | d]pyrimidin-4- | |
| | amine; bis(oxalic | |
| | acid) | |
| COCc1nc2sc(C)c(C)c2c(═O)[nH]1 | 2-(methoxymethyl)- | Nature |
| | 5,6-dimethyl- | |
| | 3H,4H-thieno[2,3- | |
| | d]pyrimidin-4-one | |
| Cc1sc2ncnc(N3CCC(═CC3)c3ccc(O)cc3)c2c1C | 4-(1-{5,6- | Nature |
| | dimethylthieno[2,3- | |
| | d]pyrimidin-4-yl}- | |
| | 1,2,3,6- | |
| | tetrahydropyridin-4- | |
| | yl)phenol | |
| CN(C)c1ccc(CNc2nc(C)nc3sc4CCCc4c23)cc1 | N-{[4- | Nature |
| | (dimethylamino)phenyl] | |
| | methyl}- | |
| | 10- | |
| | methyl-7-thia-9,11- | |
| | diazatricyclo[6.4.0.0?, | |
| | ?]dodeca- | |
| | 1(12),2(6),8,10- | |
| | tetraen-12-amine | |
| CN(C)CCNc1ncnc2sc(C(═O)NCc3ccco3)c(C)c12 | 4-{[2- | Nature |
| | (dimethylamino)ethyl] | |
| | amino}-N-[(furan- | |
| | 2-yl)methyl]-5- | |
| | methylthieno[2,3- | |
| | d]pyrimidine-6- | |
| | carboxamide | |
| Cc1cc2c([nH]c(═S)[nH]c2═O)s1 | 6-methyl-2- | Nature |
| | sulfanylidene- | |
| | 1H,2H,3H,4H- | |
| | thieno[2,3- | |
| | d]pyrimidin-4-one | |
| COCCN1C(═O)CCc2cc(O)ccc12 | 6-hydroxy-1-(2- | Nature |
| | methoxyethyl)- | |
| | 1,2,3,4- | |
| | tetrahydroquinolin- | |
| | 2-one | |
| Cc1sc2ncnc(—c3ccc(N)nc3)c2c1C | 5-{5,6- | Nature |
| | dimethylthieno[2,3- | |
| | d]pyrimidin-4- | |
| | yl}pyridin-2-amine | |
| Cc1sc2nc(CN3CCOCC3)nc(NNC(═O)c3ccccc3)c2c1C | N′-{5,6-dimethyl-2- | Nature |
| | [(morpholin-4- | |
| | yl)methyl]thieno[2,3- | |
| | d]pyrimidin-4- | |
| | yl}benzohydrazide | |
| Cc1sc2nc(CNc3cccc(Cl)c3)[nH]c(═O)c2c1C | 2-{[(3- | Nature |
| | chlorophenyl)amino] | |
| | methyl}-5,6- | |
| | dimethyl-3H,4H- | |
| | thieno[2,3- | |
| | d]pyrimidin-4-one | |
| CCn1c(CNc2ncnc3sc(C)c(C)c23)nc2ccccc12 | N-[(1-ethyl-1H-1,3- | Nature |
| | benzodiazol-2- | |
| | yl)methyl]-5,6- | |
| | dimethylthieno[2,3- | |
| | d]pyrimidin-4-amine | |
| Cc1sc2nc(nc(NCc3ccc(Cn4cncn4)cc3)c2c1C)-c1cccnc1 | 5,6-dimethyl-2- | Nature |
| | (pyridin-3-yl)-N-({4- | |
| | [(1H-1,2,4-triazol-1- | |
| | yl)methyl]phenyl}methyl) | |
| | thieno[2,3- | |
| | d]pyrimidin-4-amine | |
| Cc1sc2ncnc(C1)c2c1C | 4-chloro-5,6- | Nature |
| | dimethylthieno[2,3- | |
| | d]pyrimidine | |
| Cc1ccc(Nc2ncnc3sccc23)cc1 | N-(4- | Nature |
| | methylphenyl)thieno | |
| | [2,3-d]pyrimidin-4- | |
| | amine | |
| Cc1sc2nc(Cc3ccccc3)[nH]c(═O)c2c1C | 2-benzyl-5,6- | Nature |
| | dimethyl-3H,4H- | |
| | thieno[2,3- | |
| | d]pyrimidin-4-one | |
| Cc1sc2[nH]c(CN3CCNCC3)nc(═O)c2c1C | 5,6-dimethyl-2- | Nature |
| | [(piperazin-1- | |
| | yl)methyl]-1H,4H- | |
| | thieno[2,3- | |
| | d]pyrimidin-4-one | |
| | dihydrochloride | |
| Cc1sc2nc(nc(N3CCC3)c2c1C)-c1cccnc1 | 3-[4-(azetidin-1-yl)- | Nature |
| | 5,6- | |
| | dimethylthieno[2,3- | |
| | d]pyrimidin-2- | |
| | yl]pyridine | |
| CC1(C)Cc2c(CO1)sc1n(CC═C)c(═O)nc(N)c21 | 3-amino-12,12- | Nature |
| | dimethyl-6-(prop-2- | |
| | en-1-yl)-11-oxa-8- | |
| | thia-4,6- | |
| | diazatricyclo[7.4.0.0?, | |
| | ?]trideca- | |
| | 1(9),2(7),3-trien-5- | |
| | one | |
| CCc1c(C)sc2nc(CC)[nH]c(═O)c12 | 2,5-diethyl-6- | Nature |
| | methyl-3H,4H- | |
| | thieno[2,3- | |
| | d]pyrimidin-4-one | |
| Cc1sc2nc(SC(C(N)═O)c3ccccc3)nc(N)c2c1C | 2-({4-amino-5,6- | Nature |
| | dimethylthieno[2,3- | |
| | d]pyrimidin-2- | |
| | yl}sulfanyl)-2- | |
| | phenylacetamide | |
| Cc1sc2nc(COC(═O)CSc3ccc(F)cc3)nc(N)c2c1C | {4-amino-5,6- | Nature |
| | dimethylthieno[2,3- | |
| | d]pyrimidin-2- | |
| | yl}methyl 2-[(4- | |
| | fluorophenyl)sulfanyl] | |
| | acetate | |
| CC1CCN(Cc2nc(NCc3ccccc3CO)c3c(C)c(C)sc3n2)CC1 | {2-[({5,6-dimethyl- | Nature |
| | 2-[(4- | |
| | methylpiperidin-1- | |
| | yl)methyl]thieno[2,3- | |
| | d]pyrimidin-4- | |
| | yl}amino)methyl]phenyl} | |
| | methanol | |
| C1c1ccc(cc1)C(═O)Nc1ncnc2sc3CCCCc3c12 | 4-chloro-N-{8-thia- | Nature |
| | 4,6- | |
| | diazatricyclo[7.4.0.0?, | |
| | ?]trideca- | |
| | 1(9),2,4,6-tetraen- | |
| | 3-yl}benzamide | |
| COc1ccc(Nc2nc(C)nc3sc4CCCc4c23)cc1OC | N-(3,4- | Nature |
| | dimethoxyphenyl)- | |
| | 10-methyl-7-thia- | |
| | 9,11- | |
| | diazatricyclo[6.4.0.0?, | |
| | ?]dodeca- | |
| | 1(12),2(6),8,10- | |
| | tetraen-12-amine | |
| CCc1cc2c(Nc3ccncc3)ncnc2s1 | N-{6- | Nature |
| | ethylthieno[2,3- | |
| | d]pyrimidin-4- | |
| | yl}pyridin-4-amine | |
| Nc1nc(O)nc2sc3CCCCc3c12 | 3-amino-8-thia-4,6- | Nature |
| | diazatricyclo[7.4.0.0?, | |
| | ?]trideca- | |
| | 1(9),2,4,6-tetraen- | |
| | 5-ol | |
| NNc1nc(N)nc2sc3CCCCc3c12 | 3-hydrazinyl-8-thia- | Nature |
| | 4,6- | |
| | diazatricyclo[7.4.0.0?, | |
| | ?]trideca- | |
| | 1(9),2,4,6-tetraen- | |
| | 5-amine | |
| CCC(C)Cc1c(C)sc2nc(CNc3ccccc3)[nH]c(═O)c12 | 6-methyl-5-(2- | Nature |
| | methylbutyl)-2- | |
| | [(phenylamino)methyl]- | |
| | 3H,4H- | |
| | thieno[2,3- | |
| | d]pyrimidin-4-one | |
| CN(CCC(O)c1ccccc1)c1ncnc2sc(C(O)═O)c(C)c12 | 4-[(3-hydroxy-3- | Nature |
| | phenylpropyl)(methyl) | |
| | amino]-5- | |
| | methylthieno[2,3- | |
| | d]pyrimidine-6- | |
| | carboxylic acid | |
| CN(Cc1ccc(Cl)cc1)c1ncnc2sc3CCCCCc3c12 | N-[(4- | Nature |
| | chlorophenyl)methyl]- | |
| | N-methyl-8-thia- | |
| | 4,6- | |
| | diazatricyclo[7.5.0.0?, | |
| | ?]tetradeca- | |
| | 1(9),2,4,6-tetraen- | |
| | 3-amine | |
| Cc1c(sc2ncnc(NCC3CCCO3)c12)C(═O)NCCC1═CCCCC1 | N-[2-(cyclohex-1- | Nature |
| | en-1-yl)ethyl]-5- | |
| | methyl-4-{[(oxolan- | |
| | 2- | |
| | yl)methyl]amino}thieno | |
| | [2,3- | |
| | d]pyrimidine-6- | |
| | carboxamide | |
| Nc1ncnc2sc3CNCCc3c12 | 8-thia-4,6,11- | Nature |
| | triazatricyclo[7.4.0. | |
| | 0?,?]trideca- | |
| | 1(9),2,4,6-tetraen- | |
| | 3-amine | |
| | hydrochloride | |
| CC1CCN(CC1)c1nc(CN2CCCC2)nc2sc(C)c(C)c12 | 1-{5,6-dimethyl-2- | Nature |
| | [(pyrrolidin-1- | |
| | yl)methyl]thieno[2,3- | |
| | d]pyrimidin-4-yl}-4- | |
| | methylpiperidine | |
| CN1CCN(CC1)c1ncnc2sc(C(═O)NCC═C)c(C)c12 | 5-methyl-4-(4- | Nature |
| | methylpiperazin-1- | |
| | yl)-N-(prop-2-en-1- | |
| | yl)thieno[2,3- | |
| | d]pyrimidine-6- | |
| | carboxamide | |
| COCCCNc1ncnc2sc(C(═O)NCCN3CCCC3)c(C)c12 | 4-[(3- | Nature |
| | methoxypropyl)amino]- | |
| | 5-methyl-N-[2- | |
| | (pyrrolidin-1- | |
| | yl)ethyl]thieno[2,3- | |
| | d]pyrimidine-6- | |
| | carboxamide | |
| CCn1cnnc1CNc1ncnc2sc3CCCCCc3c12 | N-[(4-ethyl-4H- | Nature |
| | 1,2,4-triazol-3- | |
| | yl)methyl]-8-thia- | |
| | 4,6- | |
| | diazatricyclo[7.5.0. | |
| | 0?,?]tetradeca- | |
| | 1(9),2,4,6-tetraen- | |
| | 3-amine | |
| Cc1sc2ncnc(N3CCCC(C3)C(═O)NCC3CC3)c2c1C | N- | Nature |
| | (cyclopropylmethyl)- | |
| | 1-{5,6- | |
| | dimethylthieno[2,3- | |
| | d]pyrimidin-4- | |
| | yl}piperidine-3- | |
| | carboxamide | |
| Cc1sc2[nH]c(CCC(O)═O)nc(═O)c2c1C | 3-{5,6-dimethyl-4- | Nature |
| | oxo-1H,4H- | |
| | thieno[2,3- | |
| | d]pyrimidin-2- | |
| | yl}propanoic acid | |
| [O−][N+](═O)c1cccc(c1)C(═O)Nc1ncnc2sc3CCCc3c12 | 3-nitro-N-{7-thia- | Nature |
| | 9,11- | |
| | diazatricyclo[6.4.0. | |
| | 0?,?]dodeca- | |
| | 1(12),2(6),8,10- | |
| | tetraen-12- | |
| | yl}benzamide | |
| Cc1c(sc2ncnc(NCCCN3CCCC3═O)c12)C(═O)NCCc1ccccc1 | 5-methyl-4-{[3-(2- | Nature |
| | oxopyrrolidin-1- | |
| | yl)propyl]amino}-N- | |
| | (2- | |
| | phenylethyl)thieno | |
| | [2,3-d]pyrimidine-6- | |
| | carboxamide | |
| Cc1cc2c(N)nc(═O)n(CC(═O)NCc3ccccc3)c2s1 | 2-{4-amino-6- | Nature |
| | methyl-2-oxo- | |
| | 1H,2H-thieno[2,3- | |
| | d]pyrimidin-1-yl}-N- | |
| | benzylacetamide | |
| Clc1ccc(cc1)C(═O)Nc1ncnc2sc3CCCc3c12 | 4-chloro-N-{7-thia- | Nature |
| | 9,11- | |
| | diazatricyclo[6.4.0. | |
| | 0?,?]dodeca- | |
| | 1(12),2(6),8,10- | |
| | tetraen-12- | |
| | yl}benzamide | |
| Cc1nnc(NC(═O)C2CCN(CC2)c2ncnc3sc(C)c(C)c23)o1 | 1-{5,6- | Nature |
| | dimethylthieno[2,3- | |
| | d]pyrimidin-4-yl}-N- | |
| | (5-methyl-1,3,4- | |
| | oxadiazol-2- | |
| | yl)piperidine-4- | |
| | carboxamide | |
| C(C1CCN(CC1)c1ncnc2sc3CCCc3c12)c1ccccc1 | 12-(4- | Nature |
| | benzylpiperidin-1- | |
| | yl)-7-thia-9,11- | |
| | diazatricyclo[6.4.0. | |
| | 0?,?]dodeca- | |
| | 1(12),2(6),8,10- | |
| | tetraene | |
| CCn1cnnc1CNc1nc(CN2CCCC2)nc2sc(C)c(C)c12 | N-[(4-ethyl-4H- | Nature |
| | 1,2,4-triazol-3- | |
| | yl)methyl]-5,6- | |
| | dimethyl-2- | |
| | [(pyrrolidin-1- | |
| | yl)methyl]thieno[2,3- | |
| | d]pyrimidin-4- | |
| | amine | |
| CCc1cccc(NC(C)c2nc3sc(C)c(C)c3c(═O)[nH]2)c1 | 2-{1-[(3- | Nature |
| | ethylphenyl)amino] | |
| | ethyl}-5,6-dimethyl- | |
| | 3H,4H-thieno[2,3- | |
| | d]pyrimidin-4-one | |
| C(Nc1ncnc2sc3CCCCc3c12)C1COc2ccccc2C1 | N-[(3,4-dihydro-2H- | Nature |
| | 1-benzopyran-3- | |
| | yl)methyl]-8-thia- | |
| | 4,6- | |
| | diazatricyclo[7.4.0. | |
| | 0?,?]trideca- | |
| | 1(9),2,4,6-tetraen- | |
| | 3-amine | |
| NC(═O)CCOc1ccccc1NS(N)(═O)═O | 3-[2- | Nature |
| | (sulfamoylamino)phenoxy] | |
| | propa-mide | |
| COc1c(C)ccc(NS(N)(═O)═O)c1C | N-(3-methoxy-2,4- | Nature |
| | dimethylphenyl)amino | |
| | sulfo-mide | |
| CCN1[C@H]([C@@H](CC1═O)C(O)═O)c1ccc(OC)cc1OC | (2R,3R)-2-(2,4- | Nature |
| | dimethoxyphenyl)- | |
| | 1-ethyl-5- | |
| | oxopyrrolidine-3- | |
| | carboxylic acid | |
| COc1cc(F)c(F)cc1C(═O)C1CCN(CC1)C(C)═O | 1-[4-(4,5-difluoro-2- | Nature |
| | methoxybenzoyl)piperidin- | |
| | 1-yl]ethan- | |
| | 1-one | |
| NC(═N)c1cc(F)ccc1NS(N)(═O)═O | 5-fluoro-2- | Nature |
| | (sulfamoylamino)benzene- | |
| | 1- | |
| | carboximidamide | |
| Cc1ccc(NCc2cccc3cccnc23)cc1NS(C)(═O)═O | N-(2-methyl-5- | Nature |
| | {[(quinolin-8- | |
| | yl)methyl]amino}phenyl) | |
| | methanesulfo- | |
| | mide | |
| FC(F)(F)c1ccc(CCNc2ncnc3sc4CCCc4c23)cc1 | N-{2-[4- | Nature |
| | (trifluoromethyl)phenyl] | |
| | ethyl}-7-thia- | |
| | 9,11- | |
| | diazatricyclo[6.4.0. | |
| | 0?,?]dodeca- | |
| | 1(12),2(6),8,10- | |
| | tetraen-12-amine | |
| CC(═O)N1CCC(CC1)NCc1ccc2OCCc2c1 | 1-(4-{[(2,3-dihydro- | Nature |
| | 1-benzofuran-5- | |
| | yl)methyl]amino}piperidin- | |
| | 1-yl)ethan-1- | |
| | one | |
| CN(C)CCNc1ncnc2sc(C(═O)N(C)C)c(C)c12 | 4-{[2- | Nature |
| | (dimethylamino)ethyl] | |
| | amino}- | |
| | N,N,5- | |
| | trimethylthieno[2,3- | |
| | d]pyrimidine-6- | |
| | carboxamide | |
| CC(═O)Nc1ccc(N\C(═N/S(═O)(═O)c2ccccc2)c2ccccc2)cc1 | N-{4-[(Z)-N′- | Nature |
| | (benzenesulfonyl)benzene | |
| | imidamido]phenyl} | |
| | acetamide | |
| Cc1ccc(N\C(═N/S(═O)(═O)c2ccc(Cl)cc2)c2ccc(C)cc2)cc1 | (Z)-N′-(4- | Nature |
| | chlorobenzenesulfonyl)- | |
| | 4-methyl-N-(4- | |
| | methylphenyl)benzene- | |
| | 1- | |
| | carboximidamide | |
| NS(═O)(═O)Nc1ccccc1C(═O)NC1CC1 | N-cyclopropyl-2- | Nature |
| | (sulfamoylamino)benzamide | |
| CCC(Cl)C(═O)N1CCc2c1cccc2OC | 2-chloro-1-(4- | Nature |
| | methoxy-2,3- | |
| | dihydro-1H-indol-1- | |
| | yl)butan-1-one | |
| CC(C)CN1C(═O)CCc2cc(O)ccc12 | 6-hydroxy-1-(2- | Nature |
| | methylpropyl)- | |
| | 1,2,3,4- | |
| | tetrahydroquinolin- | |
| | 2-one | |
| COc1cccc2N(CCc12)C(═O)CN═[N+]═[N−] | 2-azido-1-(4- | Nature |
| | ethoxy-2,3- | |
| | dihydro-1H-indol-1- | |
| | yl)ethan-1-one | |
| O═S(═O)(NCc1ccccc1)Nc1ccccc1OCc1ccccn1 | N-benzyl({2- | Nature |
| | [(pyridin-2- | |
| | yl)methoxy]phenyl} | |
| | amino)sulfo-mide | |
| Cc1sc2nc(Cc3ccc(cc3)[N+]([O−])═O)[nH]c(═O)c2c1C | 5,6-dimethyl-2-[(4- | Nature |
| | nitrophenyl)methyl]- | |
| | 3H,4H-thieno[2,3- | |
| | d]pyrimidin-4-one | |
| Cc1ccccc1N\C(═N/S(═O)(═O)c1ccc(Cl)cc1)c1ccccc1 | (Z)-N′-(4- | Nature |
| | chlorobenzenesulfonyl)- | |
| | N-(2- | |
| | methylphenyl)benzene | |
| | carboximidamide | |
| CN(C)S(═O)(═O)Nc1cc(ccc1C)C(F)(F)F | dimethyl({[2- | Nature |
| | methyl-5- | |
| | (trifluoromethyl)phenyl] | |
| | sulfamoyl})amine | |
| O═S(═O)(Nc1nccs1)c1ccc(N\C(═N/S(═O)(═O)c2ccccc2)c2ccccc2)cc1 | (Z)-N′- | Nature |
| | (benzenesulfonyl)- | |
| | N-{4-[(1,3-thiazol-2- | |
| | yl)sulfamoyl]phenyl} | |
| | benzenecarboximidamide | |
| CN(C)Cc1nc2sc(C)c(C)c2c(═O)[nH]1 | 2- | Nature |
| | [(dimethylamino)methyl]- | |
| | 5,6-dimethyl- | |
| | 3H,4H-thieno[2,3- | |
| | d]pyrimidin-4-one | |
| CC(═O)N1CCC(CC1)C(═O)c1ccc2OCCc2c1 | 1-[4-(2,3-dihydro-1- | Nature |
| | benzofuran-5- | |
| | carbonyl)piperidin- | |
| | 1-yl]ethan-1-one | |
| CC1CCCC(C)N1C1═NS(═O)(═O)c2cc(ccc12)[N+]([O−])═O | 3-(2,6- | Nature |
| | dimethylpiperidin-1- | |
| | yl)-6-nitro-1??,2- | |
| | benzothiazole-1,1- | |
| | dione | |
| COc1ccccc1CC(═O)N1CCC2(CC1)NN(C(═O)N2)c1cccc(F)c1 | 2-(3-fluorophenyl)- | Nature |
| | 8-[2-(2- | |
| | methoxyphenyl)acetyl]- | |
| | 1,2,4,8- | |
| | tetraazaspiro[4.5]decan- | |
| | 3-one | |
| CNC(═O)c1ccc(NC(═O)NC2CCOc3ccccc23)c(C)c1 | 4-{[(3,4-dihydro-2H- | Nature |
| | 1-benzopyran-4- | |
| | yl)carbamoyl]amino}- | |
| | N,3- | |
| | dimethylbenzamide | |
| CCOc1cc2CC(C)Oc2cc1NS(N)(═O)═O | N-(5-ethoxy-2- | Nature |
| | methyl-2,3-dihydro- | |
| | 1-benzofuran-6- | |
| | yl)aminosulfo-mide | |
| CC(═O)N1CCC2C(C1)Oc1ccc(N)cc21 | 1-{12-amino-8-oxa- | Nature |
| | 5- | |
| | azatricyclo[7.4.0.0?, | |
| | ?]trideca- | |
| | 1(13),9,11-trien-5- | |
| | yl}ethan-1-one | |
| COc1ccc(Nc2nc(Cc3ccccc3)nc3sc4CCCCc4c23)c(OC)c1 | 5-benzyl-N-(2,4- | Nature |
| | dimethoxyphenyl)- | |
| | 8-thia-4,6- | |
| | diazatricyclo[7.4.0.0?, | |
| | ?]trideca- | |
| | 1(9),2,4,6-tetraen- | |
| | 3-amine | |
| CCCCNC(═O)c1sc2nc(C)nc(N3CCCC3)c2c1C | N-butyl-2,5- | Nature |
| | dimethyl-4- | |
| | (pyrrolidin-1- | |
| | yl)thieno[2,3- | |
| | d]pyrimidine-6- | |
| | carboxamide | |
| COc1ccc(CN2CC(CCC2═O)C(O)═O)c(OC)c1 | 1-[(2,4- | Nature |
| | dimethoxyphenyl)methyl]- | |
| | 6- | |
| | oxopiperidine-3- | |
| | carboxylic acid | |
| CC(C)Oc1ccc(NS(N)(═O)═O)c(C)c1 | N-[2-methyl-4- | Nature |
| | (propan-2- | |
| | yloxy)phenyl]amino | |
| | sulfo-mide | |
| O═Cc1ccccc1OCC(═O)N1CCCCC1 | 2-[2-oxo-2- | Nature |
| | (piperidin-1- | |
| | yl)ethoxy]benzaldehyde | |
| CC(C)(C)C1CCc2c(C1)sc1[nH]c(═S)[nH]c(═S)c21 | 11-tert-butyl-8-thia- | Nature |
| | 4,6- | |
| | diazatricyclo[7.4.0.0?, | |
| | ?]trideca- | |
| | 1(9),2(7)-diene-3,5- | |
| | dithione | |
| COc1ccccc1CN1CCC(═O)CC1═O | 1-[(2- | Nature |
| | methoxyphenyl)methyl] | |
| | piperidine-2,4- | |
| | dione | |
| COC(═O)Cc1nc(N2CCN(CC2)c2ccccc2)c2c(C)c(C)sc2n1 | methyl 2-[5,6- | Nature |
| | dimethyl-4-(4- | |
| | phenylpiperazin-1- | |
| | yl)thieno[2,3- | |
| | d]pyrimidin-2- | |
| | yl]acetate | |
| CCN(CC)Cc1nc(N2CCN(Cc3nc4ccccc4c(═O)[nH]3)CC2)c2c(C)c(C)sc2n1 | 2-[(4-{2- | Nature |
| | [(diethylamino)methyl]- | |
| | 5,6- | |
| | dimethylthieno[2,3- | |
| | d]pyrimidin-4- | |
| | yl}piperazin-1- | |
| | yl)methyl]-3,4- | |
| | dihydroqui-zolin-4- | |
| | one | |
| CCCn1c(SCc2nc(N)c3c(C)c(C)sc3n2)n[nH]c1═O | 3-[({4-amino-5,6- | Nature |
| | dimethylthieno[2,3- | |
| | d]pyrimidin-2- | |
| | yl}methyl)sulfanyl]- | |
| | 4-propyl-4,5- | |
| | dihydro-1H-1,2,4- | |
| | triazol-5-one | |
| Cc1sc2ncnc(NCC3CCCN(C3)S(C)(═O)═O)c2c1C | N-[(1- | Nature |
| | methanesulfonylpiperidin- | |
| | 3-yl)methyl]- | |
| | 5,6- | |
| | dimethylthieno[2,3- | |
| | d]pyrimidin-4-amine | |
| COc1ccccc1CCC(═O)NC1CCN(CC1)c1cc(nc(C)n1)C(F)(F)F | 3-(2- | Nature |
| | methoxyphenyl)-N- | |
| | {1-[2-methyl-6- | |
| | (trifluoromethyl)pyrimidin- | |
| | 4-yl]piperidin- | |
| | 4-yl}propa-mide | |
| CC(C)c1cc(Cl)ccc1OCC1CCNCC1 | 4-{[4-chloro-2- | Nature |
| | (propan-2- | |
| | yl)phenoxy]methyl} | |
| | piperidine | |
| | hydrochloride | |
| Cc1c(sc2nc[nH]c(═O)c12)C(═O)Nc1cccc(C)c1 | 5-methyl-N-(3- | Nature |
| | methylphenyl)-4- | |
| | oxo-3H,4H- | |
| | thieno[2,3- | |
| | d]pyrimidine-6- | |
| | carboxamide | |
| COc1ccc(\C═N\Nc2nc3sc(C)c(C)c3c(═O)[nH]2)cc1 | 2-[(E)-2-[(4- | Nature |
| | methoxyphenyl)methylidene] | |
| | hydrazin- | |
| | 1-yl]-5,6-dimethyl- | |
| | 3H,4H-thieno[2,3- | |
| | d]pyrimidin-4-one | |
| CCOc1ccccc1C(CC(O)═O)NC(C)═O | 3-acetamido-3-(2- | Nature |
| | ethoxyphenyl)propanoic | |
| | acid | |
| CC(C)CCOc1ccccc1C(═O)Nc1ccc(N)cc1C | N-(4-amino-2- | Nature |
| | methylphenyl)-2-(3- | |
| | methylbutoxy)benzamide | |
| CCc1ccc(OCC(═O)NC2CCN(CC2)C(C)c2ccccc2)cc1 | 2-(4-ethylphenoxy)- | Nature |
| | N-[1-(1- | |
| | phenylethyl)piperidin- | |
| | 4-yl]acetamide | |
| C(N1CCN(CC1)c1ncnc2sc3CCCc3c12)c1ccccc1 | 12-(4- | Nature |
| | benzylpiperazin-1- | |
| | yl)-7-thia-9,11- | |
| | diazatricyclo[6.4.0.0?, | |
| | ?]dodeca- | |
| | 1(12),2(6),8,10- | |
| | tetraene | |
| Cn1/c(═N/C(═O)[C@@]([C@](F)(F)F)([C@](F)(F)F)Br)cc([O−])n(c1═O)C | | Nature |
| Cn1/c(═N/C(═O)[C@]([C@](F)(F)F)([C@](F)(F)F)Cl)cc([O−])n(c1═O)C | | Nature |
| CC[C@@]1(C(═N)N(C(═O)N(C1═O)C)C)CC | | Nature |
| CC[C@@]1(C(═N)N(C(═O)N(C1═O)C)CC)CC | | Nature |
| CCC[C@]1(C(═N)N(C(═O)N(C1═O)C)C)CCC | | Nature |
| CCCN1C(═N)[C@](C(═O)N(C1═O)C)(CC)CC | | Nature |
| CC[C@@]1(/C(═N/[C@@H](C)C)NC(═O)N(C1═O)C)CC | | Nature |
| CCC[C@]1(C(═N)NC(═O)N(C1═O)C)CCC | | Nature |
| CCCC[C@@]1(C(═N)N(C(═O)N(C1═O)C)C)CCCC | | Nature |
| C[C@@H](C)CC[C@@]1(C(═[NH+])NC(═S)N═C1[O−])CC[C@H](C)C | | Nature |
| CCC/N═C/1[C@](C(═O)N(C(═O)N1)C)(CC)CC | | Nature |
| CCC[C@]1(/C(═N/C)NC(═O)N(C1═O)C)CCC | | Nature |
| CCC[C@]1(C(═[NH+])NC(═S)N═C1[O−])CCC | | Nature |
| CCCC/N═C/1[C@](C(═O)N(C(═O)N1)C)(CC)CC | | Nature |
| CCCC[C@@]1(C(═N)NC(═O)N(C1═O)C)CCCC | | Nature |
| CCCC[C@@]1(/C(═N/C)NC(═O)N(C1═O)C)CCCC | | Nature |
| CCN(CC)/C(═N\[S@](═O)(═O)c1ccc(cc1)C)/C(═C\1N(/C(═[N+]/[C@](C) | | Nature |
| (C)C)S1)C)C | | |
| c1cn(c(═O)nc1N)[C@H]1[C@@H]([C@@H]([C@H](O1)CO[P@](═O)([O−]) | | Nature |
| O[P@@](═O)([O−])[O−])O)O | | |
| CC[C@@]1(C(═NC(═O)N(C1═O)C)Nc1ccc(cc1)Cl)CC | | Nature |
| CC[C@@]1(/C(═N/c2ccccc2)N(C(═O)N(C1═O)C)C)CC | | Nature |
| c1cn(c(═O)nc1N)[C@H]1C[C@@H]([C@H](O1)CO[P@](═O)([O−]) | | Nature |
| O[P@@](═O)([O−])[O−])O | | |
| CC[C@@]1(C(═N)NC(═O)N(C1═O)C)c1ccccc1 | | Nature |
| CC[C@]1(C(═N)NC(═O)N(C1═O)C)c1ccccc1 | | Nature |
| c1cn(c(═O)nc1N)[C@@H]1[C@@H]([C@@H]([C@H](O1)CO)O)O | | Nature |
| c1cn(c(═O)nc1N)[C@H]1[C@@H]([C@@H]([C@H](O1)CO)O)O | | Nature |
| c1cn(c(═O)nc1N)[C@H]1[C@H]([C@@H]([C@H](O1)CO)O)O | | Nature |
| c1[n+]c(nc(═O)n1[C@H]1[C@@H]([C@@H]([C@H](O1)CO)O)O)N | | Nature |
| c1cn(c(═O)nc1N)[C@@H]1[C@H]([C@@H]([C@H](O1)CO)O)O | | Nature |
| CC[C@@]1(/C(═N/Cc2ccc(cc2)OC)NC(═O)N(C1═O)C)CC | | Nature |
| c1cn(c(═O)nc1N)[C@H]1[C@@H]([C@@H]([C@H](O)CO[P@](═O)([O−]) | | Nature |
| [O−])O)O | | |
| CC(═O)c1c(nc2n(c1═O)CCC2)N | | Nature |
| CC[C@@]1(C(═NC(═O)NC1═O)Nc1cccc(c1)C)CC | | Nature |
| CC[C@@]1(C(═NC(═O)NC1═O)Nc1ccccc1OC)CC | | Nature |
| c1cn(c(═O)nc1N)[C@H]1C[C@@H]([C@H](O1)CO[P@](═O)([O−])[O−])O | | Nature |
| CC/C(═N\c1cc([n+]c2c1cccc2)N(C)C)N(C)C | | Nature |
| CCC[C@]1(C(═NC(═O)NC1═O)Nc1cccc(c1)C)CCC | | Nature |
| c1cn(c(═O)nc1N)[C@@H]1C[C@H]([C@@H](O1)CO)O | | Nature |
| c1cn(c(═O)nc1N)[C@@H]1C[C@@H]([C@@H](O1)CO)O | | Nature |
| c1cn(c(═O)nc1N)[C@H]1C[C@@H]([C@@H](O1)CO)O | | Nature |
| c1cn(c(═O)nc1N)[C@H]1C[C@H]([C@@H](O1)CO)O | | Nature |
| c1cn(c(═O)nc1N)[C@H]1C[C@@H]([C@H](O1)CO)O | | Nature |
| CCC[C@]1(C(═NC(═O)NC1═O)Nc1ccc(cc1)OC)CCC | | Nature |
| C/N═C1\C/C(═[N+]/C)N([C@H](c2c1cccc2)O)C | | Nature |
| C/N═C1\C/C(═[N+]/C)N([C@@H](c2c1cccc2)O)C | | Nature |
| CCC[C@]1(C(═[N+]C(═O)NC1═O)N)CCC | | Nature |
| CCC[C@]1(C(═NC(═O)NC1═O)Nc1ccccc1)CCC | | Nature |
| CCCC[C@@]1(C(═NC(═O)NC1═O)Nc1ccccc1)CCCC | | Nature |
| Cc1cc(c(cc1)/N═C/[N+](═C/Nc1c(cc(cc1)C)C)C)C | | Nature |
| CN(C)c1cc2n(c(═O)n1)CCc1c2cc(c(c1)OC)OC | | Nature |
| CCCC[C@@]1(C(═[N+]C(═O)NC1═O)N)CCCC | | Nature |
| c1cn(c(═O)nc1N)[C@H]1[C@@H]([C@@H]([C@H](O1)CO[P@](═O)([O−])O[P@](═O)([O−]) | | Nature |
| O[P@](═O)([O−])[O−])O)O | | |
| c1cn(c(═O)nc1N)[C@H]1C[C@@H]([C@H](O1)CO[P@](═O)([O−])O[P@](═O)([O−]) | | Nature |
| O[P@](═O)([O−])[O−])O | | |
| C[N@@+](C)(C)CCO[P@](═O)([O−])O[P@@](═O)([O−]) | | Nature |
| OC[C@@H]1[C@H]([C@H]([C@@H](O1)n1ccc(nc1═O)N)O)O | | |
| CC[C@@H](C)Nc1cc2n(c(═O)n1)CCc1c2cc(c(c1)OC)OC | | Nature |
| CC[C@H](C)Nc1cc2n(c(═O)n1)CCc1c2cc(c(c1)OC)OC | | Nature |
| C[C@H](C)CNc1cc2n(c(═O)n1)CCc1c2cc(c(c1)OC)OC | | Nature |
| COc1cc2c(cc1OC)c1cc(nc(═O)n1CC2)N(CCO)CCO | | Nature |
| C[C@@H](C)CCCNc1cc2n(c(═O)n1)CCc1c2cc(c(c1)OC)OC | | Nature |
| CCCCCC[C@@H](C)Nc1cc2n(c(═O)n1)CCc1c2cc(c(c1)OC)OC | | Nature |
| CCCCCC[C@H](C)Nc1cc2n(c(═O)n1)CCc1c2cc(c(c1)OC)OC | | Nature |
| CCCCN(CCCC)c1cc2n(c(═O)n1)CCc1c2cc(c(c1)OC)OC | | Nature |
| CC(═O)NCCCCNc1cc2n(c(═O)n1)CCc1c2cc(c(c1)OC)OC | | Nature |
| CCC(═O)NCCCCCNc1cc2n(c(═O)n1)CCc1c2cc(c(c1)OC)OC | | Nature |
| C/C(═N\c1c2ccccc2[n+]c2c1CCN2C)N(C)C | | Nature |
| CC(═O)N(c1ccc(cc1)OC)c1cc2n(c(═O)n1)CCc1c2cc(c(c1)OC)OC | | Nature |
| CN(CC[C@H](CC(═O)N[C@H]1C═C[C@@H](O[C@@H]1C(═O)[O−]) | | Nature |
| n1ccc(nc1═O)N)[NH3+])C(═[NH2+])N | | |
| CN(CC[C@@H](CC(═O)N[C@H]1C═C[C@@H](O[C@@H]1C(═O)[O−]) | | Nature |
| n1ccc(nc1═O)N)[NH3+])C(═[NH2+])N | | |
| CN(CC[C@@H](CC(═O)N[C@@H]1C═C[C@@H](O[C@@H]1C(═O)[O−]) | | Nature |
| n1ccc(nc1═O)N)[NH3+])C(═[NH2+])N | | |
| CN(CC[C@H](CC(═O)N[C@@H]1C═C[C@@H](O[C@@H]1C(═O)[O−]) | | Nature |
| n1ccc(nc1═O)N)[NH3+])C(═[NH2+])N | | |
| CNc1cc2n(c(═O)n1)CCc1c2cc(c(c1)OC)OC | | Nature |
| COc1cc(cc(c1OC)OC)CNc1cc2n(c(═O)n1)CCc1c2cc(c(c1)OC)OC | | Nature |
| COc1cc2c(cc1OC)c1cc(nc(═O)n1CC2)NCCCO | | Nature |
| COc1cc2c(cc1OC)c1cc(nc(═O)n1CC2)NCCCC[NH3+] | | Nature |
| COCCCNc1cc2n(c(═O)n1)CCc1c2cc(c(c1)OC)OC | | Nature |
| CCOCCCNc1cc2n(c(═O)n1)CCc1c2cc(c(c1)OC)OC | | Nature |
| CCCCCCCNc1cc2n(c(═O)n1)CCc1c2cc(c(c1)OC)OC | | Nature |
| COc1cc2c(cc1OC)c1cc(nc(═O)n1CC2)NCCCCCC[NH3+] | | Nature |
| C[C@@H]1C[C@H](CN(C1)c1cc2n(c(═O)n1)CCc1c2cc(c(c1)OC)OC)C | | Nature |
| C[C@H]1C[C@@H](CN(C1)c1cc2n(c(═O)n1)CCc1c2cc(c(c1)OC)OC)C | | Nature |
| Cc1cc(cc(c1)Nc1cc2n(c(═O)n1)CCc1c2cc(c(c1)OC)OC)C | | Nature |
| Cc1ccc(c(c1)Nc1cc2n(c(═O)n1)CCc1c2cc(c(c1)OC)OC)C | | Nature |
| C[C@H](C)c1ccc(cc1)Nc1cc2n(c(═O)n1)CCc1c2cc(c(c1)OC)OC | | Nature |
| C[C@H](C)c1ccccc1Nc1cc2n(c(═O)n1)CCc1c2cc(c(c1)OC)OC | | Nature |
| Cc1cc(ccc1Nc1cc2n(c(═O)n1)CCc1c2cc(c(c1)OC)OC)OC | | Nature |
| COc1cc2c(cc1OC)c1cc(nc(═O)n1CC2)Nc1ccc(cc1)C(═O)[O−] | | Nature |
| COc1cc2c(cc1OC)c1cc(nc(═O)n1CC2)Nc1ccc(cc1)C(═O)OC | | Nature |
| CCOC(═O)[C@@H]1CCN(CC1)c1cc2n(c(═O)n1)CCc1c2cc(c(c1)OC)OC | | Nature |
| COc1cc(cc(c1)OC)Nc1cc2n(c(═O)n1)CCc1c2cc(c(c1)OC)OC | | Nature |
| CCOC(═O)[C@H]1CCCN(C1)c1cc2n(c(═O)n1)CCc1c2cc(c(c1)OC)OC | | Nature |
| CCOC(═O)[C@@H]1CCCN(C1)c1cc2n(c(═O)n1)CCc1c2cc(c(c1)OC)OC | | Nature |
| COc1ccc(c(c1)Nc1cc2n(c(═O)n1)CCc1c2cc(c(c1)OC)OC)OC | | Nature |
| CCC(═O)NCCCCCCNc1cc2n(c(═O)n1)CCc1c2cc(c(c1)OC)OC | | Nature |
| CCOC(═O)c1ccc(cc1)Nc1cc2n(c(═O)n1)CCc1c2cc(c(c1)OC)OC | | Nature |
| COc1ccc(cc1OC)CCNc1cc2n(c(═O)n1)CCc1c2cc(c(c1)OC)OC | | Nature |
| CCCCOC(═O)c1ccc(cc1)Nc1cc2n(c(═O)n1)CCc1c2cc(c(c1)OC)OC | | Nature |
| CN(C)/C(═N\c1cc[n+]cc1)c1ccccc1 | | Nature |
| CN(C)c1cc(c2ccccc2n1)/N═C(\c1ccccc1)N(C)C | | Nature |
| CN(C)c1cc(c2ccccc2[n+]1)/N═C(/Cc1ccccc1)N(C)C | | Nature |
| Cc1cc(c2ccccc2[n+]1)/N═C(\c1ccccc1)N(C)C | | Nature |
| C[C@H]1CCCN(C1)c1cc2n(c(═O)n1)CCc1c2cc(c(c1)OC)OC | | Nature |
| C[C@@H]1CCCN(C1)c1cc2n(c(═O)n1)CCc1c2cc(c(c1)OC)OC | | Nature |
| COc1cc2c(cc1OC)c1cc(nc(═O)n1CC2)Nc1ccccc1Br | | Nature |
| C[C@@H](c1ccccc1)Nc1cc2n(c(═O)n1)CCc1c2cc(c(c1)OC)OC | | Nature |
| C[C@H](c1ccccc1)Nc1cc2n(c(═O)n1)CCc1c2cc(c(c1)OC)OC | | Nature |
| COc1ccc(cc1)Nc1cc2n(c(═O)n1)CCc1c2cc(c(c1)OC)OC | | Nature |
| COc1cccc(c1)Nc1cc2n(c(═O)n1)CCc1c2cc(c(c1)OC)OC | | Nature |
| CCOc1ccccc1Nc1cc2n(c(═O)n1)CCc1c2cc(c(c1)OC)OC | | Nature |
| COc1ccc(cc1)CNc1cc2n(c(═O)n1)CCc1c2cc(c(c1)OC)OC | | Nature |
| C[C@@H](CCc1ccccc1)Nc1cc2n(c(═O)n1)CCc1c2cc(c(c1)OC)OC | | Nature |
| C[C@H](CCc1ccccc1)Nc1cc2n(c(═O)n1)CCc1c2cc(c(c1)OC)OC | | Nature |
| COc1ccc(cc1)CCNc1cc2n(c(═O)n1)CCc1c2cc(c(c1)OC)OC | | Nature |
| COc1cc2c(cc1OC)c1cc(nc(═O)n1CC2)Nc1cc(c(cc1C(═O)OC)OC)OC | | Nature |
| C[C@]1(Cc2c(cc3c(n2)n(cnc3═O)Cc2ccccc2)CO1)C | | Nature |
| CC(═O)N(c1ccc2c(c1)OCCO2)c1cc2n(c(═O)n1)CCc1c2cc(c(c1)OC)OC | | Nature |
| COc1cc2c(cc1OC)c1cc(nc(═O)n1CC2)NCc1ccccc1 | | Nature |
| COc1cc2c(cc1OC)c1cc(nc(═O)n1CC2)NCCc1ccccc1 | | Nature |
| C[C@H]1Cc2ccccc2N1c1cc2n(c(═O)n1)CCc1c2cc(c(c1)OC)OC | | Nature |
| C[C@@H]1Cc2ccccc2N1c1cc2n(c(═O)n1)CCc1c2cc(c(c1)OC)OC | | Nature |
| COc1cc2c(cc1OC)c1cc3nc4ccccc4c(═O)n3c(═O)n1CC2 | | Nature |
| Cc1ccc(cc1)[C@@H](CCN(c1cc2n(c(═O)n1)CCc1c2cc(c(c1)OC)OC)C(═O)C)c1ccco1 | | Nature |
| Cc1ccc(cc1)[C@H](CCN(c1cc2n(c(═O)n1)CCc1c2cc(c(c1)OC)OC)C(═O)C)c1ccco1 | | Nature |
| Cc1ccc(cc1)NC(═O)C1═C(CCC1)/[N+]═C\1CCCN1C | | Nature |
| CC[C@@H](C)[C@@]1(C(═N)N(C(═NC1═O)[O−])C)CC | | Nature |
| CC[C@H](C)[C@@]1(C(═N)N(C(═NC1═O)[O−])C)CC | | Nature |
| CC[C@@]1(C(═N)N(C(═NC1═O)[O−])C)CC | | Nature |
| C[C@@H](C)C[C@]1(C(═N)NC(═NC1═O)[O−])C[C@@H](C)C | | Nature |
| CC(═O)N(CC[C@@H](c1ccccc1)c1ccco1)c1cc2n(c(═O)n1)CCc1c2cc(c(c1)OC)OC | | Nature |
| CC(═O)N(CC[C@H](c1ccccc1)c1ccco1)c1cc2n(c(═O)n1)CCc1c2cc(c(c1)OC)OC | | Nature |
| CCC[C@@]1(C(═N)N(C(═NC1═O)[O−])C)CC | | Nature |
| CCC[C@]1(C(═N)N(C(═NC1═O)[O−])C)CC | | Nature |
| C[C@@H](C)CC[C@@]1(C(═N)N(C(═NC1═O)[O−])C)CC[C@H](C)C | | Nature |
| COc1cc2c(cc1OC)c1cc(nc(═O)n1CC2)NCc1ccc2c(c1)OCO2 | | Nature |
| COc1cc2c(cc1OC)c1cc(nc(═O)n1CC2)N1CCc2ccccc2C1 | | Nature |
| CCC[C@]1(C(═N)N(C(═NC1═O)[O−])C)CCC | | Nature |
| COc1cc2c(cc1OC)c1cc(nc(═O)n1CC2)n1ccc2c1cccc2 | | Nature |
| Cc1ccc(c(c1C#N)/N═C(\C)N(C)C)C | | Nature |
| CCCCN1C(═N)[C@](C(═NC1═O)[O−])(CC)CC | | Nature |
| CCCCC[C@@]1(C(═N)N(C(═NC1═O)[O−])C)CC | | Nature |
| CCCCC[C@]1(C(═N)N(C(═NC1═O)[O−])C)CC | | Nature |
| c1ccc2c(c1)c1c(c(═O)o2)[C@H](C2═C(O1)NC═N[C@H]2N)c1ccc(cc1)Br | | Nature |
| c1ccc2c(c1)c1c(c(═O)o2)[C@@H](C2═C(O1)NC═N[C@H]2N)c1ccc(cc1)Br | | Nature |
| c1ccc2c(c1)c1c(c(═O)o2)[C@H](C2═C(O1)NC═N[C@@H]2N)c1ccc(cc1)Br | | Nature |
| c1ccc2c(c1)c1c(c(═O)o2)[C@@H](C2═C(O1)NC═N[C@@H]2N)c1ccc(cc1)Br | | Nature |
| CCCC[C@@]1(C(═N)N(C(═NC1═O)[O−])C)CCCC | | Nature |
| C/C(═N\c1ccc(cc1C#N)Cl)N(C)C | | Nature |
| Cc1ccc(c(c1)C#N)/N═C(\C)N(C)C | | Nature |
| C/C(═N\c1ccccc1C#N)N(C)C | | Nature |
| COc1ccccc1[C@@H](CCNc1cc2n(c(═O)n1)CCc1c2cc(c(c1)OC)OC)c1ccco1 | | Nature |
| COc1ccccc1[C@H](CCNc1cc2n(c(═O)n1)CCc1c2cc(c(c1)OC)OC)c1ccco1 | | Nature |
| CCCCCC[C@@]1(C(═N)NC(═NC1═O)[O−])CCCCCC | | Nature |
| CC[C@@]1(/C(═N/CCc2ccc(c(c2)OC)OC)NC(═NC1═O)[O−])CC | | Nature |
| CC[C@@]1(/C(═N/[C@@H](C)c2ccccc2)NC(═NC1═O)[O−])CC | | Nature |
| CC[C@@]1(/C(═N/[C@H](C)c2ccccc2)NC(═NC1═O)[O−])CC | | Nature |
| CC[C@@]1(/C(═N/Cc2ccc(cc2)OC)NC(═NC1═O)[O−])CC | | Nature |
| CC[C@@]1(/C(═N/[C@@H](C)Cc2ccccc2)NC(═NC1═O)[O−])CC | | Nature |
| CC[C@@]1(/C(═N/[C@H](C)Cc2ccccc2)NC(═NC1═O)[O−])CC | | Nature |
| Cc1ccc(cc1)/C═C/1C(═NC(═O)N═C1[NH−])N | | Nature |
| CC[C@@]1(/C(═N/Cc2ccccc2)NC(═NC1═O)[O−])CC | | Nature |
| Cc1ccc(c(c1C#N)/N═C/1CCCCCN1C)C | | Nature |
| CC[C@@]1(/C(═N/CCc2ccccc2)NC(═NC1═O)[O−])CC | | Nature |
| c1ccc2c(c1)CN1Cc3ccccc3C1═[N+]C2═N | | Nature |
| CN1CCCC\1═N/c1ccc(cc1C#N)Cl | | Nature |
| CN1CCCCC\1═N/c1ccc(cc1C#N)Br | | Nature |
| CN1CCCCC\1═N/c1ccc(cc1C#N)Cl | | Nature |
| CN1CCCCCC\1═N/c1ccc(cc1C#N)Cl | | Nature |
| CN(C)/C(═N/c1ccccc1C#N)c1ccccc1 | | Nature |
| Cc1cc(c(c(c1)C#N)/N═C/1CCCN1Cc1ccccc1)C | | Nature |
| Cc1ccc(c(c1C#N)/N═C/1CCCN1Cc1ccccc1)C | | Nature |
| CN1C2═NNC═[N+][C@@]2(C(═O)N(C1═O)C)[O−] | | Nature |
| c1cnccc1c1ccn2c(c1)ncc1sc(═O)nc21 | | Nature |
| C[C@@]1([C@@H](O[C@]([C@@]([C@]1(C)C)(C)C)(C)C)N1[C@@H]2C(═C(N1)Br)C(═O)N | | Nature |
| C═N2)C | | |
| C[C@]1([C@H](O[C@@]([C@]([C@@]1(C)C)(C)C)(C)C)N1[C@@H]2C(═C(N1)Br)C(═O)NC═ | | Nature |
| N2)C | | |
| Cc1cc2c(cc1/N═C/N(C)C)nc1c(n2)c(═O)n(c(═O)n1C)C | | Nature |
| C[NH+](C)CCNc1cc2n(c(═O)n1)CCc1c2cc(c(c1)OC)OC | | Nature |
| CC[NH+](CC)CCNc1cc2n(c(═O)n1)CCc1c2cc(c(c1)OC)OC | | Nature |
| CC[NH+]1CCN(CC1)c1cc2n(c(═O)n1)CCc1c2cc(c(c1)OC)OC | | Nature |
| CN1C2═NNC═N[C@]2(C(═O)N(C1═O)C)O | | Nature |
| c1c2c([nH]n1)nc[nH]c2═O | | Nature |
| COc1cc2c(cc1OC)c1cc(nc(═O)n1CC2)N1CC[NH2+]CC1 | | Nature |
| CC(═O)OC[C@H]1[C@H]([C@@](CO1)(N1C═N[C@@H]2[C@@H]1N═CNC2═O)OC(═O)C)O | | Nature |
| C(═O)C | | |
| CC(═O)OC[C@@H]1[C@H]([C@@](CO1)(N1C═N[C@@H]2[C@@H]1N═CNC2═O)OC(═O)C) | | Nature |
| OC(═O)C | | |
| CC(═O)OC[C@@H]1[C@@H]([C@@](CO1)(N1C═N[C@@H]2[C@@H]1N═CNC2═O)OC(═O) | | Nature |
| C)OC(═O)C | | |
| CC(═O)OC[C@H]1[C@@H]([C@@](CO1)(N1C═N[C@@H]2[C@@H]1N═CNC2═O)OC(═O)C) | | Nature |
| OC(═O)C | | |
| C[C@H](C)[C@@]1(C(═O)N═C(N1)c1ccccc1[N+](═O)[O−])C | | Nature |
| C[C@H](C)[C@]1(C(═O)N═C(N1)c1ccccc1[N+](═O)[O−])C | | Nature |
| c1cc2cccc3c2c(c1)C(═N3)N1C[C@@H]2C[C@H](C1)c1cccc(═O)n1C2 | | Nature |
| c1ccc(cc1)Cc1nc2ccccc2c(═O)n1NC(═O)c1c(═O)c2cccc3c2n(c1[O−])CC3 | | Nature |
| CCNC(═O)[C@]12C(═NN═C[N−]1)N(C(═O)N2C)C | | Nature |
| CCNC(═O)[C@@]12C(═NN═C[N−]1)N(C(═O)N2C)C | | Nature |
| CCCNC(═O)[C@]12C(═NN═C[N−]1)N(C(═O)N2C)C | | Nature |
| CCCNC(═O)[C@@]12C(═NN═C[N−]1)N(C(═O)N2C)C | | Nature |
| CCOC(═O)/C(═N/Nc1ccc(cc1)C)n1c(cc(nn(c1═S)C)C)[S−] | | Nature |
| CCCCNC(═O)[C@]12C(═NN═C[N−]1)N(C(═O)N2C)C | | Nature |
| CCCCNC(═O)[C@@]12C(═NN═C[N−]1)N(C(═O)N2C)C | | Nature |
| CCOC(═O)/C(═N/Nc1ccccc1)n1c(cc(nn(c1═S)C)C)[S−] | | Nature |
| c1c(nc(═O)n(n1)[C@H]1[C@@H]([C@@H]([C@@H](O1)CO)O)O)NCC(═O)N | | Nature |
| CCOC(═O)c1nn(c2cc(nn(c(═S)n12)C)C)c1ccc(cc1)C | | Nature |
| CCC[C@@]1(/C(═N/c2cccnc2)NC(═NC1═O)[O−])CCC | | Nature |
| C[N+]1═C2C(═[N+]C(═N)N═C2[O−]) | | Nature |
| NC[C@@H]1CNc1ccc(cc1)C(═O)N[C@@H](CCC(═O)O)C(═O)[O−] | | |
| Cc1c(nc(═O)n(n1)[C@@H]1CCCO1)N | | Nature |
| CCOC(═O)c1nn(c2cc(nn(c(═S)n12)C)C)c1ccccc1 | | Nature |
| c1ccc(cc1)C(═O)OC[C@@H]1[C@H]([C@H]([C@@H](O1)n1c(═O)nc(cn1)NCC(═O)N)O)O | | Nature |
| c1c(nc(═O)n(n1)[C@H]1CCCO1)N | | Nature |
| c1c(nc(═O)n(n1)[C@@H]1CCCO1)N | | Nature |
| C[C@]1(C(═[N+]([O−])C(═N1)c1cccnc1)c1ccccc1)C | | Nature |
| c1cc(ccc1C(═O)N[C@@H](CCC(═O)O)C(═O)[O−])NC[C@@H]1CNC2═[N+]C(═N)N═C([O−]) | | Nature |
| C2═[N+]1C═O | | |
| c1cc2cccc3c2c(c1)/c(═N/C(═O)c1ccc(cc1[N+](═O)[O−])[N+](═O)[O−])[nH]3 | | Nature |
| c1c2c(ncn2)[nH]c(═O)[nH]1 | | Nature |
| c1ccc(cc1)/C(═N/OCO/N═C(/C1═CC═CCC1)N)N | | Nature |
| c1ccc(cc1)C1═NN2CNC(═N)[N+]═C2CC1 | | Nature |
| Cc1cc(no1)/[N+]═C/1c2ccccc2CN1c1cc(on1)C | | Nature |
| C1═NC(═O)C2═NC═[N+](C2═N1)[C@H]1[C@@H]([C@@H]([C@H](O1)CO)O)O | | Nature |
| c1[n−]c2c(═[NH+])ncn(c2n1)C[C@@H](CO)O | | Nature |
| c1nc2c(n1[C@H]1C[C@@H]([C@@H](O1)CO)O)N═CNC[C@@H]2O | | Nature |
| c1nc2c(n1[C@@H]1C[C@@H]([C@@H](O1)CO)O)N═CNC[C@@H]2O | | Nature |
| c1cc(ccc1C(═O)N[C@@H](CCC(═O)O)C(═O)[O−])NC[C@H]1CNC2═[N+]C(═N)N═C([O−]) | | Nature |
| C2═N1 | | |
| Cn1C(═O)c2nc(nn(c2nc1═O)C)c1ccc(cc1)OC | | Nature |
| CCn1c2nc(═O)n(c(═O)c2nc(n1)c1ccc(cc1)OC)C | | Nature |
| c1[nH]c2c(n1)c(═S)[nH]cn2 | | Nature |
| c1nc2[nH]cnc2c(═O)[nH]1 | | Nature |
| Cn1c(═O)c2nc(nn(c2nc1═O)C)c1ccccc1 | | Nature |
| CCn1c2nc(═O)n(c(═O)c2nc(n1)c1cccs1)C | | Nature |
| CCn1c2nc(═O)n(c(═O)c2nc(n1)c1cccnc1)C | | Nature |
| CCn1c2nc(═O)n(c(═O)c2nc(n1)/C═C/c1ccccc1)C | | Nature |
| c1nc2c(n1[C@@H]1C[C@H]([C@@H](O1)CO)O)NC═[N+]C[C@@H]2O | | Nature |
| c1nc2c(n1[C@H]1C[C@H]([C@@H](O1)CO)O)NC═[N+]C[C@@H]2O | | Nature |
| CCOC(═O)/C(═N/Nc1ccc(cc1)Cl)n1c(cc(nn(c1═S)C)C)[S−] | | Nature |
| CCOC(═O)c1nn(c2cc(nn(c(═S)n12)C)C)c1ccc(cc1)Cl | | Nature |
| Cc1c2c(c(c(c1OC)C/C═C(\C)CCC(═O)[O−])O)C(═O)OC2 | | Nature |
| Cc1cc(c(c(c1C(═O)[O−])O)C)O | | Nature |
| CCOC(═O)c1c(cc(cc1O)O)C | | Nature |
| Cc1cc(cc(c1C(═O)OC)O)O | | Nature |
| Cc1cc(c(c(c1C(═O)Oc1cc(c(c(c1C)O)C(═O)OC)C)O)C═O)O | | Nature |
| Cc1cc(cc(c1C(═O)Oc1cc(c(c(c1)O)C(═O)[O−])C)O)O | | Nature |
| CCCCCc1cc(c(c(c1C(═O)[O−])O)[C@H]1C═C(CC[C@@H]1C(═C)C)C)O | | Nature |
| Cc1cc(c(c2c1C(═O)Oc1c(c(c3c(c1O2)[C@@H](OC3═O)O)O)C)C═O)OC | | Nature |
| Cc1cc(cc(c1C(═O)Oc1cc(c(c(c1)O)C(═O)Oc1cc(c(c(c1)O)C(═O)[O−])C)C)O)O | | Nature |
| CCCCCc1cc2cc(cc(c2c(═O)o1)Oc1c(cc(c(c1CCCCC)C(═O)[O−])O)O)O | | Nature |
| Cc1cc(c(c(c1C(═O)[O−])O)C)OC(═O)c1c(cc(c(c1OC)C)OC)C | | Nature |
| Cc1cc(cc(c1C(═O)Oc1cc(c(c(c1)O)C(═O)Oc1cc(c(c(c1)O)C(═O)[O−])C)C)O)O | | Nature |
| COc1ccc(c(c1OC)O)C(═O)[O−] | | Nature |
| COc1cc(c(c(c1)O)C(═O)[O−])O | | Nature |
| C[C@@H]1CCCC(═O)CCCC═Cc2cc(cc(c2C(═O)O1)O)O | | Nature |
| C1CCc2c(c3c(nc(nc3s2)SCCC(═O)N)N)C1 | | Nature |
| COC(═O)CCSc1nc(c2c3c(sc2n1)CCCC3)N | | Nature |
| C1CCc2c(c3c(s2)[n+]c(sc3═O)N)C1 | | Nature |
| C1CCc2c(c3c(nc(nc3s2)SCCC(═O)N)N)C1 | | Nature |
| COC(═O)CCSc1nc(c2c3c(sc2n1)CCCC3)N | | Nature |
| Cn1c2c(c3c(s2)CCCC3)c(═O)n(c1═O)c1cccc(c1)Cl | | Nature |
| CCOC(═O)c1c(nc2c(c1N)c1c(s2)CCCC1)C | | Nature |
| Cc1nc2c(c(cs2)c2ccccc2)c(═O)n1NC(═O)c1ccccc1Cl | | Nature |
| Cc1c2c(nc3n(c2═O)CCC3)sc1C(═O)NC[C@@H]1CCC[NH+]2[C@@H]1CCCC2 | | Nature |
| n1c(cn(c1)C)C[C@@H](C(═O)O)N | | Nature |
| Cn1cncc1C[C@@H](C(═O)[O−])[NH3+] | | Nature |
| c1(c(ncn1C)S)C[C@@H](C(═O)O)N | | Nature |
| c1(c(n(cn1)C)S)C[C@@H](C(═O)O)N | | Nature |
| C[N@@+](C)(C)[C@@H](Cc1c[nH]c(═S)[nH]1)C(═O)[O−] | | Nature |
| C[N@@+](C)(C)[C@H](Cc1c[nH]c(═S)[nH]1)C(═O)[O−] | | Nature |
| c1(c(ncn1C)S)C[C@@H](C(═O)O)N(C)C | | Nature |
| c1(n(cnc1)C)C[C@@H](C(═O)O)NC(═O)CCN | | Nature |
| c1(n(cnc1)C)C[C@@H](C(═O)O)NC(═O)CCCN | | Nature |
| n1cn(c(c1)CC(═O)O)C | | Nature |
| Cn1c2c(c3c(s2)CCCC3)c(═O)n(c1═O)c1ccccc1 | | Nature |
| COC(═O)CCSc1nc(c2c3c(sc2n1)CCCC3)N | | Nature |
| Cn1c2c(c3c(s2)CCCC3)c(═O)n(c1═O)c1cccc(c1)Cl | | Nature |
| C[C@@H](C)COc1ccccc1C(═O)NCCC(═O)[O−] | | Nature |
| N1S(═O)(═O)N(C(═O)c2c1cccc2)C(C)C | | Nature |
| CCOC(═O)[C@H]1C(═O)N[C@@H]2[C@@]31CCCC[C@@H]3Oc1c2cccc1 | | Nature |
| CC[C@H]1CN(CC[C@H]1CC(═O)NCc1ccccc1OC)C(═O)Nc1cccc(c1)C(═O)OCC | | Nature |
| c1ccc(cc1)COc1ccc2c(c1)ccn2CCC(═O)NCCC(═O)[O−] | | Nature |
| c1(c(cc(c(c1)Cl)N)OC)C(═O)N[C@H]1[C@H](CN(CC1)CCCOc1ccc(cc1)F)OC | | Nature |
| c12c(C(═O)N[C@H](O1)CC(═O)O)cccc2 | | Nature |
| COc1ccc(cc1N)C(═O)NCCC(═O)[O−] | | Nature |
| C[C@@H](C)COc1ccc(cc1)C(═O)NCCC(═O)[O−] | | Nature |
| CCOC(═O)[C@@H]1C(═O)N[C@H]2[C@@]31CCCC[C@H]3Oc1c2cccc1 | | Nature |
| CCC(═O)N1CC[C@]2(CCCC[C@H]2[C@H]1c1ccccc1OCC)O | | Nature |
| CCCCCOc1ccc(cc1)C(═O)N1CC[C@]2(CCCC[C@H]2C1)O | | Nature |
| CCOc1ccccc1[C@@H]1[C@H]2CCCC[C@@]2(CCN1C(═O)C)O | | Nature |
| c1ccc(cc1)COc1ccc2ccn(c2c1)CCC(═O)N1CCC[C@@H](C1)C(═O)[O−] | | Nature |
| CCCC(═O)N1CC[C@]2(CCCC[C@H]2[C@@H]1c1ccccc1OC)O | | Nature |
| c12[C@@H](c3ccccc3)CC(═O)Oc1cc(c(c2)O)OC | | Nature |
| [C@H]1(c2c(cc(cc2O)OC)OC(═O)C1)c1ccc(cc1)OC | | Nature |
| [C@H]1(c2c(cc(cc2O)OC)OC(═O)C1)c1ccc(cc1)O | | Nature |
| COc1ccc(cc1)[C@H]1CC(═O)Oc2c1c(cc(c2)O)O | | Nature |
| COc1ccc(cc1)[C@@H]1CC(═O)Oc2c1c(cc(c2)O)O | | Nature |
| [C@H]1(c2c(cc(c(c2)CCc2cc(c(cc2)OC)OC)O)OC(═O)C1)c1cc(c(cc1)O)O | | Nature |
| c12c3c(c4c(c1CC[C@](O2)(C)C)O[C@@](CC4)(C)C)OC(═O)C[C@H]3c1cc(c(cc1)O)O | | Nature |
| c12c(cc(c(c1)OC)O)CCC(═O)O2 | | Nature |
| C[C@H]1CC(═O)Oc2c1ccc(c2)O | | Nature |
| C[C@@H]1CC(═O)Oc2c1ccc(c2)O | | Nature |
| [C@H]1([C@H]2[C@H](c3c(OC2═O)cc(cc3OC)O)c2ccc(cc2)O)[C@H](c2c(OC1═O)cc(cc2OC) | | Nature |
| O)c1ccc(cc1)O | | |
| c1cc(ccc1[C@@H]1c2c(cc(cc2OC(═O)[C@@H]1[C@@H]1[C@@H](c2c(c(c(c(c2OC1═O)O)O) | | Nature |
| O)O)c1ccc(cc1)O)O)O)O | | |
| c1cc(ccc1[C@H]1c2c(cc(cc2OC(═O)[C@@H]1[C@@H]1[C@@H](c2c(c(c(c(c2OC1═O)O)O)O) | | Nature |
| O)c1ccc(cc1)O)O)O)O | | |
| c1cc(ccc1[C@@H]1c2c(cc(cc2OC(═O)[C@H]1[C@@H]1[C@@H](c2c(c(c(c(c2OC1═O)O)O)O) | | Nature |
| O)c1ccc(cc1)O)O)O)O | | |
| c1cc(ccc1[C@H]1c2c(cc(cc2OC(═O)[C@H]1[C@@H]1[C@@H](c2c(c(c(c(c2OC1═O)O)O)O)O) | | Nature |
| c1ccc(cc1)O)O)O)O | | |
| c12[C@@H](c3ccccc3)CC(═O)Oc1cc(c(c2)O)OC | | Nature |
| [C@H]1(c2c(cc(cc2O)OC)OC(═O)C1)c1ccc(cc1)OC | | Nature |
| [C@H]1(c2c(cc(cc2O)OC)OC(═O)C1)c1ccc(cc1)O | | Nature |
| COc1ccc(cc1)[C@H]1CC(═O)Oc2c1c(cc(c2)O)O | | Nature |
| COc1ccc(cc1)[C@@H]1CC(═O)Oc2c1c(cc(c2)O)O | | Nature |
| [C@H]1(c2c(cc(c(c2)CCc2cc(c(cc2)OC)OC)O)OC(═O)C1)c1cc(c(cc1)O)O | | Nature |
| c12c3c(c4c(c1CC[C@](O2)(C)C)O[C@@](CC4)(C)C)OC(═O)C[C@H]3c1cc(c(cc1)O)O | | Nature |
| c12c(cc(c(c1)OC)O)CCC(═O)O2 | | Nature |
| C[C@H]1CC(═O)Oc2c1ccc(c2)O | | Nature |
| C[C@@H]1CC(═O)Oc2c1ccc(c2)O | | Nature |
| [C@H]1([C@H]2[C@H](c3c(OC2═O)cc(cc3OC)O)c2ccc(cc2)O)[C@H](c2c(OC1═O)cc(cc2OC) | | Nature |
| O)c1ccc(cc1)O | | |
| c1cc(ccc1[C@@H]1c2c(cc(cc2OC(═O)[C@@H]1[C@@H]1[C@@H](c2c(c(c(c(c2OC1═O)O)O) | | Nature |
| O)O)c1ccc(cc1)O)O)O)O | | |
| c1cc(ccc1[C@H]1c2c(cc(cc2OC(═O)[C@@H]1[C@@H]1[C@@H](c2c(c(c(c(c2OC1═O)O)O)O) | | Nature |
| O)c1ccc(cc1)O)O)O)O | | |
| c1cc(ccc1[C@@H]1c2c(cc(cc2OC(═O)[C@H]1[C@@H]1[C@@H](c2c(c(c(c(c2OC1═O)O)O)O) | | Nature |
| O)c1ccc(cc1)O)O)O)O | | |
| c1cc(ccc1[C@H]1c2c(cc(cc2OC(═O)[C@H]1[C@@H]1[C@@H](c2c(c(c(c(c2OC1═O)O)O)O)O) | | Nature |
| c1ccc(cc1)O)O)O)O | | |
| COc1ccc(cc1O)[C@H]1CC(═O)c2c(cc(cc2O1)O)O | | Nature |
| COc1ccc(cc1O)[C@@H]1CC(═O)c2c(cc(cc2O1)O)O | | Nature |
| COc1ccc(cc1O)[C@@H]1CC(═O)c2c(cc(cc2O1)OC)O | | Nature |
| Cc1cc(c2c(c1)O[C@@H](CC2═O)c1ccc(c(c1)O)OC)O | | Nature |
| COc1ccc(cc1OC)[C@H]1CC(═O)c2c(cc(cc2O1)O)O | | Nature |
| COc1cc(ccc1O)[C@H]1CC(═O)c2c(cc(cc2O1)O)O | | Nature |
| COc1cc(c2c(c1)O[C@@H](CC2═O)c1ccc(c(c1)O)O)O | | Nature |
| c1cc(c(cc1[C@H]1CC(═O)c2c(cc(cc2O1)O)O)O)O | | Nature |
| c12C(═O)C[C@H](Oc1cc(cc2O)O)c1cc(c(c(c1)O)O)O | | Nature |
| c1cc(c(cc1C1CC(═O)c2c(cc(cc2O1)O)O)O)O | | Nature |
| COc1cc(cc(c1OC)OC)[C@H]1CC(═O)c2c(cc(cc2O1)O)O | | Nature |
| Cc1cc(c2c(c1)O[C@H](CC2═O)c1ccc(c(c1)O)OC)O | | Nature |
| COc1cc(c2c(c1)O[C@H](CC2═O)c1ccc(c(c1)OC)O)O | | Nature |
| COc1ccc(cc1OC)[C@H]1CC(═O)c2c(cc(cc2O1)OC)O | | Nature |
| c1cc(cc(c1)O)O | | Nature |
| c1c(cc(cc1O)O)O | | Nature |
| O.Oc1cc(O)cc(c1)O | | Nature |
| c1cc2cc3cccc(c3c(c2c(c1)O)O)O | | Nature |
| c1ccc2c(c1)cc(cc2O)O | | Nature |
| c1cc(c(c(c1)O)O)O | | Nature |
| c1cc2cc(cc(c2c(c1)O)O)O | | Nature |
| c12c(c(cc(c1)O)O)ccc(c2)O | | Nature |
| c12c(cc(cc1ccc1c2c(O)ccc1)O)O | | Nature |
| c1(c(ccc(c1)O)O)O | | Nature |
| c1cc2cc(ccc2c(c1)O)O | | Nature |
| c1cc(ccc1O)O | | Nature |
| c1(c(c2c(cc1O)cccc2)O)O | | Nature |
| c1ccc2cc(ccc2c1)O | | Nature |
| COc1ccc(cc1O)[C@H]1CCc2ccc(cc2O1)O | | Nature |
| c12c(CC[C@H](O1)c1cc(c(cc1)OC)O)ccc(c2)O | | Nature |
| Cc1c(ccc2c1O[C@@H](CC2)c1ccc(c(c1)O)OC)O | | Nature |
| [C@H]1(c2cc(c(cc2)O)OC)Oc2c(CC1)ccc(c2)OC | | Nature |
| COc1cc(ccc1O)[C@H]1CCc2ccc(cc2O1)O | | Nature |
| [C@H]1(c2cc(c(cc2)O)OC)Oc2c(CC1)ccc(c2)O | | Nature |
| [C@H]1(c2cc(c(cc2)O)O)Oc2c(CC1)ccc(c2)OC | | Nature |
| [C@H]1(c2cc(c(cc2)O)O)Oc2c(CC1)ccc(c2)OC | | Nature |
| Cc1c(ccc2c1O[C@H](CC2)c1ccc(c(c1)O)OC)O | | Nature |
| c12c(c(cc(c2)OC)O)CC[C@H](O1)c1cc(c(cc1)O)OC | | Nature |
| c1cc(c(cc1[C@H]1CCc2c(cc(cc2O1)O)O)O)O | | Nature |
| c12c(O[C@@H](CC2)c2cc(c(cc2)O)OC)cc(cc1OC)OC | | Nature |
| c12c(c(ccc1CC[C@H](O2)c1cc(c(c(c1)O)OC)OC)OC)OC | | Nature |
| COc1cc(ccc1O)[C@H]1CCc2cc(c(cc2O1)O)OC | | Nature |
| c12c(O[C@@H](CC2)c2cc(c(cc2)O)O)cc(cc1OC)OC | | Nature |
| COc1ccc(cc1OC)CC(═O)c1ccc(cc1O)O | | Nature |
| COc1ccc(cc1OC)CC(═O)c1c(cc(cc1O)O)O | | Nature |
| C[C@@H](c1ccc(c(c1)O)OC)C(═O)c1ccc(cc1O)O | | Nature |
| C[C@H](c1ccc(c(c1)O)OC)C(═O)c1ccc(cc1O)O | | Nature |
| C[C@@H](c1cc(cc(c1)OC)OC)C(═O)c1ccc(cc1O)O | | Nature |
| COc1ccc(cc1)CC(═O)c1c(cc(cc1O)O)O | | Nature |
| COc1ccc(cc1)CC(═O)c1ccc(cc1O)O | | Nature |
| COc1ccc(c(c1)OC)C(═O)Cc1ccc(c(c1)OC)OC | | Nature |
| CCOC(═O)CC(O)(CC(═O)OCC)C(═O)OCC | Triethyl citrate | VCF |
| OC(═O)C(═O)CC1═CC═CC═C1 | Phenylpyruvic | VCF |
| | acid | |
| CC(═O)CCC1═CC═C(O)C═C1 | 4-(4- | VCF |
| | Hydroxyphenyl)- | |
| | 2-butanone | |
| CCOCC═C(C)C | 1-Ethoxy-3- | |
| | methyl-2-butene | |
| CC(C)═CCC(O)C1═CC(═O)C2═C(C(O)═CC═C2O)C1═O | shikonin | |
| COC1═CC═C(C═C1O)[C@@H]1CC(═O)C2═C(O1)C═C(O[C@@H]1O[C@H] | hesperidin | |
| (CO[C@@H]3O[C@@H](C)[C@H](O)[C@@H](O)[C@H]3O)[C@@H](O)[C | | |
| @H](O)[C@H]1O)C═C2O | | |
| OC1CSC(O)CS1 | 1,4-Dithiane-2,5- | |
| | diol | |
| CC(C)CC(═O)OCCCC1═CC═CC═C1 | 3-Phenylpropyl | |
| | isovalerate | |
| CC1CCC(CC1)C(C)(C)OC(C)═O | dihydro-alpha- | VCF |
| | terpinyl acetate | |
| CCC1═C(O)C(O)═CC═C1 | 3-Ethyl-1,2- | VCF |
| | benzenediol | |
| OC1═C(O)C═CC═C1 | catechol | VCF |
| CCOC(═O)C(C)(OCC)OCC | ethyl pyruvate | VCF |
| | acetal | |
| CCCC[C@H]1OC(═O)C[C@H]1C | cis-oak lactone | VCF |
| CCCCCCC1═CC═CC═N1 | 2-hexyl pyridine | VCF |
| COC1═CC(\C═C\C(\O)═C\C(═O)\C═C\C2═CC(OC)═C(O)C═C2)═CC═C1O | curcumin | PMN |
| OC1═CC2═C(C(O)═C1)C(═O)C═C(O2)C1═CC(O)═C(O)C═C1 | eriodictyol | PMN |
| CC1CC2(CCCCC2)OC═C1 | herbal undecanol | PMN |
| C\C1═C/CCC2(C)OC2CC\C(C)═C\CC1 | (E)-woody | PMN |
| | epoxide | |
| CN1C2CCC1(CC(C2)OC(═O)CC(C)═S)C═O | tropical 3- | PMN |
| | thiobutyrate | |
| COC1═CC═C(C═C1)C(═O)OCC(O)═O | 4- | PMN |
| | methoxybenzoyl | |
| | oxyacetic acid | |
| CCCCCC(O)CC(═O)CCC1CCC(O)C(C1)OC | gingerol | PMN |
| CCOC(═O)\C═C\C1═CC═C(O)C(OC)═C1 | ethyl 4-hydroxy- | VCF |
| | 3-methoxy- | |
| | cinnamate | |
| O═CN1CCCCC1 | 1- | VCF |
| | Piperidinecarboxaldehyde | |
| CC1(C)C2CC1C(═C)C(═O)C2 | (−)-Pinocarvone | VCF |
| CC(═O)OC1═CC═C(O)C═C1 | 4-Hydroxyphenyl | VCF |
| | acetate | |
| NCCCCNC(N)═N | Agmatine | VCF |
| COC1═CC═C(\C═C\C═C)C═C1OC | \(E)-4-(1,3- | VCF |
| | butadienyl)-1,2- | |
| | dimethoxybenzene\ | |
| CC(C)OC1═CC═C(C)C═C1 | 1-isopropoxy-4- | VCF |
| | methylbenzene | |
| O═CC1═NC═CN1 | 2- | VCF |
| | imidazolecarbaldehyde | |
| CCN1CCCCC1 | 1-ethylpiperidine | VCF |
| CCOC(═O)COC | ethyl | VCF |
| | methoxyacetate | |
| COC(═O)CO | methyl | VCF |
| | hydroxyacetate | |
| CCOC(═O)C\C═C\C1═CC═CC═C1 | ethyl 4-phenyl-3- | VCF |
| | butenoate | |
| CNCC(O)═O | Sarcosine | CosIng |
| CCCCCCCCCCCCCCC[C@@H](O)[C@@H](N)CO | Sphinganine | CosIng |
| CC(═O)N[C@@H](CCC(O)═O)C(O)═O | N-acetyl-L- | CosIng |
| | glutamic acid | |
| CC(C)(C)[N+]([O−])═CC1═CC═[N+]([O−])C═C1 | PYRIDYLOXIDET- | CosIng |
| | BUTYLNITRONE | |
| OC[C@H](O)[C@H]1OC(═O)C(O)═C1OC1O[C@H](CO)[C@@H](O)[C@H] | ASCORBYL | CosIng |
| (O)[C@H]1O | GLUCOSIDE | |
| C\C(═C/CN(C═O)[C@@H](CC(N)═O)C(O)═O)\C═C\C═C(/C)\C═C\C1═C(C)CCCC1(C)C | RETINYL | CosIng |
| | FORMYL | |
| | ASPARTAMATE | |
| CCCOCCC(O)O | PROPOXYPROP | CosIng |
| | ANEDIOL | |
| CCCCCCCCCCCCCCCCCC(═O)OC1═C(O)C(═O)O[C@@H]1[C@@H](O) | ASCORBYL | CosIng |
| CO | STEARATE | |
| CC1═CC═CC═C1NC(═N)NC(N)═N | O-TOLYL | CosIng |
| | BIGUANIDE | |
| CCCCCC[C@@H](O)C\C═C/CCCCCCCC(═O)OCC(COC(═O)CCCCCCC\C═ | TRIRICINOLEIN | CosIng |
| C/C[C@H](O)CCCCCC)OC(═O)CCCCCCC\C═C/C[C@H](O)CCCCCC | | |
| CCCCCCCCCCCCCCCCCCOC(═O)C(CC1═CC═C(O)C═C1)(C(C)(C)C)C(C) | OCTADECYL DI- | CosIng |
| (C)C | T-BUTYL-4-HYDROXY- | |
| | HYDROCINNAMATE | |
| OC(═O)C1═CC═CC═C1S | THIOSALICYLIC | CosIng |
| | ACID | |
| OC1═C(O)C(═CC═C1)C1═CC═CC═C1 | DIHYDROXYBIPHENYL | CosIng |
| NC(CCCC═O)C(O)═O | Allysine | FooDB |
| OC(═O)C(═C)OP(O)(O)═O | Phosphoenolpyruvic | FooDB |
| | acid | |
| OCC1OC(CO)(OCC2(OCC3(OC4OC(CO)C(O)C(O)C4O)OC(CO)C(O)C3O) | Nystose | FooDB |
| OC(CO)C(O)C2O)C(O)C1O | | |
| CC(C)═CCCC(C)(OC1OC(COC2OCC(O)C(O)C2O)C(O)C(O)C1O)C═C | L-Linalool 3- | FooDB |
| | [xylosyl-(1->6)- | |
| | glucoside] | |
| CC1CCC2(CCC3(C)C(═CCC4C5(C)CCC(O)C(C)(C)C5CCC34C)C2C1C)C | Carissic acid | FooDB |
| (O)═O | | |
| CCCCCC(O)CC(═O)CCC1═CC═C(O)C(OC)═C1 | Gingerol | FooDB |
| CC(C)(OC1OC(CO)C(O)C(O)C1O)C1CCC(CO)(O1)C═C | cis-10- | FooDB |
| | Hydroxylinalyl | |
| | oxide 7-glucoside | |
| CC1═C(O)C(═N)CC1 | 5-Imino-2-methyl- | FooDB |
| | 1-cyclopenten-1- | |
| | ol | |
| NCCCCCNC(N)═N | Homoagmatine | FooDB |
| CC(C)═CCC1═C2OC═C(C(═O)C2═C(O)C═C1O)C1═C(O)C═C(O)C═C1 | 2′,4′,5,7- | FooDB |
| | Tetrahydroxy-8- | |
| | prenylisoflavone | |
| COC1═C(O)C2═C(C═C1)C1COC3═C(C═CC(OC)═C3O)C1O2 | Melilotocarpan D | FooDB |
| CCCCCCCC1OC1CC#CC#CC(CC)OC(C)═O | Ginsenoyne G | FooDB |
| COC1═CC(OC)═C(OC)C2═C1C(═O)C1═C(O2)C(OC)═C2OC(C)(C)C═CC2═ | Dulxanthone G | FooDB |
| C1O | | |
| CC(O)C1OC(═O)C═C1 | L-erythro-5-(1- | FooDB |
| | Hydroxyethyl)- | |
| | 2(5H)-furanone | |
| CC(═O)C1═C2NC(═O)OC2═CC═C1 | 4-Acetyl-2(3H)- | FooDB |
| | benzoxazolone | |
| COC1═C(OC)C═C(C═C1)C1OCC2(O)C1COC2C1═CC(OC)═C(O)C═C1 | 8-Hydroxy-4′- | FooDB |
| | methoxypinoresinol | |
| COC1═CC═C(C═C1)C1═CC(═O)C2═C(O1)C(OC)═C(OC)C═C2O | 5-Hydroxy-4′,7,8- | FooDB |
| | trimethoxyflavone | |
| CC(CC(═O)\C═C(\C)C1CC(O)C2(C)C3═C(C(═O)CC12C)C1(C)CCC(═O)C(C) | Ganoderenic acid A | FooDB |
| (C)C1CC3O)C(O)═O | | |
| COC1═CC(═O)C2═C(C═C(C)C(C(C)═O)═C2O)C1═O | 2- | FooDB |
| | Methoxystypandrone | |
| CC(CC(═O)\C═C(\C)C1CC(O)C2(C)C3═C(C(═O)CC12C)C1(C)CCC(O)C(C) | Ganoderenic acid C | FooDB |
| (C)C1CC3O)C(O)═O | | |
| CC(═C)C1CC2═C(OC3═C(C(O)═CC4═C3C═CC(C)(C)O4)C2═O)C2═C1C(O)═ | Artonol C | FooDB |
| C1OC(C)(C)C═CC1═C2O | | |
| CC(OC1OC(COC(C)═O)C(OC2OC(CO)C(O)C(O)C2O)C(O)C1O)C1CC(O)C | (15a,20R)- | FooDB |
| 2C3CCC4═CC(═O)CCC4(C)C3CCC12C | Dihydroxypregn- | |
| | 4-en-3-one 20- | |
| | [glucosyl-(1->4)- | |
| | 6-acetyl- | |
| | glucoside] | |
| CC1═COC2═C1CC1C(═C)CCC(═O)C1(C)C2 | 8,12-Epoxy- | FooDB |
| | 4(15)7,11- | |
| | eudesmatrien-1- | |
| | one | |
| CC(═C)C1CC2═CC(C(═O)CO)═C(O)C═C2O1 | (S)-2,3-Dihydro- | FooDB |
| | 6-hydroxy-5- | |
| | (hydroxyacetyl)- | |
| | 2- | |
| | isopropenylbenzofuran | |
| CC1═C(O)C(C)═C2OC(CC(═O)C2═C1O)C1═CC═CC═C1 | (S)-5,7- | FooDB |
| | Dihydroxy-6,8- | |
| | dimethylflavanone | |
| COC1═CC═CC2═C1C(CNC(═S)SC)═CN2 | 4- | FooDB |
| | Methoxybrassinin | |
| COC1═C(CC═C(C)C)C2═C(C(O)═C1)C(═O)C1═C(O2)C(O)═C(O)C═C1 | Dulxanthone A | FooDB |
| CC(CC(═O)C═C(C)C)C1CC(O)C(C)(O)C═C1 | Bisacurone B | FooDB |
| COC1═CC═C(OC\C═C/C#CC2═CC═C(O)C═C2)C═C1 | Asparenyol | FooDB |
| C\C(CC\C═C(/C)CCC(OC1OC(CO)C(O)C(O)C1O)C(C)(C)O)═C/CCC(C)(OC | Lyciumoside III | FooDB |
| 1OC(CO)C(O)C(O)C1O)C═C | | |
| CCCCCCCCCCCCCCCC\C═C\CC\C═C\CCCCCCCCCCC1═CC(C)OC1═O | Muridienin 3 | FooDB |
| CC(C)CCC1═CC2═C(N1)N═CC═C2 | 2-(3-Methylbutyl)- | FooDB |
| | 1H-pyrrolo[2,3- | |
| | b]pyridine | |
| COC1═C(O)C═C(C═C1O)[C@H]1OC2═C(C[C@H]1O)C(O)═CC(O[C@@H] | 4′-Methyl-(−)- | FooDB |
| 1O[C@@H]([C@@H](O)[C@H](O)[C@H]1O)C(O)═O)═C2 | epigallocatechin | |
| | 7-glucuronide | |
| COC1═CC2═C(C(═O)C═C(O2)C2═CC═C(O)C═C2)C(O)═C1C1OC(CO)C(O) | Spinosin B | FooDB |
| C(O)C1OC1OC(COC(═O)\C═C\C2═CC(OC)═C(O)C═C2)C(O)C(O)C1O | | |
| CN1CCCC1CC(═O)CC1CCC(CC(C)═O)N1C | Phygrine | FooDB |
| OCC1OC(OC2C(O)C(CO)OC(OCC3OC(O)C(O)C(O)C3O)C2O)C(O)C(O)C1O | Ata-D- | FooDB |
| | Galactopyranosyl- | |
| | (1->3)-beta-D- | |
| | galactopyranosyl- | |
| | (1->6)-D- | |
| | galactose | |
| CCCCC\C═C\CC(O)C(O)C(═O)CCCCCCC(O)═O | (9R,10S,12Z)- | FooDB |
| | 9,10-Dihydroxy- | |
| | 8-oxo-12- | |
| | octadecenoic | |
| | acid | |
| CCCCCCCCCCCCCCC1OC1CC\C═C/CCCCCCCCCCC1═CC(C)OC1═O | Sabadelin | FooDB |
| COC1═C(O)C═CC(═C1)C1COC2═CC(O)═CC═C2C1 | 4′,7-Dihydroxy-3′- | FooDB |
| | methoxyisoflavan | |
| COC1═CC2═C(C(═O)CC(O2)C2═CC(O)═C(OC)C═C2)C(OC2OC(CO)C(O)C(O)C2O)═C1 | Persiconin | FooDB |
| OCC1OC(OC2═CC(O)═CC(O)═C2C(═O)CCC2═CC═CC═C2)C(O)C(O)C1O | 2′,4′,6′- | FooDB |
| | Trihydroxydihydrochalcone | |
| | 2′- | |
| | glucoside | |
| COC1═CC(═CC(O)═C1OC)C1═CC(═O)C2═C(O1)C(OC)═C(OC)C═C2 | 3′-Hydroxy- | FooDB |
| | 4′,5′,7,8- | |
| | tetramethoxyflavone | |
| COC1═CC═C(C═C1)C1COC2═C(C═CC(O)═C2)C1═O | Dihydroformononetin | FooDB |
| OC1═CC(O)═C2C(═O)C(COC2═C1)C1═CC(O)═C(O)C═C1 | 3′,4′,5,7- | FooDB |
| | Tetrahydroxyisoflavanone | |
| CC(═O)C1CCS1 | xi-2- | FooDB |
| | Acetylthietane | |
| COC1═CC═C(C═C1)C1═C(OC)C(═O)C2═C(OC)C(OC)═C(Cl)C(OC)═C2O1 | 7-Chloro- | FooDB |
| | 3,4′,5,6,8- | |
| | pentamethoxyflavone | |
| CC(C1CC(OC(C)═O)C2(O)C3CC4OC44C(O)C═CC(═O)C4(C)C3CCC12C)C1 | Physapubenolide | FooDB |
| CC(C)═C(C)C(═O)O1 | | |
| CSC1═NC(CC(C)═O)C2═C(NC3═CC═CC═C23)S1 | 1-[4,9-Dihydro-2- | FooDB |
| | (methylthio)-1,3- | |
| | thiazino[6,5- | |
| | b]indol-4-yl]-2- | |
| | propanone | |
| CCC1C2N3CCC22C(CC1CC3)N(C(C)═O)C1═C2C═CC═C1OC | 14,19- | FooDB |
| | Dihydroaspidospermatine | |
| OC(CCC(O)═O)CC1═CC═C(O)C(O)═C1 | 4-Hydroxy-(3′,4′- | FooDB |
| | dihydroxyphenyl)- | |
| | valeric acid | |
| CC1C2C(CC3C4CC═C5CC(CCC5(C)C4CCC23C)OC2OC(CO)C(OC3OC(C) | Avenacoside A | FooDB |
| C(O)C(O)C3O)C(O)C2OC2OC(CO)C(O)C(O)C2O)OC11CCC(C)(COC2OC | | |
| (CO)C(O)C(O)C2O)O1 | | |
| COC1═CC(═CC(O)═C1OC)C(O)═O | 3,4-O- | FooDB |
| | Dimethylgallic | |
| | acid | |
| CC\C═C/C\C═C/C\C═C/CCCCCCCC(═O)OCC(O)COC1OC(COC2OC(CO)C | Gingerglycolipid A | FooDB |
| (O)C(O)C2O)C(O)C(O)C1O | | |
| C\C═C\S(═O)CC(NC(═O)CCC(N)C(O)═O)C(O)═O | gamma- | FooDB |
| | Glutamyl-S-(1- | |
| | propenyl)cysteine | |
| | sulfoxide | |
| CC1(C)CC(O)C2(C)CCC3(C)C(═CCC4C5(C)CCC(O)C(C)(C═O)C5CCC34C) | Sandosapogenol | FooDB |
| C2C1 | | |
| OC(C(O)═O)C(O)═O | Hydroxypropane | FooDB |
| | dioic acid | |
| CC1(C)CCCC2(C)C(\C═C\C(═C)C(O)CO)C(C)(O)C(O)C(O)C12 | Sterebin G | FooDB |
| COC1═CC(\C═C\C(═O)O[C@@]2(C[C@@H](O)[C@H](O)[C@@H](C2)OC | 1-Feruloyl-5- | FooDB |
| (═O)\C═C\C2═CC(O)═C(O)C═C2)C(O)═O)═CC═C1O | caffeoylquinic | |
| | acid | |
| [H][C@@]12O[C@]3([H])C═C(C)C(═O)C[C@]3(COC(C)═O)C(C)([C@H](OC | 8- | FooDB |
| (C)═O)[C@H]1O)C21CO1 | Oxodiacetoxyscirpenol | |
| CC(C)═C1CCC2(C)CCC═C(C)C2C1 | 3,7(11)- | FooDB |
| | Eudesmadiene | |
| COC1═C(\C═C/C(═O)C2═CC═C(O)C═C2)C═C(C(O)═C1)C(C)(C)C═C | Licochalcone A | FooDB |
| CCS(═O)CC(N)C(O)═O | Ethiin | FooDB |
| NC1═CC2═NC3═CC═CC═C3OC2═CC1═O | Questiomycin A | FooDB |
| CC(C)CCCC(C)CCCC(C)CCCC(C)(O)CCC1═CC(═O)C(C)═C(C)C1═O | gamma- | FooDB |
| | Tocopheryl | |
| | quinone | |
| ONC(═O)OCC1C2NC(═N)NC22N(CCC2(O)O)C(═N)N1O | N′- | FooDB |
| | Hydroxyneosaxitoxin | |
| COC1═CC═CC(OC)═C1C1═CC(═O)C2═C(O1)C═CC(OC)═C2OC | Zapotin | FooDB |
| COC1═CC(OC)═C2C(OC3═C2C(═O)OC2═C3C═CC(O)═C2)═C1 | Wairol | FooDB |
| CC(CCC1(O)OC2CC3C4CC(O)C5CC(OC6OC(CO)C(OC7OC(CO)C(O)C(O | Ampeloside Bf1 | FooDB |
| C8OC(CO)C(O)C(O)C8O)C7O)C(O)C6O)C(O)CC5(C)C4CCC3(C)C2C1C)C | | |
| OC1OC(CO)C(O)C(O)C1O | | |
| CC(═C)C1CCC2(C)CCC(═O)C(C)═C2C1 | (+)-4,11- | FooDB |
| | Eudesmadien-3- | |
| | one | |
| CCCCCC(═O)CCOC1OC(CO)C(O)C(O)C1O | 1-(beta-D- | FooDB |
| | Glucopyranosyloxy)- | |
| | 3-octanone | |
| COC1═CC═C(O)C2═C1CC(C(C)═C)C(C)(C2)C═C | Flavidulol B | FooDB |
| CC(═O)C1═C2OC3═C(C(O)═CC4═C3C═CC(C)(C)O4)C(═O)C2═CC2═C1C | Artonol B | FooDB |
| (═O)OC2(C)C | | |
| CCCCCCCCCCC1OC(CCC1O)C(O)CCC(O)C1CCC(CCCCCCCC(O)CC2═ | Mucocin | FooDB |
| CC(C)OC2═O)O1 | | |
| CC1OC(OCC2OC(OC3═C(OC4═CC(O)═CC5═[O+]C(C)═CC3═C45)C3═CC | Pyranodelphinin A | FooDB |
| (O)═C(O)C(O)═C3)C(O)C(O)C2O)C(O)C(O)C1O | | |
| CC(C)C1═C2C3═CC═C(CC(OC4OCC(O)C(O)C4O)C3(C)CCC2(C)CC1)C═O | Erinacine A | FooDB |
| OCC1OC(OC2═CC═C(\C═C\C(═O)OCC3CCN4CCCC34)C═C2)C(O)C(O)C1O | Thesinine 4′-O- | FooDB |
| | glucoside | |
| CCCCCC(O)CCCCC(O)C1CCC(O1)C1CCC(O1)C(O)CCCCCCCCCCC1═C | Atemoyacin B | FooDB |
| C(C)OC1═O | | |
| COC1═C(C═CC(O)═C1)C1COC2═C(C1)C═CC(O)═C2 | Isovestitol | FooDB |
| CC1C(C)C(═O)OC1C(O)C(C)(O)C1C(O)CC2C3CC4OC44C(O)C═CC(═O)C | Ixocarpalactone A | FooDB |
| 4(C)C3CCC12C | | |
| COC1═CC(═CC═C1O)[C@H]1COC2═CC(O)═CC═C2C1 | 3′-O-Methylequol | FooDB |
| O[C@H]1[C@H](OC2═C(O)C═C(\C═C\C(O)═O)C═C2)O[C@@H]([C@@H] | Caffeic acid 4-O- | FooDB |
| (O)[C@@H]1O)C(O)═O | glucuronide | |
| CC1OC(CC(O)C1O)OC1CCC2(C═O)C3CCC4(C)C(CCC4(O)C3CCC2(O)C1) | Corchoroside A | FooDB |
| C1═CC(═O)OC1 | | |
| CC1OC(OC2C(O)C(O)COC2OC(═O)C23CCC(C)(C)CC2C2═CCC4C5(C)CC | Medicagenic acid | FooDB |
| (O)C(O)C(C)(C5CCC4(C)C2(C)CC3)C(O)═O)C(O)C(O)C1OC1OCC(O)C(O) | 28-O-[b-D- | |
| C1O | xylosyl-(1->4)-a- | |
| | L-rhamnosyl-(1-> | |
| | 2)-a-L- | |
| | arabinosyl] ester | |
| OC1COC(OC2C3C4═C(O)C═C(O)C═C4OC2(OC2═CC(O)═C4CC(O)C(OC4═ | 3-O-alpha-L- | FooDB |
| C32)C2═CC═C(O)C(O)═C2)C2═CC═C(O)C(O)═C2)C(O)C1O | Arabinopyranosyl | |
| | proanthocyanidin | |
| | A5′ | |
| CC12CC(O)CC(C)(C1C(C(O)═O)C13CC(CCC21)C(═C)C3)C(O)═O | Gibberellin A110 | FooDB |
| O═C1C2C(COC3═CC4═C(OCO4)C═C23)OC2═C1C═C1C═COC1═C2 | Dolineone | FooDB |
| CC1OC(CC(O)C1OC1OC(CO)C(O)C(O)C1O)OC1CCC2(C)C(CCC3C2C(O) | Corchorusoside B | FooDB |
| CC2(C)C(CCC32O)C2═CC(═O)OC2)C1 | | |
| CCCCCCCCCCCCC(O)C1CCC(O1)C(O)CCCC(O)CCCCCCC(O)CC1═CC | Rolliacocin | FooDB |
| (C)OC1═O | | |
| OCC1OC(C(O)C(O)C1O)C1═C(O)C═C(O)C2═C1OC1═C(C═C(O)C(O)═C1) | Isomangiferin | FooDB |
| C2═O | | |
| COC1═C(O)C═C(C(OC2OC(CO)C(O)C(O)C2O)═C1)C1═COC2═CC(O)═C | Licoagroside A | FooDB |
| (OC)C═C2C1═O | | |
| CC(C)C(C)\C═C\C(C)C1CCC2C3═CC(═O)C4═CC(═O)CCC4(C)C3CCC12C | Ganodosterone | FooDB |
| CC1═C(O)C═C2C(═O)C(O)C3CCC(O)C(O)C3C2═C1 | Solanolone | FooDB |
| OCC1OC(C(O)C(O)C1O)C1═C(O)C(OC2═CC═C(C═C2)C2CC(═O)C3═C(O) | Occidentoside | FooDB |
| C═C(O)C═C3O2)═C(O)C(C(═O)\C═C\C2═CC═C(O)C═C2)═C1O | | |
| CC(C)CC(═O)O[C@@H]1C[C@@H]2CC[C@@]1(C)C2(C)C | Bornyl | FooDB |
| | isovalerate | |
| CCCCC1═NC2═CC═CC═C2N1 | 2-Butyl-1H- | FooDB |
| | benzimidazole | |
| CC(═O)OCC12CC(OC(═O)CC(C)(C)O)C(C)═CC1OC1C(O)C(OC(C)═O)C2 | 3′-Hydroxy-T2 | FooDB |
| (C)C11CO1 | Toxin | |
| COC1═C(OC2OC(C)C(O)C(O)C2O)C═C(O)C2═C1OC═C(C2═O)C1═CC═C | Isotectorigenin 4′- | FooDB |
| (OC2OC(CO)C(O)C(O)C2O)C═C1 | glucoside 7- | |
| | rhamnoside | |
| COC1═CC═C(\C═C\C2═CC(OC)═CC(═O)O2)C═C1 | Yangonin | FooDB |
| OCC1OC(OC2═CC(O)═C3C(═O)C(═COC3═C2)C2═CC═CC═C2O)C(O)C(O) | Isogenistein 7- | FooDB |
| C1O | glucoside | |
| CC(O)C(C)OC1OC(CO)C(O)C(O)C1O | 2,3-Butanediol | FooDB |
| | glucoside | |
| CC(C)CNC(═O)\C═C\CC\C═C\C1═CC2═C(OCO2)C═C1 | (E,E)-Futoamide | FooDB |
| CCCCCC1═NC(CCC)═CS1 | 2-Pentyl-4- | FooDB |
| | propylthiazole | |
| COC1═C(O)C═CC(═C1)C1OCC(CC2═CC3═C(OCO3)C═C2)C1CO | Sanshodiol | FooDB |
| CC1═C(C═C)C(═O)NC1CC1═C(C)C(CCC(O)═O)═C(N1)C1C(C(O)═O)C(═O) | Brassica napus | FooDB |
| C2═C1NC(CC1═C(CCO)C(C)═C(N1)C═O)═C2C | non-fluorescent | |
| | chlorophyll | |
| | catabolite 3 | |
| CC(C)(CS(O)(═O)═O)NC(═O)C═C | 2-Methyl-2-[(1- | FooDB |
| | oxo-2- | |
| | propenyl)amino]- | |
| | 1- | |
| | propanesulfonic | |
| | acid | |
| CC1═CC2═C(C(═O)C(C)(CO)C2)C(C)═C1CCCl | (S)-Pterosin K | FooDB |
| CC1(C)OC2═CC(═CC(O)═C2C═C1)C1═CC2═C(O1)C═C(O)C═C2 | Moracin D | FooDB |
| CC(C\C═C\C(C)(C)OC1OC(CO)C(O)C(O)C1O)C1CCC2(C)C3C═CC45OCC | Goyaglycoside e | FooDB |
| 3(CCC12C)C4CCC(OC1OC(CO)C(O)C(O)C1O)C5(C)C | | |
| COC1═C(O)C2═C(OC(═CC2═O)C2═CC(OC3OC(CO)C(O)C(O)C3O)═C(O) | 6-Methoxyluteolin | FooDB |
| C═C2)C═C1O | 3′-glucoside | |
| NCCCCNCCCNCCCCNCCCN | 1,17-Diamino- | FooDB |
| | 4,9,13- | |
| | triazaheptadecane | |
| CC1(C)CCC2(CCC3(C)C(═CCC4C5(C)CCC(OC6OCC(O)C(OC7OC(CO)C(O)C | Elatoside E | FooDB |
| (O)C7O)C6OC6OCC(O)C(O)C6O)C(C)(C)C5CCC34C)C2C1)C(O)═O | | |
| COC1C2═C(C(O)═C(O)C(═C2)C(C)C)C23CCCC(C)(C)C2C1(OC)OC3═O | 6,7-Dimethoxy-7- | FooDB |
| | epirosmanol | |
| CC(O)CC1═CC(═O)C2═C(O1)C═C(OC1OC(CO)C(O)C(O)C1O)C═C2C | Aloesol 7- | FooDB |
| | glucoside | |
| COC1═C(O)C═C2C(═O)OC3═C4C(═CC(OC5OC(CO)C(OC6OC(CO)C(O)C | 3-Methylellagic | FooDB |
| (O)C6O)C(O)C5O)═C3OC)C(═O)OC1═C24 | acid 2-(4- | |
| | galactosylglucoside) | |
| CC1(O)CCC(O)C2(C)CCC3C(OC(═O)C3═C)C12 | 1alpha- | FooDB |
| | Hydroxyarbusculin A | |
| CC1C2C(CC3C4CCC5CC(CCC5(C)C4CCC23C)OC2OC(COC3OC(C | Parillin | FooDB |
| O)C(O)C(O)C3O)C(OC3OC(C)C(O)C(O)C3O)C(O)C2OC2OC(CO)C(O)C(O)C2 | | |
| O)OC11CCC(C)CO1 | | |
| CC(C)═CCC\C(C)═C\CC\C(C)═C\CC\C(C)═C\CC\C(C)═C\CC\C(C)═C\CC\ | Arachisprenol 12 | FooDB |
| C(C)═C\CC\C(C)═C\CC\C(C)═C/CC\C(C)═C/CC\C(C)═C\CC\C(C)═C\CO | | |
| COC1═C(O)C═CC(CC(COC2OC(COC(═O)CC(C)(O)CC(O)═O)C(O)C(O)C | (8R,8′R)- | FooDB |
| 2O)C(COC2OC(COC(═O)CC(C)(O)CC(O)═O)C(O)C(O)C2O)CC2═CC(OC)═ | Secoisolariciresinol | |
| C(O)C═C2)═C1 | 9,9′-bis-[4- | |
| | carboxy-3- | |
| | hydroxy-3- | |
| | methylbutanoyl-(->6)- | |
| | glucoside] | |
| CC(C)(OC1OC(CO)C(O)C(O)C1O)C1CC2═C(O1)C═C1OC(═O)C═CC1═C2O | Celereoside | FooDB |
| COC1═CC2═C(C(O)═C1CC═C(C)C)C(═O)C1═C(O)C═CC(OC)═C1O2 | 1,8-Dihydroxy-3,5- | FooDB |
| | dimethoxy-2- | |
| | prenylxanthone | |
| CC(C)(OO)\C═C\CC(C)(OC1OC(CO)C(O)C(O)C1O)C1CCC2(C)C1C(O)CC | Notoginsenoside E | FooDB |
| 1C3(C)CCC(OC4OC(CO)C(O)C(O)C4OC4OC(CO)C(O)C(O)C4O)C(C)(C) | | |
| C3CCC21C | | |
| OCC1C(COC1C1═CC2═C(OCO2)C═C1)C(═O)C1═CC2═C(OCO2)C═C1 | 8′-Episesaminone | FooDB |
| CC1CC(C)(C)CC1═O | 2,4,4- | FooDB |
| | Trimethylcyclopentanone | |
| CCCCCCCCCCCCCCCC(═O)OC1═CC═C(CC═C)C═C1OC | Oryzarol | FooDB |
| CCCC1═CC(═O)OC2═C1C(O)═C(CC═C(C)C)C(O)═C2C(═O)CC(C)C | Mammea B/BA | FooDB |
| OCC1OC(C(O)C(O)C1O)C1═C(O)C(C2OCC(O)C(O)C2O)═C2OC(═CC(═O) | 6-beta-D- | FooDB |
| C2═C1O)C1═CC═C(O)C═C1 | Glucopyranosyl-8- | |
| | beta-D- | |
| | ribopyranosylapigenin | |
| [H][C@@]1(CC[C@@]2([H])[C@]3([H])CC[C@@]4([H])C[C@H](O)[C@H] | 6- | FooDB |
| (O)C[C@]4(C)[C@@]3([H])CC[C@]12C)[C@H](C)[C@@H](O)[C@H](O)[C | Deoxocastasterone | |
| @@H](C)C(C)C | | |
| CC(C)═CCC1CC23CC(CC═C(C)C)C(C)(C)C(CC═C(C)C)(C(═O)C(C(═O)C4═ | (+)- | FooDB |
| CC(O)═C(O)C═C4)═C2OC1(C)C)C3═O | Isoxanthochymol | |
| CCCCCC(O)CC(CCC1═CC(OC)═C(O)C═C1)OC(C)═O | [6]-Gingerdiol 3- | FooDB |
| | acetate | |
| CCCCC(O)CCCCCC(O)C1CCC(O1)C1CCC(O1)C(O)CCCCCCCCCCCCC | Asiminocin | FooDB |
| 1═CC(C)OC1═O | | |
| OC1═CC═CC2═C(O)C═CC(O)═C12 | 1,4,5- | FooDB |
| | Naphthalenetriol | |
| COC1═CC═CC2═C1NC1═C2C═C(CO)C═C1 | Mukoline | FooDB |
| COC(C1C(C(O)C(═O)N1C)C1═CC═CC═C1)C1═CC═CC═C1 | Lansimide 3 | FooDB |
| OC1═CC(O)═C2C(C(OC(═O)C3═CC(O)═C(O)C(O)═C3)C(OC2═C1)C1═CC═ | ent-Epicatechin- | FooDB |
| C(O)C(O)═C1)C1═C2OC(C(CC2═C(O)C═C1O)OC(═O)C1═CC(O)═C(O)C | (4alpha->8)-ent- | |
| (O)═C1)C1═CC═C(O)C(O)═C1 | epicatechin 3,3′- | |
| | digallate | |
| CN1C(CC(C)═O)C2═C(C═CC3═C2OCO3)C2═C1C1═CC3═C(OCO3)C═C1 | xi-8- | FooDB |
| C═C2 | Acetonyldihydrosanguinarine | |
| CC(═C)C1CC(═O)C2═C(C1)C(═O)C1═C(O2)C2═C(OC(C)(C)C═C2)C═C1O | Artonol A | FooDB |
| CC1═C(OC2OC(COC(═O)CC(C)(O)CC(O)═O)C(O)C(O)C2O)C(═O)C═CO1 | Licoagroside B | FooDB |
| OC1═CC(O)═C2CC(OC(═O)C3═CC(O)═C(O)C(O)═C3)C(OC2═C1)C1═CC | Theaflavin 3,3′- | FooDB |
| (O)═C(O)C2═C1C═C(C═C(O)C2═O)C1OC2═CC(O)═CC(O)═C2CC1OC(═O) | digallate | |
| C1═CC(O)═C(O)C(O)═C1 | | |
| CCC1═C(O)C(═N)CC1 | 2-Ethyl-5-imino-1- | FooDB |
| | cyclopenten-1-ol | |
| CC(C(═O)C1═C(O)C═C(O)C(O)═C1)C1═CC═C(O)C═C1 | 5′-Hydroxy-O- | FooDB |
| | desmethylangolensin | |
| COC1═CC(═CC(OC)═C1O)C12OC(C)(C(C1OC1OC(CO)C(O)C(O)C1O)C1═ | 5′- | FooDB |
| C(O)C═C(O)C═C1O2)C(C)═O | Methoxycastavinol | |
| CC1CCC2═C(C3═C(OC═C3C)C═C2C)C1═O | (S)-Curzeone | FooDB |
| COC1═CC═C(C═C1)C1═CC(═O)C2═C(OC)C═C(OC)C═C2O1 | 4′,5,7- | FooDB |
| | Trimethoxyflavone | |
| CC(C)(OC1OC(CO)C(O)C(O)C1O)C1═CC2═C(O1)C═CC1═C2OC(═O)C═C1 | Edulisin VI | FooDB |
| CCCCCCCCCCC(O)C1CCC(O1)C(O)CCC(O)C1CCC(CCCCCCCCCC2═C | Squamostatin E | FooDB |
| C(C)OC2═O)O1 | | |
| CC1C2CCC(C)CN2C2CC3C4CC═C5CC(CCC5(C)C4CCC3(C)C12)OC1O | gamma- | FooDB |
| C(CO)C(O)C(O)C1O | Chaconine | |
| CC1(C)C2CCC3(C)C(CC═C4C5CC(C)(CCC5(C)CCC34C)C(O)═O)C2(C)C | Katononic acid | FooDB |
| CC1═O | | |
| COC1═CC2═C(C(═O)C═C(O2)C2═CC═C(O)C═C2)C(O)═C1C1OC(CO)C(O) | Spinosin C | FooDB |
| C(O)C1OC1OC(COC(═O)\C═C\C2═CC═C(O)C═C2)C(O)C(O)C1O | | |
| CC1(C)CC(O)C2(CO)C(O)CC3(C)C(═CCC4C5(C)CCC(O)C(C)(C═O)C5CC | Camelliagenin B | FooDB |
| C34C)C2C1 | | |
| COC1═CC2═C(C3C═COC3O2)C2═C1C(═O)C1═C(O2)C(OC)═C(OC)C═C1O | 5,6- | FooDB |
| | Dimethoxysterigmatocystin | |
| COC1═C(O)C═CC(═C1)C1═C(OC2OC(COC3OC(C(C)O)C(O)C3O)C(O)C | Isorhamnetin 3-O- | FooDB |
| (O)C2O)C(═O)C2═C(O)C═C(O)C═C2O1 | [b-L- | |
| | rhamnofuranosyl- | |
| | (1->6)-D- | |
| | glucopyranoside] | |
| NCCCC(N)C(═O)NCCS(O)(═O)═O | N-Ornithyl-L- | FooDB |
| | taurine | |
| O═C(CC1═CC═CC═C1)OC\C═C/C1═CC═CC═C1 | Cinnamyl | FooDB |
| | phenylacetate | |
| OC1═CC2═C(C[C@@H](OC(═O)C3═CC(O)═C(O)C(O)═C3)[C@H](O2)C2═ | (−)- | FooDB |
| CC(OC(═O)C3═CC(O)═C(O)C(O)═C3)═C(O)C(O)═C2)C(O)═C1 | Epigallocatechin | |
| | 3,3′-di-gallate | |
| COC1═C(O)C═C(C═C1)C1═COC2═CC(O)═CC(O)═C2C1═O | Pratensein | FooDB |
| COC1═CC2═C(OCC(C2OS(O)(═O)═O)C2═C(O)C(OC)═CC(\C═C\C(O)═O)═ | Torvanol A | FooDB |
| C2)C═C1 | | |
| COC1═C(O)C═CC(\C═C/C(═O)OC2C(OC3═CC(O)═C4C(OC(C5═CC═C(O | Kaempferol 3,4′- | FooDB |
| C6OC(CO)C(O)C(O)C6O)C═C5)═C(OC5OC(CO)C(O)C(O)C5O)C4═O)═C3) | diglucoside 7-(2- | |
| OC(CO)C(O)C2O)═C1 | feruloylglucoside) | |
| OCC1OC(OC2C(O)C(O)C(CO)OC2OC2═C(OC3═CC(OC4OC(CO)C(O)C | Quercetin 3-(2″- | FooDB |
| (O)C4O)═CC(O)═C3C2═O)C2═CC(O)═C(O)C═C2)C(O)C(O)C1O | glucosylgalactoside) | |
| | 7-glucoside | |
| CC(═C)[C@H]1CC[C@@](C)(O)CC1 | trans-beta- | FooDB |
| | Terpineol | |
| COC1═CC(\C═C/C(═O)OC2OC(COC3OC(CO)C(O)C(O)C3O)C(OC(═O)\C═ | Raphanusol A | FooDB |
| C/C3═CC(OC)═C(O)C(OC)═C3)C(O)C2O)═CC(OC)═C1O | | |
| CC(C)C1═CC2═C(C═C1)C1(C)CCCC(═C)C1CC2 | 18-Nor- | FooDB |
| | 4(19),8,11,13- | |
| | abietatetraene | |
| CC1CCC(═O)C2CC3(O)OC(═O)C(C)═C3CC12C | Istanbulin A | FooDB |
| CC(Cl)C(C)(O)C(═O)OC1C2═C(C)C(═O)OC2(O)CC2CCC(O)C(C)C12C | 3b,8b-Dihydroxy- | FooDB |
| | 6b-(3-chloro-2- | |
| | hydroxy-2- | |
| | methylbutanoyloxy)- | |
| | 7(11)- | |
| | eremophilen-12,8- | |
| | olide | |
| COC1═C(O)C═C(C═C1O[C@@H]1O[C@@H]([C@@H](O)[C@H](O)[C@ | 4′-Methyl-(−)- | FooDB |
| H]1O)C(O)═O)[C@H]1OC2═C(C[C@H]1O)C(O)═CC(O)═C2 | epigallocatechin | |
| | 3′-glucuronide | |
| OCC1OC(OC2C(O)C(O)C(COC(═O)\C═C\C3═CC═C(OC4OC(CO)C(O)C(O) | Cyanidin 3-[6-(4- | FooDB |
| C4O)C═C3)OC2OC2═C([O+]═C3C═C(O)C═C(OC4OC(CO)C(O)C(O)C4O) | glucosylcoumaryl) | |
| C3═C2)C2═CC(O)═C(O)C═C2)C(O)C(O)C1O | sophoroside] 5- | |
| | glucoside | |
| CC(CC(═O)CC(C)C(O)═O)C1CC(O)C2(C)C3═C(C(═O)CC12C)C1(C)CCC | Ganolucidic acid C | FooDB |
| (O)C(C)(CO)C1CC3 | | |
| CC(C)═CCC1═C(O)C═CC(═C1)C12OC34C(OC5═C3C═CC(O)═C5CC═C(C) | Licoagrodin | FooDB |
| C)OC3═CC(O)═CC═C3C4C1C(═O)C1═CC(CC═C(C)C)═C(O)C═C1O2 | | |
| CCCCCCCCCCCCCCCCCCCC(═O)CC(═O)CCCCCCCCCC | 11,13- | FooDB |
| | Dotriacontanedione | |
| OC(═O)CC(NC(═O)C1═C(O)C═CC═C1)C(O)═O | N- | FooDB |
| | Salicyloylaspartic | |
| | acid | |
| CC1═CC(O)═CC(O)═C1C(═O)OC1CC2(C)C3C(CC(C)(C)C3O)C═C(C═O)C | 10- | FooDB |
| 12O | Hydroxymelleolide | |
| CCCCC\C═C/C\C═C/CCCCCCCC(═O)CC(O)COC(C)═O | Persin | FooDB |
| CCC1NC(═O)C2C(Cl)C(Cl)CN2C(═O)C(CO)NC(═O)CC(NC(═O)C(CO)NC1═ | Cyclochlorotine | FooDB |
| O)C1═CC═CC═C1 | | |
| CC(C)[C@@H]1CC[C@H](C)C[C@@H]1O | (−)-Neoisomenthol | FooDB |
| [H][C@@]1(CC[C@@]2(C)C3═C(CC[C@]12C)[C@@]1(C)CC[C@H](O)C | Trametenolic acid B | FooDB |
| (C)(C)[C@]1([H])CC3)[C@@H](CCC═C(C)C)C(O)═O | | |
| COC1═C(O)C═C(C═C1O)C1═[O+]C2═CC(O)═CC(O)═C2C═C1O[C@@H]1 | 4′-O- | FooDB |
| O[C@H](CO[C@@H]2O[C@@H](C)[C@H](O)[C@@H](O)[C@H]2O)[C@ | Methyldelphinidin | |
| @H](O)[C@H](O)[C@H]1O | 3-O-rutinoside | |
| CC(C)(O)C\C(SC1OC(CO)C(O)C(O)C1O)═N/OS(O)(═O)═O | Glucoconringiin | FooDB |
| COC(═O)CC(═O)OCC1OC(OC2═CC(O)═C3C(OC═C(C3═O)C3═CC═C(OC) | Biochanin A 7-(6- | FooDB |
| C═C3)═C2)C(O)C(O)C1O | methylmalonylglucoside) | |
| O═C(OC1CCCCC1)\C═C/C1═CC═CC═C1 | Cyclohexyl | FooDB |
| | cinnamate | |
| OC1═CC═CC(C[C@H]2COC(═O)[C@@H]2CC2═CC(O)═C(O)C═C2)═C1 | 4- | FooDB |
| | Hydroxyenterolactone | |
| CC1═C(CO)C(═O)OC(C1)C(C)(O)C1CCC2C3CC═C4CC(CC(═O)C4(C)C3 | (3b,20R,22R)- | FooDB |
| CCC12C)OC1OC(CO)C(O)C(O)C1O | 3,20,27- | |
| | Trihydroxy-1- | |
| | oxowitha-5,24- | |
| | dienolide 3- | |
| | glucoside | |
| COC1═CC(O)═C2C(═O)CC(OC2═C1)C1═CC(O)═C(OC)C═C1 | 3′,5-Dihydroxy- | FooDB |
| | 4′,7- | |
| | dimethoxyflavanone | |
| CC1C2C(CC3C4CCC5CC(CCC5(C)C4CC(═O)C23C)OC2OC(CO)C(OC3O | Agavoside F | FooDB |
| C(CO)C(OC4OC(CO)C(O)C(OC5OC(C)C(OC6OC(C)C(O)C(O)C6O)C(O)C | | |
| 5OC5OC(C)C(O)C(O)C5O)C4OC4OCC(O)C(O)C4O)C(O)C3O)C(O)C2O) | | |
| OC11CCC(C)CO1 | | |
| CC1(C)CCC2(CCC3(C)C(═CCC4C5(C)CCC(OC6OCC(O)C(O)C6O)C(C)(C) | 28- | FooDB |
| C5CCC34C)C2C1)C(═O)OC1OC(CO)C(O)C(O)C1O | Glucosyloleanolic | |
| | acid 3-arabinoside | |
| CC12OC3═C(C═CC(O)═C3)C(C1O)C1═C(OC3═C(C(O)═CC(═C3)C3═CC4═ | Mulberrofuran S | FooDB |
| C(O3)C═C(O)C═C4)C1═C2)C1═C(O)C═C(O)C═C1 | | |
| CC(═C)C1CCC2(CCC3(C)C(CCC4C5(C)CC(O)C(OC(═O)\C═C\C6═CC(O)═ | 2alpha- | FooDB |
| C(O)C═C6)C(C)(C)C5CCC34C)C12)C(O)═O | Hydroxypyracrenic | |
| | acid | |
| OC1═CC═C(\C═C\C(═O)C2═CC═C(O)C═C2O)C═C1 | Isoliquiritigenin | FooDB |
| COC1═CC(O)═C(C(═O)OC2CC3(C)C4C(CC(C)(C)C4O)C═C(C═O)C23O)C(C)═C1 | Melleolide H | FooDB |
| CC(═O)CCC1═CC═C(OC2OC(COC(═O)C3═CC(O)═C(O)C(O)═C3)C(O)C(O)C2O)C═C1 | Lindleyin | FooDB |
| CC1(C)OC2(C)CC(O)C1CC2OC1OC(CO)C(O)C(O)C1O | Foeniculoside IX | FooDB |
| CCCCCCC\C═C\CCC═C | (Z)-1,5- | FooDB |
| | Tridecadiene | |
| CC(C)CC(O)C(═O)NC1C(OC2═CC═C(CC(NC(═O)C(CC3═CC═CC═C3)NC | Vignatic acid A | FooDB |
| 1═O)C(O)═O)C═C2)C(C)C | | |
| COC1═CC(OC2OC(COC3OC(CO)C(O)C(OC4OC(COC5OC(CO)C(O)C(O) | Cassiaside B2 | FooDB |
| C5O)C(O)C(O)C4O)C3O)C(O)C(O)C2O)═C2C(O)═C3C(═O)C═C(C)OC3═ | | |
| CC2═C1 | | |
| CC(═C)[C@H]1CC[C@](C)(O)CC1 | cis-beta-Terpineol | FooDB |
| COC1═C(O)C2═C(OC(═CC2═O)C2═CC(O)═C(OC3OC(CO)C(O)C(O)C3O) | Eupafolin 4′- | FooDB |
| C═C2)C═C1O | glucoside | |
| COC1═C(OC2OC(CO)C(O)C(O)C2O)C═CC(\C═C\C(═O)OCC2OC(OC3═C | Cyanidin 3-[6-(4- | FooDB |
| ([O+]═C4C═C(O)C═C(OC5OC(CO)C(O)C(O)C5O)C4═C3)C3═CC(O)═C(O) | glucosylferuloyl)sophoroside] | |
| C═C3)C(OC3OC(CO)C(O)C(O)C3O)C(O)C2O)═C1 | 5- | |
| | glucoside | |
| CC(C)CC(═O)C1═C(O)C2═C(OC3C4C(CC\C(C)═C/CCC3(C)C2)C4(C)C)C | Euglobal VII | FooDB |
| (C═O)═C1O | | |
| COC1═C(O)C═C(C═C1O)[C@H]1OC2═CC(O)═CC(O)═C2C[C@H]1OC(═O) | 4′-Methyl-(−)- | FooDB |
| C1═CC(O)═C(OC)C(O)═C1 | epigallocatechin | |
| | 3-(4-methyl- | |
| | gallate) | |
| CC1CCC2(CCC3(C)C(═CCC4C5(C)CC(O)C(O)C(C)(COC(═O)\C═C\C6═C | 23-trans-p- | FooDB |
| C═C(O)C═C6)C5CCC34C)C2C1(C)O)C(O)═O | Coumaroyloxytormentic | |
| | acid | |
| O═C1CC2CCCC(C1)N2 | 9- | FooDB |
| | Azabicyclo[3.3.1]nonan- | |
| | 3-one | |
| CC(CCC═C(C)C)C1C(CC2(C)C3C(O)C═C4C(CCC(OC5OCC(OC(C)═O)C | Hebevinoside XIII | FooDB |
| (OC(C)═O)C5O)C4(C)C)C3(C)CCC12C)OC1OC(COC(C)═O)C(OC(C)═O)C | | |
| (O)C1O | | |
| COC1═CC(CC═C)═CC(OC)═C1OC(C)C(O)C1═CC(OC)═C(OC)C(OC)═C1 | 2-(4-Allyl-2,6- | FooDB |
| | dimethoxyphenoxy)- | |
| | 1-(3,4,5- | |
| | trimethoxyphenyl)- | |
| | 1-propanol | |
| COC1═CC(O)═CC2═C1C(═O)C1═C(C═C(C)C(O)═C1)C2═O | Cajaquinone | FooDB |
| CC(═O)C1═C(O)C═CC(O)═C1 | 2′,5′- | FooDB |
| | Dihydroxyacetophenone | |
| OC1C(O)C2OC(═O)C3═CC(O)═C(O)C(O)═C3C3═C(O)C(O)═C(O)C═C3C | Guavin B | FooDB |
| (═O)OCC2OC1OC1═CC(O)═C(C(═O)C2═CC═CC═C2)C(O)═C1 | | |
| COC(═O)C1═COC(OC2OC(CO)C(O)C(O)C2O)C(C═C)C1CC(═O)OCCC1═ | Oleuroside | FooDB |
| CC(O)═C(O)C═C1 | | |
| COC1═CC2═C(C═C1)C═C(C(═O)O2)C(C)(C)C═C | 3-(1,1- | FooDB |
| | Dimethylallyl)herniarin | |
| CC(C)═CCC1═C(O)C2═C(OC(═CC2═O)C2═C(O)C═C(O)C═C2)C═C1O | Artocarpesin | FooDB |
| CC1(C)CCC2(CCC3(C)C(═CCC4C5(C)CCC(OC6OCC(O)C(O)C6OC6OC | Medicoside I | FooDB |
| (CO)C(O)C(O)C6OC6OCC(O)C(O)C6O)C(C)(CO)C5CCC34C)C2C1)C(═O) | | |
| OC1OC(CO)C(O)C(O)C1O | | |
| CC(═O)OC1(CO)COC(OC2C(O)C(O)C(CO)OC2OC2═CC═C(C═C2)C2CC | Liquiritigenin 4′-[3- | FooDB |
| (═O)C3═C(O2)C═C(O)C═C3)C1O | acetylapiosyl-(1-> | |
| | 2)-glucoside] | |
| CC(C)CCCC(C)CCCC(C)CCC\C(C)═C\CC\C(C)═C\CO | (2Z,6E)- | FooDB |
| | 3,7,11,15,19- | |
| | Pentamethyl-2,6- | |
| | eicosadien-1-ol | |
| CC1(C)CC(O)C2(CO)C(O)CC3(C)C(═CCC4C5(C)CCC(O)C(C)(CO)C5CC | Camelliagenin C | FooDB |
| C34C)C2C1 | | |
| OC[C@H]1O[C@@H](OC2═CC3═C(C═C2)C(═O)C(CO3)C2═CC═C(O)C═ | Dihydrodaidzin | FooDB |
| C2)[C@H](O)[C@@H](O)[C@@H]1O | | |
| COC1═CC═C(\C═C\C2═CC(O)═C(OC)C(CO)═C2)C═C1O | 3′-Hydroxy- | FooDB |
| | 3,4,5,4′- | |
| | tetramethoxystilbene | |
| OCC1OC(OC2═CC═C(\C═C\C(O)═O)C═C2)C(OC2OCC(O)(CO)C2O)C(O) | cis-p-Coumaric | FooDB |
| C1O | acid 4-[apiosyl-(1-> | |
| | 2)-glucoside] | |
| CCC(\C═C\C(C)C1CCC2C3CC═C4CC(O)CCC4(C)C3CCC12C)C(C)═C | 22- | FooDB |
| | Dehydroclerosterol | |
| CC(═C)C1CCC2(CCC3(C)C(CCC4C5(C)CC(OC(═O)C6═CC(O)═C(O)C═C | 2-O- | FooDB |
| 6)C(O)C(C)(C)C5CCC34C)C12)C(O)═O | Protocatechuoylalphitolic | |
| | acid | |
| CC(C)C(NC(═O)CCC(N)C(O)═O)C(O)═O | L-gamma- | FooDB |
| | glutamyl-L-valine | |
| CC1(C)CCC2(CCC3(C)C(═CCC4C5(C)CCC(OC6OC(C(OC7OC(CO)C(O) | Tuberoside A | FooDB |
| C(O)C7O)C(O)C6O)C(O)═O)C(C)(C)C5CCC34C)C2C1)C(═O)OC1OC(CO) | (Ullucus | |
| C(O)C(O)C1O | tuberosus) | |
| CCO\C(S)═N\CC1═CC═C(O[C@@H]2O[C@@H](C)[C@H](OC(C)═O)[C@ | N-[(4- | FooDB |
| @H](OC(C)═O)[C@H]2OC(C)═O)C═C1 | hydroxyphenyl)methyl] | |
| | ethoxycarbothioamide | |
| | 4′-(tri- | |
| | acetylrhamnoside) | |
| CC(C)═CCC1═C(O)C═CC(C(═O)\C═C\C2═C(O)C═C(O)C═C2)═C1O | Morachalcone A | FooDB |
| OCC1OC(OC2═CC3═C(O)C═C(O)C═C3[O+]═C2C2═CC═C(O)C═C2)C(O) | Pelargonidin 3- | FooDB |
| C(O)C1O | galactoside | |
| COC1═CC(OC)═C(C(C)═O)C(O)═C1C | 2′-Hydroxy-4′,6′- | FooDB |
| | dimethoxy-3′- | |
| | methylacetophenone | |
| CC1C2C(CC3C4CCC5CC(CCC5(C)C4CC(═O)C23C)OC2OC(CO)C(O)C(O | Schidigerasaponin | FooDB |
| C3OCC(O)C(O)C3O)C2OC2OC(CO)C(O)C(O)C2O)OC11CCC(═C)CO1 | B1 | |
| O═C1C2═CC═CC═C2C2═NC═CC3═C2C1═NC═C3 | Sampangine | FooDB |
| CC(C)CCC(O)C(C)C1C(CC2C3CC═C4CC(O)CC(OC5OC(C)C(OC6OC(CO) | Alliosterol 1-(4″- | FooDB |
| C(O)C(O)C6O)C(O)C5O)C4(C)C3CCC12C)OC1OC(CO)C(O)C(O)C1O | galactosylrhamnoside) | |
| | 16- | |
| | galactoside | |
| OCC1OC(OC2C(OC3═C([O+]═C4C═C(O)C═C(OC5OC(CO)C(O)C(O)C5O) | Delphinidin 3- | FooDB |
| C4═C3)C3═CC(O)═C(O)C(O)═C3)OC(CO)C(O)C2O)C(O)C(O)C1O | sophoroside 5- | |
| | glucoside | |
| CCCCC\C═C\CC1CC(═O)OC1═O | Dihydro-3-(2- | FooDB |
| | octenyl)-2,5- | |
| | furandione | |
| OCC1OC(OC2═CC(O)═CC3═[O+]C(═C(OC4OC(COC(═O)\C═C\C5═CC(O)═ | YGM 1B | FooDB |
| C(O)C═C5)C(O)C(O)C4OC4OC(COC(═O)\C═C\C5═CC(O)═C(O)C═C5)C | | |
| (O)C(O)C4O)C═C23)C2═CC(O)═C(O)C═C2)C(O)C(O)C1O | | |
| CC1═CN2C═CC═C2N═N1 | 3- | FooDB |
| | Methylpyrrolo[1,2- | |
| | a]pyrazine | |
| COC1═CC(OC2OC(CO)C(O)C(O)C2O)═C2C(OC)═C(C(C)═O)C(OC3OCC | Cassitoroside | FooDB |
| (O)(CO)C3O)═CC2═C1 | | |
| OC1═CC═C(\C═C/C\C═C/C2═CC(O)═CC═C2)C═C1 | (1Z,4Z)-1,5-bis(4- | FooDB |
| | hydroxyphenyl)- | |
| | 1,4-pentadiene | |
| CC1OC(OC2C(O)C(O)C(CO)OC2OC2CCC3(C)C(CCC4(C)C3CCC3C5C6 | Saponin D | FooDB |
| (CC43CO6)OC(CC5(C)OC3OC(C)C(O)C(O)C3O)C═C(C)C)C2(C)C)C(O)C | | |
| (O)C1O | | |
| [H][C@]12C[C@@]3([H])[C@]4([H])CC[C@]5([H])C[C@@H](O)CC[C@]5 | Smilagenin | FooDB |
| (C)[C@@]4([H])CC[C@]3(C)[C@@]1([H])[C@H](C)[C@@]1(CC[C@@H](C) | | |
| CO1)O2 | | |
| CC(C)═CCC\C(C)═C/COC═O | Neryl formate | FooDB |
| C\C(\C═C/C═C(/C)\C═C\C1═C(C)C(O)C(O)CC1(C)C)═C/C═C/C═C(/C)\C═C/ | Idoxanthin | FooDB |
| C═C(/C)\C═C\C1═C(C)C(═O)C(O)CC1(C)C | | |
| CC1(C)OC2═CC3═C(C(O)═C2C═C1)C(═O)C═C(O3)C1═C(O)C═C(O)C═C1 | Cycloartocarpesin | FooDB |
| COC1═CC(O)═C(C═C1)C1═COC2═C(C(O)═CC(O)═C2OC)C1═O | 5,7-Dihydroxy- | FooDB |
| | 8,4′- | |
| | dimethoxyisoflavone | |
| COC1═CC(═O)OC1(O)C(═O)CCCCCCC\C═C\C\C═C\CC═C | 5-Hydroxy-4- | FooDB |
| | methoxy-5-(1-oxo- | |
| | 9,12,15- | |
| | hexadecatrienyl)- | |
| | 2(5H)-furanone | |
| CC1═CC2═C(C═C1O)C1(C)C(O)CC(O)(O2)C1(C)C | Enokipodin C | FooDB |
| OC1═CC═C2C(═O)C(COC2═C1)C1═CC═C(O)C(O)═C1 | 3′- | FooDB |
| | Hydroxydihydrodaidzein | |
| COC1═CC(═CC(OC)═C1O)C(O)C(C)OC1═C(OC)C═C(CC═C)C═C1OC | 2-(4-Allyl-2,6- | FooDB |
| | dimethoxyphenoxy)- | |
| | 1-(3-hydroxy- | |
| | 4,5- | |
| | dimethoxyphenyl)- | |
| | 1-propanol | |
| COC1═C(OC2OC(COC3OC(CO)C(O)C3O)C(O)C(O)C2O)C2═CC(═C1OC) | Myricanene B 5- | FooDB |
| C1═C(O)C═CC(C\C═C\CCCC2)═C1 | [arabinosyl-(1->6)- | |
| | glucoside] | |
| OC1═CC═C(C═C1)C1═COC2═CC(O)═C(O)C(O)═C2C1═O | 5,6,7,4′- | FooDB |
| | Tetrahydroxyisoflavone | |
| OC(C(O)═O)C(O)(CC1═CC═C(O)C═C1)C(O)═O | (2R,3S)-Piscidic | FooDB |
| | acid | |
| COC1═CC2═C(OCO2)C═C1C1═COC2═CC(OC3OC(CO)C(O)C(O)C3OC(═ | 7-Hydroxy-2′,5,6- | FooDB |
| O)\C═C/C3═CC═C(O)C═C3)═C(OC)C(OC)═C2C1═O | trimethoxy-4′,5′- | |
| | methylenedioxyisoflavone | |
| | 7-(2-p- | |
| | coumaroylglucoside) | |
| COC1═CC2═C(C(═O)C═C(O2)C2═CC═CC═C2)C(OC)═C1C | Dimethylstrobochrysin | FooDB |
| COC1═C(O)C(O)═CC(═C1)C1═[O+]C2═C(C═C1O[C@@H]1O[C@H](CO[C | Petunidin 3- | FooDB |
| @@H]3O[C@@H](C)[C@H](O)[C@@H](O)[C@H]3O)[C@@H](O)[C@H] | rutinoside | |
| (O)[C@H]1O)C(O)═CC(O)═C2 | | |
| CC1OC(OCC2OC(OC3CCC4(C)C(CCC5(C)C4CCC4C6C(C)(O)C(CC═C(C) | Mabioside D | FooDB |
| C)OC6(O)C(O)C54C)C3(C)C)C(O)C(O)C2O)C(O)C(O)C1O | | |
| COC1═CC2═C(OC(═O)C(═C2)C(C)(C)C═C)C═C1O | 3-(1,1- | FooDB |
| | Dimethylallyl)scopoletin | |
| COC1═CC(O)═C2C(═O)CC(OC2═C1)C1═CC(OC2OC(CO)C(O)C(O)C2O)═ | Persicogenin 3′- | FooDB |
| C(OC)C═C1 | glucoside | |
| C\C(CC\C═C(/C)C(O)═O)═C/CC\C(C)═C\CCC(C)(OC1OC(CO)C(O)C(O)C1 | Capsianoside IV | FooDB |
| OC1OC(CO)C(O)C(O)C1O)C═C | | |
| COC(C)(C)C1═CC2═C(O1)C═CC1═C2OC(═O)C═C1 | Archangin | FooDB |
| OCC(O)C(O)(CO)CO | L-2- | FooDB |
| | (Hydroxymethyl)- | |
| | 1,2,3,4- | |
| | butanetetrol | |
| OCC1OC(O)C(OC(═O)\C═C\C2═CC═C(O)C═C2)C(O)C1O | 2-O-p-Coumaroyl- | FooDB |
| | D-glucose | |
| OC(═O)C1═CC2═C(OCO2)C═C1 | 3,4- | FooDB |
| | Methylenedioxybenzoic | |
| | acid | |
| COC1═C(O)C2═C(C═C1)C(═O)C1═C(N2C)C2═C(OC(C)(C)C═C2)C═C1O | 2′,2′- | FooDB |
| | Dimethyl(pyrano- | |
| | 5′,6′:3:4)-1,5- | |
| | dihydroxy-6- | |
| | methoxy-10- | |
| | methylacridone | |
| CC(C)═CCC\C(C)═C\C═C\C(\C)═C\C═C\C(\C)═C\C═C\C═C(/C)\C═C\C═C(/ | (all-E)-6′-Apo-y- | FooDB |
| C)\C═C\C═O | caroten-6′-al | |
| CC1C2C3CCC4C5(C)CCC(OC(C)═O)C(C)(C)C5CCC4(C)C3(C)CCC2(C)C | psi-Taraxasteryl | FooDB |
| C═C1C | acetate | |
| OCC(CC1═CC(O)═C(O)C═C1)C(CO)CC1═CC═CC(O)═C1 | 4- | FooDB |
| | Hydroxyenterodiol | |
| CC1C(C)C2(CCCO2)OC2C1OC1CC3OC4CC5OC6C\C═C/CC7OC8C═CC | Pacific Ciguatoxin | FooDB |
| 9OC%10C(CC9OC8C═CC7OC6CC(O)C5(C)OC4CC(C)CC3OC1C(C)C2O) | 4A | |
| OC1CC═CC(OC1C%10O)\C═C/C═C | | |
| COC1═C(OC2OC(C(O)C(O)C2O)C(O)═O)C═CC(═C1)C1═C(OC)C(═O)C2═ | 4′,5-Dihydroxy- | FooDB |
| C(O)C3═C(OCO3)C═C2O1 | 3,3′-dimethoxy- | |
| | 6,7- | |
| | methylenedioxyflavone | |
| | 4′- | |
| | glucuronide | |
| CC1C═C2C(COC2═O)C(O)C2CC(C)(C)CC12O | Blennin D | FooDB |
| CC1(C)CC(═O)C2═C(O1)C═C(OC1OC(CO)C(O)C(O)C1O)C(CCC(O)═O)═ | 6-(2- | FooDB |
| C2 | Carboxyethyl)-7- | |
| | hydroxy-2,2- | |
| | dimethyl-4- | |
| | chromanone | |
| | glucoside | |
| CCCCCCCCCCCCCC1CC2═C(C(═O)O1)C(O)═CC═C2 | (R)-3,4-Dihydro-8- | FooDB |
| | hydroxy-3-tridecyl- | |
| | 1H-2-benzopyran- | |
| | 1-one | |
| CC(C)═CCC1═CC(═CC═C1O)C1CC(═O)C2═C(O1)C(CC═C(C)C)═C(O)C═C2 | (S)-4′,7- | FooDB |
| | Dihydroxy-3′,8- | |
| | diprenylflavanone | |
| OC1C(COC(═O)\C═C\C2═CC═CC═C2)OC(OC(═O)C2═CC(O)═C(O)C(O)═ | 6-Cinnamoyl-1,2- | FooDB |
| C2)C(OC(═O)C2═CC(O)═C(O)C(O)═C2)C1O | digalloylglucose | |
| CC(O)CC(═O)CC1═CC(OC2OC(CO)C(O)C(O)C2O)═CC2═C1C(═O)C═C(C) | 7-Hydroxy-5-(4- | FooDB |
| O2 | hydroxy-2- | |
| | oxopentyl)-2- | |
| | methylchromone | |
| | 7-glucoside | |
| CCCCCCCOC(═O)\C═C/C1═CC═CC═C1 | Heptyl cinnamate | FooDB |
| COC1═CC2═C(C═C1)C(═O)C(═C(C)O2)C1═CC═CC═C1 | 7-Methoxy-2- | FooDB |
| | methylisoflavone | |
| COC1═CC2═C(OC3═C2C(C)═CC(OC)═C3O)C(═C1)C(O)═O | Porric acid A | FooDB |
| CC(C)═CCC\C(C)═C\CC1═C(O)C2═C(OC3═C(C═CC═C3O)C2═O)C═C1O | Mangostinone | FooDB |
| CC1OC(OC2C(O)C(OC(OC3CCC4(C)C(CCC5(C)C4CC═C4C6CC(C)(C)C | Quillaic acid 3- | FooDB |
| CC6(C(O)CC54C)C(═O)OC4OC(C)C(OC(C)═O)C(OC5OC(CO)C(O)C(O)C | [galactosyl-(1->2)- | |
| 5O)C4OC4OC(C)C(OC5OCC(O)C(O)C5O)C(O)C4O)C3(C)C═O)C2OC2O | [rhamnosyl-(1-> | |
| C(CO)C(O)C(O)C2O)C(O)═O)C(O)C(O)C1O | 3)]-glucuronide]28- | |
| | [glucosyl-(1-> | |
| | 3)-[xylosyl-(1-> | |
| | 4)-rhamnosyl-(1-> | |
| | 2)]-4-acetyl- | |
| | fucosyl] ester | |
| CC(C1CC(C)═C(C)C(═O)O1)C12OC1C(OC(C)═O)C1(O)C3CC4OC44C(O) | Physagulin C | FooDB |
| C═CC(═O)C4(C)C3CCC21C | | |
| NC1═CC═CC═C1C(═O)OC1═CC2═CC═CC═C2C═C1 | 2-Naphthalenol 2- | FooDB |
| | aminobenzoate | |
| CC\C═C/C1═CC2═C(C═CC═C2O)C(═O)O1 | Artemidinol | FooDB |
| [H][C@@]12[C@H](O)C(C)(C)CC[C@@]1(CC[C@]1(C)C2═CCC2[C@@]3 | 28- | FooDB |
| (C)CC[C@H](O[C@@H]4OC[C@H](O)[C@H](O)[C@H]4O)C(C)(C)C3CC | Glucosylsiaresinolate | |
| [C@@]12C)C(═O)O[C@@H]1O[C@H](CO)[C@@H](O)[C@H](O)[C@H]1O | 3-arabinoside | |
| CC(CCC(C)C(C)═C)C1CCC2(C)C3CCC4CC(═O)CCC44CC34CCC12C | 28- | FooDB |
| | Norcyclomusalenone | |
| CC1C2C(CC3C4CC═C5CC(OC6OC(CO)C(OC7OC(CO)C(O)C(OC8OC(C | Fistuloside C | FooDB |
| O)C(O)C(O)C8O)C7O)C(O)C6O)C(O)CC5(C)C4CCC23C)OC11CCC(C)C | | |
| O1 | | |
| CC(C(═O)C1═C(O)C(O)═C(O)C═C1)C1═CC═C(O)C═C1 | 3′-Hydroxy-O- | FooDB |
| | desmethylangolensin | |
| COC1═CC(C(═O)C(C)C2═CC═C(O)C═C2)═C(O)C═C1O | 5′-Methoxy-O- | FooDB |
| | desmethylangolensin | |
| CC(O)C#CC1═C(C)CC(CC1(C)C)OC1OC(CO)C(O)C(O)C1O | 5-Megastigmen-7- | FooDB |
| | yne-3,9-diol 3- | |
| | glucoside | |
| COC1═CC2═C(C═CC(═O)O2)C═C1C | 7-Methoxy-6- | FooDB |
| | methyl-2H-1- | |
| | benzopyran-2-one | |
| CC(C)═CCCC(C)(OC1OC(COC2OCC(O)C(O)C2O)C(O)C(O)C1O)C1CCC | Notoginsenoside | FooDB |
| 2(C)C1C(O)CC1C3(C)CCC(OC4OC(CO)C(O)C(O)C4OC4OC(CO)C(O)C | Fc | |
| (O)C4OC4OCC(O)C(O)C4O)C(C)(C)C3CCC21C | | |
| CC(C)C(C)CCC(C)C1CCC2(C)C3CCC4C(C)C(═O)CCC44CC34CCC12C | Cycloeucalanone | FooDB |
| CC(═O)OC1C(OC2OCC(O)C(O)C2O)C(OC2CCC3(C)C(CCC4(C)C3CC═C | Goyasaponin III | FooDB |
| 3C5CC(C)(C)CCC5(CCC43C)C(O)═O)C2(C)C)OC(C1OC1OC(CO)C(O)C | | |
| (O)C1O)C(O)═O | | |
| CC(C)═CC1OC2═C(C═CC(O)═C2)C2═C1C(═O)C1═C(O)C═C(O)C═C1O2 | Cyclocommunol | FooDB |
| CC(C)═CCCC(C)(OC1OC(COC2OC(CO)C(O)C(O)C2O)C(O)C(O)C1O)C1 | Notoginsenoside | FooDB |
| CCC2(C)C1C(O)CC1C3(C)CCC(OC4OC(CO)C(O)C(O)C4OC4OC(CO)C | Fa | |
| (O)C(O)C4OC4OCC(O)C(O)C4O)C(C)(C)C3CCC21C | | |
| OC(C1OC(═O)C(O)C1OC(═O)C1═CC(O)═C(O)C(O)═C1)C(O)═O | 3-O-Galloyl-1,4- | FooDB |
| | galactarolactone | |
| OC1COC2(OC3(O)CC45C2OC2(O)C4C4═C(OC32O)C(O)═C(O)C═C4C(═ | Putranjivain A | FooDB |
| O)OC2C(OC(═O)C3═CC(O)═C(O)C(O)═C3)OC3COC(═O)C4═CC(O)═C(O) | | |
| C(O)═C4C4═C(O)C(O)═C(O)C═C4C(═O)OC2C3OC5═O)C1O | | |
| [OH+]([Hg]C1═CC═CC═C1)[Hg]C1═CC═CC═C1 | m- | FooDB |
| | Hydroxydiphenyldimercury | |
| | (1+) | |
| OC1═CC2═C(C═C1)C1═C(C3═CC4═C(OCO4)C═C3O1)C(═O)O2 | Medicagol | FooDB |
| COC1═CC(═CC(O)═C1OC)C1CCC2═C(O1)C(O)═C(OC)C═C2C1═CC(OC)═ | 5′,5′″,8,8″- | FooDB |
| C(O)C2═C1CCC(O2)C1═CC(O)═C(OC)C(OC)═C1 | Tetrahydroxy- | |
| | 3′,3′″,4′,4′″,7′,7″- | |
| | hexamethoxy- | |
| | 5,5″-biflavan | |
| CC(C)═CC(═O)OC1C(OC2═C1C1═C(C═C2)C═CC(═O)O1)C(C)(C)OC(═O)\ | Edulisin I | FooDB |
| C═C\C1═CC═C(O)C═C1 | | |
| COC1═C([O−]) | 15-Epibetanidin 5- | FooDB |
| C═CC(\C═C\C(═O)OC2(CO)COC(OC3C(O)C(O)C(CO)OC3OC3═C(O)C═ | [E-feruloyl-(->3)- | |
| C4C(CC(C(O)═O)\[N+]4═C\C═C4/CC(NC(═C4)C(O)═O)C(O)═O)═C3)C2O)═ | apiosyl-(1->2)- | |
| C1 | glucoside] | |
| COC1═CC(OC)═C(C(═O)C2═CC═CC═C2)C(OC)═C1 | 2,4,6- | FooDB |
| | Trimethoxybenzophenone | |
| COC1═CC(CC═C)═CC(OC)═C1OC(C)C(OC(C)═O)C1═CC2═C(OCO2)C═C1 | 1-(2H-1,3- | FooDB |
| | Benzodioxol-5-yl)- | |
| | 2-[2,6-dimethoxy- | |
| | 4-(prop-2-en-1- | |
| | yl)phenoxy]propyl | |
| | acetate | |
| COC1═C2CC(COC2═CC2═C1CCC(C)(C)O2)C1═C(O)C(CC═C(C)C)═C(O) | Kanzonol H | FooDB |
| C═C1 | | |
| COC1═CC2═C(CC(CO2)C2═C3OC(C)(C)C═CC3═C(O)C═C2)C(OC)═C1C | Kanzonol I | FooDB |
| C═C(C)C | | |
| CN1CCCCC1CC(═O)C1═CC═C(O)C═C1 | Thalictroidine | FooDB |
| COC1═CC(═CC═C1O)C1OC(C(C)C1C)C1═CC(OC)═C(OC)C(OC)═C1 | Fragransin D1 | FooDB |
| COC1═C(O)C(O)═C2C(═O)C═C(OC2═C1)C1═CC(O)═C(O)C═C1 | 2-(3,4- | FooDB |
| | Dihydroxyphenyl)- | |
| | 5,6-dihydroxy-7- | |
| | methoxy-4H-1- | |
| | benzopyran-4-one | |
| COC(═O)CCC1(C)C2CC3═C(OC2(C)CC(O)C1C(C)(C)O)C(C)═C1COC(═O) | Austalide H | FooDB |
| C1═C3OC | | |
| CC(C)═CCC1═C(OC2═C(C(O)═C3CC(OC3═C2)C(C)(C)O)C1═O)C1═C(O) | Mulberranol | FooDB |
| C═C(O)C═C1 | | |
| OCC1OC(C(O)C(O)C1O)C1C2═C(C(O)═C(O)C═C2)C(═O)C2═C1C═C(CO) | 7-Hydroxyaloin B | FooDB |
| C═C2O | | |
| COC1═C(C═CC(O)═C1CC═C(C)C)C1COC2═C(C1)C═CC(O)═C2 | 4′,7-Dihydroxy-2′- | FooDB |
| | methoxy-3′- | |
| | prenylisoflavan | |
| CC12CC(O)CC3(OC1═O)C1CCC4(O)CC1(CC4═C)C(C23)C(O)═O | Gibberellin A81 | FooDB |
| CCCC1═CC(═O)OC2═C(C(═O)CC(C)C)C(O)═C3C4OOC(C)(C)C4OC3═C12 | 6-Hydroxy-9,9- | FooDB |
| | dimethyl-5-(3- | |
| | methyl-1- | |
| | oxobutyl)-1- | |
| | propyl-3H,9H- | |
| | [1,2]- | |
| | dioxolo[3′,4′:4,5]furo | |
| | [2,3- | |
| | f][1]benzopyran-3- | |
| | one | |
| C\C═C/SCC(N)C(O)═O | trans-S-(1- | FooDB |
| | Propenyl)-L- | |
| | cysteine | |
| CC(C)═CCC1═C2OC(C)(C)C═CC2═C(O)C2═C1OC1═C(C(OC3═C1C═C(O) | Cycloheterophyllin | FooDB |
| C(O)═C3)C═C(C)C)C2═O | | |
| CCC(CCC(C)C1CCC2C3═CCC4CC(O)CCC4(C)C3CCC12C)═C(C)C | Peposterol | FooDB |
| CC1═CC2═C(C(═O)C(C)(CO)C2)C(C)═C1CCOC1OC(CO)C(O)C(O)C1O | Pteroside A | FooDB |
| CC(C)═CCCC(COC1OCC(O)C(O)C1O)C1CCC2(C)C3═C(CCC12C)C1(C) | Tsugarioside B | FooDB |
| CCC(OC(C)═O)C(C)(C)C1CC3 | | |
| COC1═CC(═CC═C1O)C1═C(OC2OC(COC3OC(CO)C(O)C(O)C3O)C(O)C | Spinacetin 3- | FooDB |
| (O)C2O)C(═O)C2═C(O1)C═C(O)C(OC)═C2O | gentiobioside | |
| COC1═CC(CC(CO)C(CO)CC2═CC3═C(OCO3)C═C2)═CC(OC)═C1OC | Dihydroclusin | FooDB |
| COC1C═C2C(CCC(OC3OCC(O)C(O)C3O)C2(C)C)C2(C)CCC3(C)C(C(C)C | Hebevinoside XI | FooDB |
| CC═C(C)C)C(CC3(C)C12)OC1OC(COC(C)═O)C(OC(C)═O)C(O)C1O | | |
| COC1═C(C(O)═C(C(═O)C2═CC═CC═C2)C(O)═C1)C1═CC(O)═C2OC3═C | Garciduol A | FooDB |
| (C═CC═C3O)C(═O)C2═C1O | | |
| OCC1═CNC2═NC(═O)NC(═O)C2═N1 | 6- | FooDB |
| | (Hydroxymethyl)- | |
| | 2,4(1H,3H)- | |
| | pteridinedione | |
| COC1═C(O)C═CC(CCC(O)CC(═O)CCC2═CC═CC═C2)═C1 | 5-Hydroxy-7-(4- | FooDB |
| | hydroxy-3- | |
| | methoxyphenyl)-1- | |
| | phenyl-3- | |
| | heptanone | |
| OCC1OC(OC2═CC(O)═CC3═[O+]C(═C(OC4OC(COC(═O)\C═C\C5═CC(O)═ | YGM 1A | FooDB |
| C(O)C═C5)C(O)C(O)C4OC4OC(COC(O)═C5C═CC(═O)C═C5)C(O)C(O)C | | |
| 4O)C═C23)C2═CC(O)═C(O)C═C2)C(O)C(O)C1O | | |
| OCC1OC2C(OC(═O)N2C2═CC═CC═C2O)C(O)C1O | Hexahydro-6,7- | FooDB |
| | dihydroxy-5- | |
| | (hydroxymethyl)- | |
| | 3-(2- | |
| | hydroxyphenyl)- | |
| | 2H-pyrano[2,3- | |
| | d]oxazol-2-one | |
| C\C═C(/C)C(═O)OC1C(O)C(C)(C)CC2C3═CCC4C5(C)CCC(O)C(C)(C═O) | (3b,16a,21b,22a)- | FooDB |
| C5CCC4(C)C3(C)CC(O)C12CO | 12-Oleanene- | |
| | 3,16,21,23,28- | |
| | pentol-22- | |
| | angeloyloxy-23-al | |
| COC1═C(OC)C═C(C═C1)C1═C(O)C(═O)C2═C(O)C═C(O)C═C2O1 | 3′,4′-Di-O- | FooDB |
| | methylquercetin | |
| CC1═CC(═O)C2C(C)(C)C(O)CCC2(C)C1COC1═CC2═C(C═CC(═O)O2)C═ | (3′x,5′a,9′x,10′b)- | FooDB |
| C1 | O-(3-Hydroxy-6- | |
| | oxo-7-drimen-11- | |
| | yl)umbelliferone | |
| CC12CCCC(C)(C1C(O)C(O)C13CC(O)(COC4OC(CO)C(O)C(O)C4O)C(O) | Canavalioside | FooDB |
| (C1)CCC23)C(O)═O | | |
| COC1═C(O)C═CC(═C1)C1OC2═C(C═C(C═C2O)C2OC3═CC(O)═CC(O)═C | Silicristin | FooDB |
| 3C(═O)C2O)C1CO | | |
| COC1═C(O)C2═C(C(CC═C(C)C)═C1)C(═O)C1═C(O2)C(CC═C(C)C)═C(O | Dulxanthone C | FooDB |
| C)C═C1O | | |
| CCCCCCCCCCCCC(O)C1CCC(O1)C(O)CCCC(═O)CCCCCCC(O)CC1═C | Reticulacinone | FooDB |
| C(C)OC1═O | | |
| COC1═C(O)C═CC(\C═C\C(═O)OC(C(O)═O)C(O)(CC(O)═O)C(O)═O)═C1 | 2-O- | FooDB |
| | Feruloylhydroxycitric | |
| | acid | |
| OCCCCCCCC\C═C\C(O)C#CC#C | 9-Pentadecene- | FooDB |
| | 12,14-diyne-1,11- | |
| | diol | |
| OCC1OC(OC2C(O)C(O)C(CO)OC2C2═C(O)C3═C(OC(═CC3═O)C3═CC═ | Meloside L | FooDB |
| C(O)C(O)═C3)C═C2O)C(O)C(O)C1O | | |
| COC1═C(O)C═C(O)C2═C1OC(C1═CC═C(O)C═C1)═C(OC1OC(CO)C(O)C | Sexangularetin 3- | FooDB |
| (O)C1OC1OC(CO)C(O)C(O)C1O)C2═O | sophoroside | |
| CC1OC(OC2C(OC3CCC4(C)C(CCC5(C)C4CCC4C6C7(CC54CO7)OC(CC | Hoduloside V | FooDB |
| 6(C)O)C═C(C)C)C3(C)C)OC(CO)C(O)C2OC2OC(CO)C(O)C(O)C2O)C(O) | | |
| C(O)C1O | | |
| COC1═C(O)C═CC(\C═C\C(═O)OCCCCCCCCCCCCCCCCCCCCCCCCCC | 1,26- | FooDB |
| OC(═O)\C═C\C2═CC(OC)═C(O)C═C2)═C1 | Hexacosanediol | |
| | diferulate | |
| CCCCCCCCCCCC(O)CCCCCCCCCCCCCCCCCO | (+)-1,18- | FooDB |
| | Nonacosanediol | |
| CC(OC1OC(CO)C(O)C(O)C1O)\C═C\C1C(C)CC(═O)CC1(C)C | 9-Hydroxy-7- | FooDB |
| | megastigmen-3- | |
| | one glucoside | |
| CCCCCCCCCCCCCCCC(═O)NCC1═CC═CC═C1 | Macamide B | FooDB |
| NC(═O)CC1═CC═CC2═C1C═CC═C2 | 2-(1- | FooDB |
| | Naphthyl)acetamide | |
| COC1═C(O)C═C2C3OC(O)C4═C(C═CC5═C4OCO5)C3N(C)CCC2═C1 | N-Methyl-14-O- | FooDB |
| | demethylepiporphyroxine | |
| CC1C2C3CCC4C5(C)CCC(O)C(C)(C)C5CCC4(C)C3(C)CC(O)C2(C)C(O)C═ | Heliantriol C | FooDB |
| C1C | | |
| CC(═C)C1CCC(C)(O)C2CCC(C)(O)C2C1 | Guaidiol | FooDB |
| OC(═O)COC1═CC2═CC═CC═C2C═C1 | (2- | FooDB |
| | Naphthalenyloxy)acetic | |
| | acid | |
| CCC(SSCC═C)S(═O)\C═C\C | 2-Propenyl 1-(1- | FooDB |
| | propenylsulfinyl)propyl | |
| | disulfide | |
| CC(C)═CCC1═C2CCC3═CC(O)═C(O)C═C3C2═C(O)C═C1O | Gancaonin V | FooDB |
| OC1OC(COC2OC(COC3OCC(O)C(O)C3O)C(O)C2O)C(O)C1O | beta-D- | FooDB |
| | Xylopyranosyl-(1-> | |
| | 5)-alpha-L- | |
| | arabinofuranosyl- | |
| | (1->5)-L-arabinose | |
| CC1OC(OCC2OC(OC3═C(OC4═CC(O)═CC(O)═C4C3═O)C3═CC═C(O)C═ | Biorobin | FooDB |
| C3)C(O)C(O)C2O)C(O)C(O)C1O | | |
| CC1(C)C(CCC2(C)C1CCC1(C)C2C(═O)C═C2C3CC(C)(CCC3(C)CCC12C) | Licoricesaponin | FooDB |
| C(═O)OC1OC(CO)C(O)C(O)C1O)OC1OC(C(O)C(O)C1OC1OC(C(O)C(O) | A3 | |
| C1O)C(O)═O)C(O)═O | | |
| CC(C)═CCC(O)C1═CC(═O)C2═C(O)C═CC(O)═C2C1═O | C.I. Natural Red | FooDB |
| | 20 | |
| COC1═C(O)C═CC(═C1)C1═[O+]C2═C(C═C1O[C@@H]1O[C@H](COC(C)═O) | Peonidin 3-O- | FooDB |
| [C@@H](O)[C@H](O)[C@H]1O)C(O)═CC(O)═C2 | (acetylglucoside) | |
| CC1OC(OC2═C(OC3═C(C(O)═CC(OC4OC(CO)C(O)C(O)C4O)═C3)C2═O) | quercetin 3-O- | FooDB |
| C2═CC═C(O)C(O)═C2)C(O)C(O)C1O | rhamnoside-7-O- | |
| | glucoside | |
| OCC1OC(C(O)C(O)C1O)C1═C(O)C═C2OC(═CC(═O)C2═C1O)C1═CC═C | isovitexin | FooDB |
| (O)C═C1 | | |
| CCCCCCCCCC([O−])═O | decanoate | FooDB |
| CCCCCCCCCCCCCCCCCC(O)CCCCCCCCCCC | Nonacosan-13-ol | FooDB |
| OCC(O)C1OC2C(OC(═O)C3═C2C(O)═C(O)C(O)═C3C2═C(O)C(O)═C3OC | Castacrenin A | FooDB |
| (═O)C4═CC(O)═C(O)C5═C4C3═C2C(═O)O5)C1O | | |
| COC1═CC(CC\C═C\C(═O)CCC2═CC(OC)═C(O)C(OC)═C2)═CC═C1O | Isogingerenone B | FooDB |
| OCC1OC(OCC2OC(OC3═C(OC4═CC(O)═CC(O)═C4C3═O)C3═CC═C(O)C═C3) | Kaempferol 3-(2G- | FooDB |
| C(OC3OC(CO)C(O)C(O)C3O)C(O)C2O)C(O)C(O)C1O | glucosylgentiobioside) | |
| CC\C═C/C1═CC2═C(C═CC═C2)C(═O)O1 | (Z)-3-(1-Butenyl)- | FooDB |
| | 1H-2-benzopyran- | |
| | 1-one | |
| CC\C═C/C[C@@H]1[C@H](CCCCCC(O)═O)CCC1═O | OPC-6:1O | FooDB |
| [H][C@]12CCC(═C)[C@@]1([H])[C@@]13OC(═O)C(CCO[C@@H]4O[C@ | Cichorioside J | FooDB |
| H](CO)[C@@H](O)[C@H](O)[C@H]4O)═C1C═C[C@]2(O)CO3 | | |
| CC(═O)O[C@H]1[C@]2(C)CC[C@@H](C2)C1(C)C | (+)-beta-Fenchyl | FooDB |
| | acetate | |
| CC(C)═CCC1═C(O)C═C2OC3═C(C(═O)C2═C1O)C(CCC(C)(C)O)═C(O)C | Garcinone C | FooDB |
| (O)═C3 | | |
| COC1═C(O)C═CC(═C1)C1═[O+]C2═C(C═C1O[C@@H]1O[C@H](CO[C@ | Peonidin 3- | FooDB |
| @H]3OC(C)[C@H](OC(═O)\C═C\C4═CC(O)═C(O)C═C4)[C@@H](O)[C@H] | caffeoyl-rutinoside | |
| 3O)[C@@H](O)[C@H](O)[C@H]1O)C(O[C@@H]1O[C@H](CO)[C@@H] | 5-glucoside | |
| (O)[C@H](O)[C@H]1O)═CC(O)═C2 | | |
| [H]OC([H])([H])C([H])(OC([H])([H])C([H])([H])C([H])([H])C([H])([H])C(\[H])═C | 2-Arachidonyl | FooDB |
| (\[H])C([H])([H])C(\[H])═C(\[H])C([H])([H])C(\[H])═C(\[H])C([H])([H])C(\[H])═C(\ | glycerol ether | |
| [H])C([H])([H])C([H])([H])C([H])([H])C([H])([H])C([H])([H])[H])C([H])([H])O[H] | | |
| CC\C═C/C1═CC2═C(C═CC═C2)C(═O)O1 | (Z)-3-(1-Butenyl)- | FooDB |
| | 1H-2-benzopyran- | |
| | 1-one | |
| OCC1OC(OC2═C(OC3═C(C(O)═CC(O)═C3)C2═O)C2═CC(O)═C(O)C(O)═ | Theaflavonin | FooDB |
| C2C2═C(C═C(O)C(O)═C2O)C2OC3═CC(O)═CC(O)═C3CC2OC(═O)C2═C | | |
| C(O)═C(O)C(O)═C2)C(O)C(O)C1O | | |
| COC1═CC2═C(C(═O)C═C(O2)C2═CC═C(O)C═C2)C(O)═C1C | \4′,5-Dihydroxy-7- | FooDB |
| | methoxy-6- | |
| | methylflavone\ | |
| COC1═CC(═CC═C1O)C1═C(OC2OC(COC3OC(C)C(O)C(O)C3O)C(O)C(O) | Isorhamnetin 3- | FooDB |
| C2OC2OCC(O)(CO)C2O)C(═O)C2═C(O1)C═C(O)C═C2O | (2G- | |
| | apiosylrutinoside) | |
| COC1═C(C)C(O)═C2C(═O)C═C(C)OC2═C1 | Eugenitin | FooDB |
| CCCCCCCCCCCCCCCCCCC(O)CCCCCCCCCCCCC | Dotriacontan-14-ol | FooDB |
| CC(C)═CCC\C(C)═C\C═C\C(\C)═C\C═C\C(\C)═C\C═C/C═C(\C)/C═C/C═C(\ | (15Z)-Lycopene | FooDB |
| C)/C═C/C═C(\C)CCC═C(C)C | | |
| OC(═O)C1CSC(═S)C1 | 2- | FooDB |
| | Thioxothiazolidine- | |
| | 4-carboxylic acid | |
| COC(═O)NC1═NC2═C(N1)C═CC═C2 | Carbendazim | FooDB |
| COC1═C(O)C═CC(\C═C\C(═O)NCCC2═CNC3═C2C(═C(O)C═C3)C2═C3C | \4,4″-bis(N- | FooDB |
| (CCNC(═O)\C═C\C4═CC(OC)═C(O)C═C4)═CNC3═CC═C2O)═C1 | feruloyl)serotonin\ | |
| CC(═O)C1═C([O−])OC(C)═CC1═O | Sodium | FooDB |
| | dehydroacetate | |
| CCC1═C(C)C═C(C)C═C1 | \1,3-Dimethyl-4- | FooDB |
| | ethylbenzene\ | |
| OC1CC(O)(CC(C1O)C(═O)OC1═C(C(O)═C(O)C(O)═C1)C1═C(C═C(O)C(O)═ | Theogallinin | FooDB |
| C1O)C1OC2═CC(O)═CC(O)═C2CC1OC(═O)C1═CC(O)═C(O)C(O)═C1)C | | |
| (O)═O | | |
| OC(═O)C1═CC(O)═C(O)C(OC2═C3C(═O)OC4═C5C(═CC(O)═C4O)C(═O) | Sanguisobic acid | FooDB |
| OC(C(O)═C2O)═C35)═C1 | dilactone | |
| CCCCCCCCCCCCGCCCCCCC(═O)CCCCCCCCCCCCC | Tritriacontan-14- | FooDB |
| | one | |
| COC1═C(O)C═C(\C═C\C(═O)OC[C@H]2O[C@@H](OC3═CC4═C(C═C(O) | Peonidin 3- | FooDB |
| C═C4O[C@@H]4O[C@H](CO)[C@@H](O)[C@H](O)[C@H]4O)[O+]═C3C3═ | feruloyl- | |
| CC(OC)═C(O)C═C3)[C@H](O[C@@H]3O[C@H](CO)[C@@H](O)[C@H] | digiucoside 5- | |
| (O)[C@H]3O)[C@@H](O)[C@@H]2O)C═C1 | glucoside | |
| CC(C)(O)C(O)CC(═O)C(C)(O)C1C(O)CC2(C)C3CC═C4C(CC(═O)C(═O)C4 | Cucurbitacin K | FooDB |
| (C)C)C3(C)C(═O)CC12C | | |
| CC(C)═CCC1═CC(═O)C(C)═C(C)C1═O | Plastoquinone | FooDB |
| OC1═CC(═CC(O)═C1O)C(═O)OC1OC2COC(═O)C3═C(C(O)═C(O)C(OC4═ | Rugosin D | FooDB |
| C(C═C(O)C(O)═C4O)C(═O)OC4OC5COC(═O)C6═CC(O)═C(O)C(O)═C6C6═ | | |
| C(C═C(O)C(O)═C6O)C(═O)OC5C(OC(═O)C5═CC(O)═C(O)C(O)═C5)C4O | | |
| C(═O)C4═CC(O)═C(O)C(O)═C4)═C3)C3═C(C═C(O)C(O)═C3O)C(═O)OC2 | | |
| C(OC(═O)C2═CC(O)═C(O)C(O)═C2)C1OC(═O)C1═CC(O)═C(O)C(O)═C1 | | |
| CC1(C)CCC2(C)C(O)CC3(C)C(CCC4C5(C)CCC(O)C(C)(C)C5CCC34C)═C | Coflodiol | FooDB |
| 2C1 | | |
| OC[C@H]1O[C@@H](OC2═CC3═C(C([C@H](O3)C3═CC═C(O)C═C3)C3═ | Foeniculoside XI | FooDB |
| CC(O)═CC4═C3[C@H]([C@@H](O4)C3═CC═C(O)C═C3)C3═CC(O)═CC(O | | |
| [C@@H]4O[C@H](CO)[C@@H](O)[C@H](O)[C@H]4O)═C3)C(\C═C/C3═C | | |
| C═C(O)C═C3)═C2)[C@H](O)[C@@H](O)[C@@H]1O | | |
| CCC(C)NC(═O)\C═C\CCCCCC\C═C\C1═CC2═C(OCO2)C═C1 | Piperchabamide D | FooDB |
| COC1═CC═C(\C═C/C)C═C1O | Isochavibetol | FooDB |
| COC1═CC═C(C═C1)C1═COC2═C(C═CC(OC3OC(COC(═O)CC(O)═O)C(O) | Formononetin 7- | FooDB |
| C(O)C3O)═C2)C1═O | (6″- | |
| | malonylglucoside) | |
| COC1═CC(\C═C\C(═O)OC2C(CO)OC(OCC3OC(OC4═CC5═C(C═C(O)C═ | Alatanin 1 | FooDB |
| C5O)[O+]═C4C4═CC(O)═C(O)C═C4)C(O)C(O)C3O)C(O)C2O)═CC(OC)═C | | |
| 1O | | |
| COC1═C(O)C═CC(═C1)C1═[O+]C2═C(C═C1O[C@@H]1O[C@H](COC(═O) | Peonidin 3-(6″- | FooDB |
| CC(O)═O)[C@@H](O)[C@H](O)[C@H]1O)C(O[C@@H]1O[C@H](CO)[C | malonyl- | |
| @@H](O)[C@H](O)[C@H]1O)═CC(O)═C2 | glucoside) 5- | |
| | glucoside | |
| [H]OC([H])([H])C([H])(N([H])C(═O)C([H])([H])C([H])([H])C([H])([H])C([H])([H]) | Palmitoyl serinol | FooDB |
| C([H])([H])C([H])([H])C([H])([H])C([H])([H])C([H])([H])C([H])([H])C([H])([H])C | | |
| ([H])([H])C([H])([H])C([H])([H])C([H])([H])[H])C([H])([H])O[H] | | |
| C[C@@H]1O[C@@H](O[C@@H]2[C@H](O)[C@H](O)[C@H](C)OC2C2═ | 2″-O-alpha-L- | FooDB |
| C(O)C3═C(OC(═CC3═O)C3═CC═C(O)C(O)═C3)C═C2O)[C@H](O)[C@H] | Rhamnosyl-6-C- | |
| (O)[C@H]1O | fucosyl-luteolin | |
| COC1═CC(═CC═C1O)C1CC(═O)C2═C(O)C═C(O)C═C2O1 | Homoeriodictyol | FooDB |
| CC\C═C/C[C@@H]1[C@H](CCCC(O)═O)CCC1═O | OPC-4:0 | FooDB |
| CC1OC(OC2═CC3═C(C(O)═C2)C(═O)C(OC2OC(C)C(O)C(O)C2O)═C(O3) | \Quercetin 3,7- | FooDB |
| C2═CC═C(O)C(O)═C2)C(O)C(O)C1O | dirhamnoside\ | |
| COC1═CC═C(C═C1OC)C1═C(OC)C(═O)C2═C(O1)C═C(O)C(OC)═C2OC | \7-Hydroxy- | FooDB |
| | 3,3′,4′,5,6- | |
| | pentamethoxyflavone\ | |
| COC(═O)C1═C(CC(C)C)C(C2═NCCS2)═C(N═C1C(F)F)C(F)(F)F | Thiazopyr | FooDB |
| [H]OC([H])([H])C([H])([H])N([H])C(═O)C([H])([H])C([H])([H])C(\[H])═C(\[H])C | Docosahexaenoyl | FooDB |
| ([H])([H])C(\[H])═C(\[H])C([H])([H])C(\[H])═C(\[H])C([H])([H])C(\[H])═C(\[H])C | Ethanolamide | |
| ([H])([H])C(\[H])═C(\[H])C([H])([H])C(\[H])═C(\[H])C([H])([H])C([H])([H])[H] | | |
| OC[C@H]1O[C@@H](OC2═CC(O)═C3C(═O)C(O)═C(OC3═C2)C2═CC═C | Quercetin 7- | FooDB |
| (O)C(O)═C2)[C@H](O)[C@@H](O)[C@@H]1O | glucoside | |
| CC(C)C(═C)CC[C@@H](C)[C@H]1CC[C@@]2(C)C3CC[C@H]4C(C)[C@ | 24-Methylene-31- | FooDB |
| @H](O)CC[C@]4(C)C3═CC[C@]12C | nor-5alpha-lanost- | |
| | 9(11)-3beta-ol | |
| OC1C(COC(═O)\C═C\C2═CC═C(O)C═C2)OC(OC2═CC3═C(C═C(O)C═C3 | Delphinidin 3-(6-p- | FooDB |
| O)[O+]═C2C2═CC(O)═C(O)C(O)═C2)C(O)C1O | coumaroylglucoside) | |
| [H]OC([H])(C(═O)C([H])([H])[H])C1([H])[N([H])C2═C(N═C(N([H])[H])N([H])C2═O) | 1-Hydroxy-2- | FooDB |
| N([H])C1([H])[H] | oxopropyl | |
| | tetrahydropterin | |
| [H][C@]1(NC(═O)[C@@H]2CCCN2C(═O)[C@@H](CC(C)(C)O)OC(═O)CC | Hydroxyhomodestruxin B | FooDB |
| NC(═O)[C@H](C)N(C)C(═O)[C@H](C(C)CC)N(C)C1═O)[C@@H](C)CC | | |
| CC1(C)CC2CC1CC2═C | beta-Fenchene | FooDB |
| CC1═CCC(CC1)C(C)(O)CO | \1-p-Menthene- | FooDB |
| | 8,9-diol\ | |
| OCC1OC(OC2═CC(O)═CC3═C2C═C(OC2OC(CO)C(O)C(O)C2OC2OCC | Delphinidin 3- | FooDB |
| (O)C(O)C2O)C(═[O+]3)C2═CC(O)═C(O)C(O)═C2)C(O)C(O)C1O | sambubioside 5- | |
| | glucoside | |
| COC1═C(O)C═CC(═C1)C1═[O+]C2═C(C═C1O[C@@H]1O[C@H](COC(═O) | Peonidin 3-(6″- | FooDB |
| \C═C\C3═CC(O)═C(O)C═C3)[C@@H](O)[C@H](O)[C@H]1O)C(O)═CC(O)═C2 | caffeoyl- | |
| | glucoside) | |
| CCCCNC(═O)N1C(NC(═O)OC)═NC2═C1C═CC═C2 | Benomyl | FooDB |
| COC1═CC(═CC(OC)═C1O)C1═[O+]C2═C(C═C1OC1O[C@H](COC(C)═O) | Malvidin 3-(6″- | FooDB |
| [C@H](O)[C@H](O)[C@H]1O)C(O)═CC(O)═C2 | acetyl- | |
| | galactoside) | |
| CC(C)[C@@H]1C[C@H](O)[C@@H](C)C═C1 | (−)-trans- | FooDB |
| | Isomenth-5-en-2- | |
| | ol | |
| CC1═C(O)C(O)═CC(═C1)[C@@]12OC3═CC(O)═CC(O)═C3[C@@H]([C@ | Proanthocyanidin | FooDB |
| H]1O)C1═C3O[C@@H]([C@H](O)CC3═C(O)C═C1O2)C1═CC═C(O)C(O)═ | A2 | |
| C1 | | |
| COC1═C(O)C═CC(\C═C\C(═O)NCCC2═CNC3═C2C(═C(O)C═C3)C2═C3C | 4-[N-(p- | FooDB |
| (CCNC(═O)\C═C\C4═CC═C(O)C═C4)═CNC3═CC═C2O)═C1 | Coumaroyl)serotonin- | |
| | 4″-yl]-N- | |
| | feruloylserotonin | |
| COC1═C2OC(C(CO)C2═CC(CC2COC(═O)C2CC2═CC(OC)═C(O)C(═C2)C | Arctignan E | FooDB |
| (CO)C(O)C2═CC═C(O)C(OC)═C2)═C1)C1═CC═C(O)C(OC)═C1 | | |
| CCCCCCCCCCCCCCCCC(O)CCCCCCCCCCCCCCC | Dotriacontan-16-ol | FooDB |
| [H]OC(═O)C([H])(N([H])[H])C([H])([H])C(═O)N([H])C([H])(C(═O)O[H])C([H]) | L-beta-Aspartyl-L- | FooDB |
| ([H])[H] | alanine | |
| OC[C@H]1O[C@@H](OC2═CC3═C(C(O)═C2)C(═O)C(O[C@@H]2O[C@H] | \Quercetin 3,7- | FooDB |
| (CO)[C@@H](O)[C@H](O)[C@H]2O)═C(O3)C2═CC(O)═C(O)C═C2)[C@H] | diglucoside\ | |
| (O)[C@@H](O)[C@@H]1O | | |
| CCCCCCCCC[C@@H](CC(═O)CCC1═CC═C(C)C(OC)═C1)OC(C)═O | Acetoxy-[10]- | FooDB |
| | gingerol | |
| COC1═C(OC)C2═CC(CCCCC(O)CCC3═CC═C(O)C2═C3)═C1OC1OC(CO) | Myricanol 5- | FooDB |
| C(O)C(O)C1O | glucoside | |
| CC1═CC[C@@H](O)[C@]2(C)CC[C@@H]3[C@H](OC(═O)C3═C)[C@@H] | Santamarin | FooDB |
| 12 | | |
| [H][C@@]12C[C@H](O)[C@]3(O)C[C@]1(CC3═C)[C@@H](C(O)═O)[C@] | Gibberellin A87 | FooDB |
| 1([H])[C@]3(C)[C@@H](O)C═C[C@@]21OC3═O | | |
| OC1C(O)C(COC(═O)\C═C\C2═CC═C([O−]) | Isogomphrenin II | FooDB |
| C═C2)OC(OC2═C(O)C═C3CC(C(O)═O)\[N+](═C\C═C4/CC(NC(═C4)C(O)═O) | | |
| C(O)═O)C3═C2)C1O | | |
| COC1═CC(O)═C2C(═O)C(O)═C(OC2═C1)C1═CC═C(O)C═C1 | Rhamnocitrin | FooDB |
| CCCCCCCCCCCCCC(O)CC(═O)CCC1═CC═C(O)C(OC)═C1 | [14]-Gingerol | FooDB |
| CC1CC2═CC(C)═C(CCO)C(C)═C2C1═O | (R)-Pterosin B | FooDB |
| OC1OC2COC(═O)C3═CC(O)═C(O)C(O)═C3C3═C(O)C(O)═C(O)C═C3C(═ | Tellimagrandin I | FooDB |
| O)OC2C(OC(═O)C2═CC(O)═C(O)C(O)═C2)C1OC(═O)C1═CC(O)═C(O)C | | |
| (O)═C1 | | |
| COC1═CC═C(C═C1)C1═COC2═C(C═CC(OC3OC(COC(═O)CC(O)═O)C(O) | Formononetin 7- | FooDB |
| C(O)C3O)═C2)C1═O | (6″- | |
| | malonylglucoside) | |
| COC1═C(OC2═C(C(O)═CC(O)═C2)C1═O)C1═CC═CC═C1 | 3-Methylgalangin | FooDB |
| OCC(OC1OCC(O)C(O)C1O)C(O)C(O)C═O | 4-O-alpha-D- | FooDB |
| | Xylopyranosyl-D- | |
| | xylose | |
| CCCCCCCCCCCCCCCCCCCCCCCC(═O)CCCCCCCCC | Tritriacontan-10- | FooDB |
| | one | |
| CC12CC(C3═C(C4═C(O3)C═C(C═C4OC(═O)C3═CC═C(O)C═C3O)C3═CC | Mulberrofuran M | FooDB |
| 4═C(O3)C═C(O)C═C4)C1═O)C1═CC═C(O)C═C1O2 | | |
| COC1═CC2═C(C═C1OC)C1═C3C(CCN(C)C3═C2C═O)═C(OC)C(OC)═C1OC | 7- | FooDB |
| | Formyldehydrothalicsimidine | |
| NC(═N)NCCCCNC(═O)\C═C/C1═CC2═C(OC(C2C(═O)NCCCCNC(N)═N)C | Hordatine A | FooDB |
| 2═CC═C(OC3OC(CO)C(O)C(O)C3O)C═C2)C(O)═C1 | glucoside | |
| COC1═CC(═CC═C1O)C(O)C(CO)OC1OC(CO)C(O)C(O)C1O | \(1x,2x)- | FooDB |
| | Guaiacylglycerol | |
| | 2-glucoside\ | |
| COC1═CC(═CC═C1O)[C@H]1OC[C@H]2[C@@H]1CO[C@@H]2C1═CC | Medioresinol | FooDB |
| (OC)═C(O)C(OC)═C1 | | |
| COC1═C(O)C═CC(═C1)C1═[O+]C2═C(C═C1O[C@@H]1O[C@H](COC(C)═O) | \Peonidin acetyl | FooDB |
| [C@@H](O)[C@H](O)[C@H]1O)C(O[C@@H]1O[C@H](CO)[C@@H] | 3,5-diglucoside\ | |
| (O)[C@H](O)[C@H]1O)═CC(O)═C2 | | |
| CC(C)CNC(═O)\C═C\CCC1═CC2═C(OCO2)C═C1 | \4,5- | FooDB |
| | Dihydropiperlonguminine\ | |
| [H]O[C@]([H])(C([H])([H])C([H])([H])[C@@]([H])(C([H])([H])[H])C1([H])C([H]) | \(24R)-Cholest-5- | FooDB |
| ([H])C([H])([H])[C@@]2([H])[C@]3([H])C([H])([H])C([H])═C4C([H])([H])[C@ | ene-3-beta,24- | |
| @]([H])(O[H])C([H])([H])C([H])([H])[C@]4(C([H])([H])[H])[C@@]3([H])C([H]) | diol\ | |
| ([H])C([H])([H])[C@]12C([H])([H])[H])C([H])(C([H])([H])[H])C([H])([H])[H] | | |
| [H]C(O)(CO)COC(═O)\C═C\C1═CC═C(O)C═C1 | (S)-(+)-1-(p- | FooDB |
| | Hydroxy-trans- | |
| | cinnamoyl)- | |
| | glycerol | |
| [H]OC([H])(C(═O)N([H])C([H])([H])C([H])([H])C(═O)N([H])C([H])([H])C([H]) | (S)- | FooDB |
| ([H])SC(═O)C([H])([H])C([H])(O[H])C([H])([H])C([H])([H])C([H])([H])C([H])([H]) | Hydroxyoctadecanoyl- | |
| C([H])([H])C([H])([H])C([H])([H])C([H])([H])C([H])([H])C([H])([H])C([H])([H])C | CoA | |
| ([H])([H])C([H])([H])C([H])([H])C([H])([H])[H])C(C([H])([H])[H])(C([H])([H])[H])C | | |
| ([H])([H])OP(═O)(O[H])OP(═O)(O[H])OC([H])([H])C1([H])OC([H])(N2C([H])═ | | |
| NC3═C2C([H])═C([H])N═C3N([H])[H])C([H])(O[H])C1([H])OP(═O)(O[H])O[H] | | |
| [H][C@]1(CC(═O)OCCC2═CC(O)═C(O)C═C2)\C(═C/C)[C@H](O[C@@H]2 | Demethyloleuropein | FooDB |
| O[C@H](CO)[C@@H](O)[C@H](O)[C@H]2O)OC═C1C(O)═O | | |
| OC1OC2COC(═O)C3═C(C(O)═C(O)C(OC4═C(C═C(O)C(O)═C4O)C(═O)O | Rugosin E | FooDB |
| C4OC5COC(═O)C6═CC(O)═C(O)C(O)═C6C6═C(C═C(O)C(O)═C6O)C(═O) | | |
| OC5C(OC(═O)C5═CC(O)═C(O)C(O)═C5)C4OC(═O)C4═CC(O)═C(O)C(O)═ | | |
| C4)═C3)C3═C(C═C(O)C(O)═C3O)C(═O)OC2C(OC(═O)C2═CC(O)═C(O)C | | |
| (O)═C2)C1OC(═O)C1═CC(O)═C(O)C(O)═C1 | | |
| OCC1OC(C(O)C(O)C1O)C1═C2OC(═CC(═O)C2═C(O)C═C1O)C1═CC═C | Vitexin | FooDB |
| (O)C═C1 | | |
| COC1═C(O)C2═C(C═C1)C1OC3═CC(O)═CC═C3C1CO2 | Melilotocarpan B | FooDB |
| OC1C(OC2═C(C1C1═C(O)C═C(O)C3═C1OC(C(OC(═O)C1═CC(O)═C(O)C | \Epicatechin- | FooDB |
| (O)═C1)C3C1═C(O)C═C(O)C3═C1OC(C(C3)OC(═O)C1═CC(O)═C(O)C(O)═ | (4beta->8)-ent- | |
| C1)C1═CC═C(O)C(O)═C1)C1═CC═C(O)C(O)═C1)C(O)═CC(O)═C2)C1═C | epicatechin- | |
| C═C(O)C(O)═C1 | (4alpha->8)-ent- | |
| | epicatechin 3′,3″- | |
| | digallate\ | |
| COC1═CC(Cl)═C2NC(═O)C(O[C@@H]3O[C@H](CO)[C@@H](O)[C@H] | \5-Chloro-2- | FooDB |
| (O)[C@H]3O)OC2═C1 | hydroxy-7- | |
| | methoxy-2H-1,4- | |
| | benzoxazin-3(4H)- | |
| | one beta-D- | |
| | glucopyranoside\ | |
| COC1═CC(OC)═CC(O)═C1 | 3,5- | HMDB |
| | Dimethoxyphenol | |
| CC1═C(Cl)C(O)═CC═C1NC1═C(C═CC═C1)C(O)═O | N-(2-methyl-3- | HMDB |
| | chloro-4- | |
| | hydroxyphenyl)anthranilic | |
| | acid | |
| O(═O)(═O)OC(═O)C1═CNC2═C1C═CC═C2 | Indole-3-carboxilic | HMDB |
| | acid-O-sulphate | |
| OC[C@H]1O[C@H](O[C@H]2[C@H](O)[C@@H](O)[C@@H](O[C@H]3[C | Maltotridecaose | HMDB |
| @H](O)[C@@H](O)[C@@H](O[C@H]4[C@H](O)[C@@H](O)[C@@H](O | | |
| [C@H]5[C@H](O)[C@@H](O)[C@@H](O[C@H]6[C@H](O)[C@@H](O)[C | | |
| @@H](O[C@H]7[C@H](O)[C@@H](O)[C@@H](O[C@H]8[C@H](O)[C@ | | |
| @H](O)[C@@H](O[C@H]9[C@H](O)[C@@H](O)[C@@H](O[C@H]%10[C | | |
| @H](O)[C@@H](O)[C@@H](O[C@H]%11[C@H](O)[C@@H](O)[C@@H] | | |
| (O[C@H]%12[C@H](O)[C@@H](O)[C@@H](O[C@H]%13[C@H](O)[C@@H] | | |
| (O)[C@@H](O)O[C@@H]%13CO)O[C@@H]%12CO)O[C@@H]%11CO) | | |
| O[C@@H]%10CO)O[C@@H]9CO)O[C@@H]8CO)O[C@@H]7CO)O[C@ | | |
| @H]6CO)O[C@@H]5CO)O[C@@H]4CO)O[C@@H]3CO)O[C@@H]2CO) | | |
| [C@H](O)[C@@H](O)[C@@H]1O | | |
| C[C@@H](O)[C@H](N)C(═O)NC(CCCCN)C(═O)N1CCC[C@H]1C(═O)N[C | Tuftsin | HMDB |
| @@H](CCCNC(N)═N)C(O)═O | | |
| NS(═O)(═O)C1═C(Cl)C═C(NCC2═CC═CS2)C(═C1)C1═NNN═N1 | Azosemide | HMDB |
| COC(═O)\C═C\C(\C)═C\C═C\C(\C)═C\C═C\C═C(/C)\C═C\C═C(/C)\C═C\C | beta-Bixin | HMDB |
| (O)═O | | |
| CC(═O)O\C1═C(C)\CCC(CC(O)C(═C)CC1)C(C)(C)O | Tanacetol B | HMDB |
| OC(═O)C1═CC(OS(O)(═O)═O)═CC═C1 | 3-hydroxybenzoic | HMDB |
| | acid-3-O-sulphate | |
| OC[C@H]1O[C@H](O[C@H]2[C@H](O)[C@@H](O)[C@@H](O[C@H]3[C | Maltohexadecaose | HMDB |
| @H](O)[C@@H](O)[C@@H](O[C@H]4[C@H](O)[C@@H](O)[C@@H](O | | |
| [C@H]5[C@H](O)[C@@H](O)[C@@H](O[C@H]6[C@H](O)[C@@H](O)[C | | |
| @@H](O[C@H]7[C@H](O)[C@@H](O)[C@@H](O[C@H]8[C@H](O)[C@ | | |
| @H](O)[C@@H](O[C@H]9[C@H](O)[C@@H](O)[C@@H](O[C@H]%10[C | | |
| @H](O)[C@@H](O)[C@@H](O[C@H]%11[C@H](O)[C@@H](O)[C@@H] | | |
| (O[C@H]%12[C@H](O)[C@@H](O)[C@@H](O[C@H]%13[C@H](O)[C@@ | | |
| H](O)[C@@H](O[C@H]%14[C@H](O)[C@@H](O)[C@@H](O[C@H]%15 | | |
| [C@H](O)[C@@H](O)[C@@H](O[C@H]%16[C@H](O)[C@@H](O)[C@@H] | | |
| (O)O[C@@H]%16CO)O[C@@H]%15CO)O[C@@H]%14CO)O[C@@H]% | | |
| 13CO)O[C@@H]%12CO)O[C@@H]%11CO)O[C@@H]%10CO)O[C@@H] | | |
| 9CO)O[C@@H]8CO)O[C@@H]7CO)O[C@@H]6CO)O[C@@H]5CO)O[C | | |
| @@H]4CO)O[C@@H]3CO)O[C@@H]2CO)[C@H](O)[C@@H](O)[C@@H] | | |
| 1O | | |
| CC(═O)OCC1OC(OC2═CC3═C(C═C2)C(═O)C(═CO3)C2═CC═C(O)C═C2) | 6″-O-Acetyldaidzin | HMDB |
| C(O)C(O)C1O | | |
| COC1═C(Cl)C(C)═C(C(═O)OC2CC3(C)C4CC(C)(C)CC4C(O)C(CO)═C23) | Arnamiol | HMDB |
| C(O)═C1 | | |
| CC(CC(O)C1OC2CCC3(CCC(O3)\C═C\C(C)C3CC(C)═CC4(OC(CC(C)(O) | Dinophysistoxin 1 | HMDB |
| C(O)═O)CCC4O)O3)OC2C(O)C1═C)C1OC2(CCC1C)OCCCC2C | | |
| CCCCCCCCC═CCCCCCCCC(═O)OCC(COP(O)(O)═O)OC(═O)CCCCCCC | 9-Octadecenoic | HMDB |
| C═CCCCCCCCC | acid 1- | |
| | [(phosphonoxy)methyl]- | |
| | 1,2- | |
| | ethanediyl ester | |
| OC[C@H]1O[C@H](O[C@H]2[C@H](O)[C@@H](O)[C@@H](O[C@H]3[C | Maltododecaose | HMDB |
| @H](O)[C@@H](O)[C@@H](O[C@H]4[C@H](O)[C@@H](O)[C@@H](O | | |
| [C@H]5[C@H](O)[C@@H](O)[C@@H](O[C@H]6[C@H](O)[C@@H](O)[C | | |
| @@H](O[C@H]7[C@H](O)[C@@H](O)[C@@H](O[C@H]8[C@H](O)[C@ | | |
| @H](O)[C@@H](O[C@H]9[C@H](O)[C@@H](O)[C@@H](O[C@H]%10[C | | |
| @H](O)[C@@H](O)[C@@H](O[C@H]%11[C@H](O)[C@@H](O)[C@@H] | | |
| (O[C@H]%12[C@H](O)[C@@H](O)[C@@H](O)O[C@@H]%12CO)O[C@ | | |
| @H]%11CO)O[C@@H]%10CO)O[C@@H]9CO)O[C@@H]8CO)O[C@@H] | | |
| 7CO)O[C@@H]6CO)O[C@@H]5CO)O[C@@H]4CO)O[C@@H]3CO)O[C | | |
| @@H]2CO)[C@H](O)[C@@H](O)[C@@H]1O | | |
| [H][C@@]12CC[C@H](OC(═O)CCCCCC)[C@@]1(C)CC[C@@]1([H])[C@ | Testosterone | HMDB |
| @]2([H])CCC2═CC(═O)CC[C@]12C | enanthate | |
| CC1═C(C)C(═O)OC(C1)C1(C)OC23CCC1(O)C2(C)CCC1C3CC═C2CC(CC | Coagulin R 3- | HMDB |
| (═O)C12C)OC1OC(CO)C(O)C(O)C1O | glucoside | |
| COC1═C(OC)C═C2C3C[C@@H](O)[C@@H](CC(C)C)CN3CCC2═C1 | alpha- | HMDB |
| | dihydrotetrabenazine | |
| O[C@@H]1[C@@H](O)[C@H](OC2═C(O)C═C(CC3CCC(═O)O3)C═C2O) | 5-(3′,4′,5′- | HMDB |
| O[C@@H]([C@H]1O)C(O)═O | Trihydroxyphenyl)- | |
| | gamma- | |
| | valerolactone-4′- | |
| | O-glucuronide | |
| OC(CCC(O)═O)CC1═CC═C(OS(O)(═O)═O)C═C1 | 4-Hydroxy-5-(4′- | HMDB |
| | hydroxyphenyl)- | |
| | valeric acid-4′-O- | |
| | sulphate | |
| OS(═O)(═O)ON═O | Nitrosylsulfuric | HMDB |
| | acid | |
| NCC(═O)N[C@@H](CCC(N)═O)C(═O)N1CCC[C@H]1C(═O)N[C@@H](CC | Rigin | HMDB |
| CNC(N)═N)C(O)═O | | |
| OC(CCCCC(O)═O)CC(O)═O | 3-Hydroxysuberic | HMDB |
| | acid | |
| CCC(NC(C)C(═O)N1[C@H]2CCCC[C@H]2C[C@H]1C(═O)O[C@@]1(O[C | Perindoprilat | HMDB |
| @H](C)[C@@H](O)[C@H](O)[C@H]1O)C(O)═O)C(O)═O | glucuronide | |
| CC1═C(N)C(C)═C(C(═C1N(═O)═O)C(C)(C)C)N(═O)═O | 4-amino-MX | HMDB |
| CC(N)COC1═C(C)C═CC═C1CO | 2- | HMDB |
| | hydroxymexiletine | |
| CC1CCCC(O)CCCCCC2═CC(O)═CC(O)═C2C(═O)O1 | Zeranol | HMDB |
| CSCC[C@H](NC(═O)[C@@H](N)CC1═CNC2═CC═CC═C12)C(═O)N[C@ | Tetragastrin | HMDB |
| @H](CC(O)═O)C(═O)N[C@@H](CC1═CC═CC═C1)C(N)═O | | |
| CCOC1═C(C═CC(CC(O)═NC(CC(C)C)C2═CC═CC═C2N2CCCC(O)C2)═C | hydroxyrepaglinide | HMDB |
| 1)C(O)═O | | |
| OC(CCC(═O)OS(O)(═O)═O)CC1═CC═CC═C1 | 4-Hydroxy-5- | HMDB |
| | (phenyl)-valeric | |
| | acid-O-sulphate | |
| OC1OCC2C1COC2C1═CC2═C(OCO2)C═C1 | Samin | HMDB |
| CC(═O)NC(CSC(═S)NCC═C)C(O)═O | N-acetyl-S-(N- | HMDB |
| | allylthiocarbamoyl)- | |
| | L-cysteine | |
| CCCC1═CC(═O)OC2═C3CC(OC3═C(C(═O)CC(C)C)C(O)═C12)C(C)(C)O | Furomammea G | HMDB |
| O[C@@H]1[C@@H](COP(O)(═O)OP(O)(═O)OP(O)(O)═O)O[C@H]([C@@ | 6-Thioinosine | HMDB |
| H]1O)N1C═NC2═C1N═CN═C2S | triphosphate | |
| CC1OC(CC(O)C1OC1OC(CO)C(O)C(O)C1O)OC1CCC2(C)C(CCC3C2CC | Coroloside | HMDB |
| C2(C)C(CCC32O)C2═CC(═O)OC2)C1 | | |
| CCCCC[C@H](O)\C═C\[C@H]1[C@H](O)CC(═O)[C@H]1CCCCCCC(O)═O | 8-Isoprostaglandin | HMDB |
| | E1 | |
| CN1C═NC═C1C[C@H](NC(═O)CCCN)C(O)═O | Homoanserine | HMDB |
| COC(═O)\C═C\C1═CC═C(OC2OC(CO)C(O)C(O)C2O)C═C1 | Linocinnamarin | HMDB |
| OCC1OC(C(O)C(O)C1O)C1C2═C(C(O)═CC═C2)C(═O)C2═C1C═C(CO)C═ | Aloin | HMDB |
| C2O | | |
| CC1═C(C)C(═O)OC(C1)C(C)(O)C1(O)CCC2(O)C3CC(O)C4═CC═CC(═O)C | Withaperuvin C | HMDB |
| 4(C)C3CCC12C | | |
| OC1═CC═CC2═C1C(═O)C1═C(C═CC═C1O)C2═O | Dantron | HMDB |
| OC1═CC2═C(C═C1O)C1═C(O2)C(═O)C2═C(OC3═C2C═C(O)C(O)═C3)C1═O | Thelephoric acid | HMDB |
| NCCCC[C@H](NC(═O)[C@@H]1CCCN1C(═O)[C@@H]1CCCN1)C(═O)N | Postin | HMDB |
| [C@@H](CCCNC(N)═N)C(O)═O | | |
| CC1OC(OCC(O)C(O)C(O)C(NC(C)═O)C═O)C(O)C(O)C1O | 2-Acetamido-2- | HMDB |
| | deoxy-6-O-a-L- | |
| | fucopyranosyl-D- | |
| | glucose | |
| C[N+]1(C)C═CC2═CC(O)═C([O−])C═C12 | N,N- | HMDB |
| | dimethylindoliumolate | |
| CCCCNCC(O)C1═CC(Cl)═CC2═C1C1═C(C═C(Cl)C═C1)\C2═C\C1═CC═C | desbutyl- | HMDB |
| (Cl)C═C1 | lumefantrine | |
| CC1OC(CC(O)C1O)OC1C(O)CC(OC2CCC3(C)C(CCC4C3CC(O)C3(C)C | Digoxigenin | HMDB |
| (CCC43O)C3═CC(═O)OC3)C2)OC1C | bisdigitoxoside | |
| CN1CCC23C4OC5═C(OC6OC(C(O)C(O)C6O)C(O)═O)C═CC(CC1C2CC[C | Dihydroisomorphine- | HMDB |
| @H]4O)═C35 | 3-glucuronide | |
| N[C@@H](CC1═CC═C(O)C═C1)C(═O)N[C@@H](CCCNC(N)═N)C(O)═O | Kyotorphin | HMDB |
| CCCCCCCCCCCCCCCCCC(═O)OC(CC([O−])═O)C[N+](C)(C)C | Stearoylcarnitine | HMDB |
| O[C@H]1[C@H](O)[C@@H](OC2═CC═CC═C2O)O[C@H]([C@@H]1O)C | Diphenol | HMDB |
| (O)═O | glucuronide | |
| CCN(CC)C(═S)S(C)═O | Methyl-N,N- | HMDB |
| | diethylthiocarbamate | |
| OCCCCC(O)═O | 5- | HMDB |
| | Hydroxypentanoic | |
| | acid | |
| COC1═CC═C(N2CN(C═O)C3═CC═C(OC)C═C3C2)C(CO)═C1 | Dictyoquinazol B | HMDB |
| OC1═CC═C(C═C1)[C@H]1COC2═C(C1)C═CC(O)═C2 | Equol | HMDB |
| CNC[C@H]1CCCC[C@]1(O)C1═CC(O)═CC═C1 | N,O- | HMDB |
| | Didesmethyltramadol | |
| CN1C(N)═NC2═C1C═CC1═C2N═CC═N1 | 3-Methyl-3H- | HMDB |
| | imidazo[4,5- | |
| | f]quinoxalin-2- | |
| | amine | |
| CC1CCC(\C═C(/C)C═O)C2═C(C)CCC12 | Valerenal | HMDB |
| OC(CCCC(O)═O)C(O)═O | 2-Hydroxyadipic | HMDB |
| | acid | |
| CC1═CC═CC═C1C1OCC(CO)O1 | [2-(2- | HMDB |
| | Methylphenyl)-1,3- | |
| | dioxolan-4- | |
| | yl]methanol | |
| CCCCCCCCCCCCCCCCCC(═O)SCCNC(═O)CCNC(═O)C(O)C(C)(C)COP | Stearoyl-CoA | HMDB |
| (O)(═O)OP(O)(═O)OC[C@H]1O[C@H]([C@H](O)[C@@H]1OP(O)(O)═O)N | | |
| 1C═NC2═C1N═CN═C2N | | |
| OC(═O)C1═NCCCC1 | D-1-Piperideine-2- | HMDB |
| | carboxylic acid | |
| OC(═O)[C@@H](OC(═O)\C═C\C1═CC(O)═C(O)C═C1)[C@H](OC(═O)\C═ | Chicoric acid | HMDB |
| C\C1═CC(O)═C(O)C═C1)C(O)═O | | |
| CC1CS(═O)CC(N1)C(O)═O | Cycloalliin | HMDB |
| OCC1OC(O[As](O)(O)═O)C(O)C1O | Ribose-1-arsenate | HMDB |
| O═C1NC2═CC═CC═C2O1 | 2-Benzoxazolol | HMDB |
| C\C(COC1OC(CO)C(O)C(O)C1O)═C/C#N | Osmaronin | HMDB |
| OC1═C(C═O)C═C(C═C1)C1(NC(═O)NC1═O)C1═CC═CC═C1 | Phenytoin | HMDB |
| | methylcatechol | |
| O═C1NC(═O)C2CC═CCC12 | 3alpha,4,7,7alpha- | HMDB |
| | Tetrahydro-1H- | |
| | isoindole-1,3(2H)- | |
| | dione | |
| CCCCCCCCC[C@H](O)CC(═O)SCCNC(═O)CCNC(═O)[C@H](O)C(C)(C) | (S)-3- | YMDB |
| COP(O)(═O)OP(O)(═O)OC[C@H]1O[C@H]([C@H](O)[C@@H]1OP(O)(O)═O) | hydroxylauroyl- | |
| N1C═NC2═C1N═CN═C2N | CoA | |
| COC1═CC2═C(C(O)═C1O)C(═O)C1═C(C═CC(C)═C1O)C2═O | \1,7,8-trihydroxy- | PMN |
| | 6-methoxy-2- | |
| | methylanthraquinone\ | |
| CC(C)C1═C2C(═O)C(C)CC(═O)C2═C(C═O)C([O−])═C1O | hemigossypolone | PMN |
| CC(C)C1═C(O)C(O)═C2COC3═CC(C)═CC1═C23 | desoxyhemigossypol | PMN |
| OCC1OC(OC2═CC3═C(C═C2O)[N+](═CC═C2CC([N+]C(═C2)C([O−])═O) | celosianin I | PMN |
| C([O−])═O)C(C3)C([O−])═O) | | |
| C(OC2OC(C(O)C(O)C2OC(═O)C═CC2═CC═C(O)C═C2)C([O−])═O) | | |
| C(O)C1O | | |
| [N+]C(CCONC(═[N+])NC(CC([O−])═O)C([O−])═O)C([O−])═O | canavaninosuccinate | PMN |
| COC1═C(OC)C(OC)═C2C(CCC(N(C)C═O)C3═CC(═O)C(OC)═CC═C23)═C1 | N- | PMN |
| | formyldemecolcine | |
| COC1═C(O)C2═C(C(═O)C3═C(C(O)═CC(C)═C3O)C2═O)C(OC)═C1OC | \1,4,5-trihydroxy- | PMN |
| | 6,7,8-trimethoxy- | |
| | 2- | |
| | methylanthraquinone\ | |
| CCC1═C(C)C2═CC3═C(C═C)C(C)═C4C═C5C(C)C(CCC(═O)OCC═C(C)CC | tetrahydrogeranyl | PMN |
| CC(C)CCCC(C)CCC═C(C)C)C6═[N+]5[Mg−−] | geranyl- | |
| 5(N34)N3C(═CC1═[N+]25)C(C)═C1C(═O)[C−](C(═O)OC)C6═C31 | chlorophyll a | |
| CC(C)═C1CCC2C(CCC3C(C)(CCCC23C)C(O)O)═C1 | neoabietadiene- | PMN |
| | diol | |
| COC1═C(OC)C(O)═C2C(═O)C([O−])═C(OC2═C1)C1═CC═C(O)C(O)═C1 | eupatolitin | PMN |
| OC1═CC2═C(C═C1O)[N+](═CC═C1CC(NC(═C1)C([O−])═O)C([O−])═O) | betanidin | PMN |
| C(C2)C([O−])═O | | |
| CCCCCC═CC═COOCCCCCCCC([O−])═O | colneleate | PMN |
| OCC1OC(CO)(OCC2(OC3OC(CO)C(O)C(O)C3O)OC(CO)C(O)C2O)C(O)C | 1-kestotriose | PMN |
| 1O | | |
| CCCCCCC(O)CC═CCCCCCCCC([O−])═O | ricinoleate | PMN |
| C[N+]1CCC23C4OC5═C(O)C═CC(CC1C2C═CC4═O)═C35 | morphinone | PMN |
| CCCCCCCCCCCCCCCC(═O)SCCNC(═O)CCNC(═O)C(O)C(C)(C)COP([O−]) | palmitoyl-CoA | PMN |
| (═O)OP([O−])(═O)OCC1OC(C(O)C1OP([O−])([O−])═O) | | |
| N1C═NC2═C1N═CN═C2N | | |
| [N+]CCCC(═O)C([O−])═O | 2-keto-ornithine | PMN |
| CCC═CCC═CCC═CCCCCCCCC(═O)OCC(COC1OC(CO)C(O)C(O)C1O)O | 1-18:3-2-16:3- | PMN |
| C(═O)CCCCCC═CCC═CCC═CCC | monogalactosyldiacyl | |
| | glycerol | |
| OC1═CC═C2C(═O)C(OC2═C1)C1OC11C═CC(═O)C═C1 | phenoxy radical | PMN |
| | VII | |
| CC12CC(O)CC3(OC1═O)C1CCC4(O)CC1(CC4═C)C(C23)C([O−])═O | gibberellin A29 | PMN |
| CCC1═C(CO)C2═CC3═C(C═C)C(C)═C4C═C5N═C(C(CCC(═O)OCC═C(C) | 71- | PMN |
| CCCC(C)CCCC(C)CCCC(C)C)C5C)C5═C6N([Mg]N34)C(═CC1═N2)C(C)═ | hydroxychlorophylla | |
| C6C(═O)[C−]5C(═O)OC | | |
| CCCCCCCCC1═C(CCCCCCCC([O−])═O)C1 | sterculate | PMN |
| COC1═CC2═C(C═C1OC)C1C(CO2)OC2═CC(O)═CC═C2C1═O | 9- | PMN |
| | demethylmunduserone | |
| COC1═C(OC2═CC([O−])═CC(O)═C2C1═O)C1═CC═C(O)C(O)═C1 | 3-O- | PMN |
| | methylquercetin | |
| CC(C)═CCNC1═NC═NC2═C1N═CN2C1OC(COP([O−])([O−])═O)C(O)C1O | N6-(delta2- | PMN |
| | isopentenyl)- | |
| | adenosine 5′- | |
| | monophosphate | |
| COC1═C(O)C═CC(C═CC(O)═CC(═O)C═CC2═CC═C(O)C═C2)═C1 | demethoxycurcumin | PMN |
| CCC(O)C1CCCC[N+]1 | conhydrine | PMN |
| COC1═C(OC)C═C(C═C1)C1C2C(COC2═O)C(O)C2═C(OC)C3═C(OCO3)C═ | 5′-demethoxy-6- | PMN |
| C12 | methoxypodophyllotoxin | |
| COC1C(O)C(O)C(O)C(O)C1O | D-pinitol | PMN |
| CCCC1OC(C)═C([O−])C1═O | 4-hydroxy-5- | PMN |
| | methyl-2-propyl- | |
| | 3(2H)-furanone | |
| COC(═O)C1═COC(OC2OC(CO)C(O)C(O)C2O)C(C═C)C1CC═O | secologanin | PMN |
| COC1═CC23CC[N+](C)C(CC4═CC(OC)═C(OC)C(OC)═C24)CC3═CC1═O | O- | PMN |
| | methylandrocymbine | |
| OCC1OC(OC2OC═C3C(CC4N(CC5═CC6═C(C═CC═C6)N═C45)C3═O)C2 | deoxypumiloside | PMN |
| C═C)C(O)C(O)C1O | | |
| COC1═CC(═CC═C1O)C(═O)OC1OC(CO)C(O)C(O)C1O | 1-O-vanilloyl-beta- | PMN |
| | D-glucose | |
| CSCCCCCC(SC1OC(CO)C(O)C(O)C1O)═NOS([O−])(═O)═O | 5- | PMN |
| | methylthiopentylglucosinolate | |
| CC(C)(COP([O−])(═O)OP([O−])(═O)OCC1OC(C(O)C1OP([O−])([O−])═O) | 3-hydroxybenzoyl- | PMN |
| N1C═NC2═C1N═CN═C2N)C(O)C(═O)NCCC(═O)NCCSC(═O)C1═CC═ | CoA | |
| CC(O)═C1 | | |
| OCC1OC(OC2═CC([O−])═ | apigeninidin 5-O- | PMN |
| CC3═C2C═CC(═[O+]3)C2═CC═C(O)C═C2)C(O)C(O)C1O | glucoside | |
| OCC1OC(OC2═CC(O)═CC(O)═C2C(═O)C═CC2═CC═C(O)C═C2)C(O)C(O) | chalcone 2′-O- | PMN |
| C1O | glucoside | |
| [N+]C(C[Se])C([O−])═O | L-selenocysteine | PMN |
| O[CH]C═C | allyl alcohol | PMN |
| [O−]C(═O)CC═CC([O−])═O | glutaconate | PMN |
| OC1═CC2═C(C═C1)C1OC3═C(C═C4OCOC4═C3)C1(O)CO2 | (+)-6a- | PMN |
| | hydroxymaackiain | |
| COC1═CC═C(C2COC3═CC(O)═CC═C3C2)C(O)═C1 | (−)-vestitol | PMN |
| CC(C)═CCC1═C2OC(CC(═O)C2═C(O)C═C1[O−])C1═C(O)C═C(O)C═C1 | leachianone G | PMN |
| COC1═C(O)C2═C(C═C1O)C(═O)C1═C(C(O)═CC═C1O)C2═O | robustaquinone F | PMN |
| COC1═NC2═C(NC(═O)N2C)C(═O)N1C | \O(2),1,9- | PMN |
| | trimethylurate\ | |
| COC1═CC2═C(C═C1OC)C1C(CO2)OC2═C(C═CC3═C2CC(O3)C(═C)CO) | dalcochinin | PMN |
| C1O | | |
| COC1═CC═C2N═CC═C(C(O)C3CC4CC[N+]3CC4C═C)C2═C1 | quinine | PMN |
| CCCC═C1OC(C)═C([O−])C1═O | (2E)-2-butylidene- | PMN |
| | 4-hydroxy-5- | |
| | methyl-3(2H)- | |
| | furanone | |
| [O−]C(═O)CC(═O)CC([O−])═O | acetonedicarboxylate | PMN |
| [O−]C1═CC(═O)C2═CC═CC═C2C1═O | lawsone | PMN |
| [N+]C(CN1C(═O)NC═CC1═O)C([O−])═O | L-isowillardiine | PMN |
| OC1═CC═C(C═C2OC3═CC(O)═CC═C3C2═O)C═C1 | hispidol | PMN |
| CC1═C(C═C)C(NC1═O)═CC1═C(C)C(CCC([O−])═O)═ | biliverdin-IX-alpha | PMN |
| C(N1)C═C1N═C(C═C2NC(═O)C(C═C)═C2C)C(C)═C1CCC([O−])═O | | |
| COC1═C(O)C(CC═C(C)CCC═C(C)CCC═C(C)CCC═C(C)CCC═C(C)CCC═ | 2-methoxy-6-(all- | PMN |
| C(C)CCC═C(C)CCC═C(C)CCC═C(C)C)═CC═C1 | trans- | |
| | nonaprenyl)phenol | |
| COC(═O)C═CC1═CC═CC═C1 | methylcinnamate | PMN |
| CN1c2cc(NS(═O)(═O)CCc3ccccc3)ccc2OCC(C)(C)C1═O | 2-phenyl-N-(3,3,5- | Nature |
| | trimethyl-4-oxo- | |
| | 2,3,4,5-tetrahydro- | |
| | 1,5-benzoxazepin- | |
| | 7-yl)ethane-1- | |
| | sulfonamide | |
| FC(F)(F)Oc1ccc(NC(═O)NC2CCN(CC2)C(═O)C2CC2)cc1 | 1-(1- | Nature |
| | cyclopropanecarbonyl | |
| | piperidin-4-yl)- | |
| | 3-[4- | |
| | (trifluoromethoxy) | |
| | phenyl]urea | |
| CCS(═O)(═O)Nc1ccc(cc1)C(═O)NCC1COc2ccccc2C1 | N-[(3,4-dihydro- | Nature |
| | 2H-1-benzopyran- | |
| | 3-yl)methyl]-4- | |
| | ethanesulfonamido | |
| | benzamide | |
| CN(Cc1cccc(NS(N)(═O)═O)c1)C(C)═O | N-methyl-N-{[3- | Nature |
| | (sulfamoylamino)phenyl] | |
| | methyl}acetamide | |
| CC1CCCN(C1)c1nc(C)cc(OCC(═O)Nc2c(C)cccc2C)n1 | N-(2,6- | Nature |
| | dimethylphenyl)-2- | |
| | {[6-methyl-2-(3- | |
| | methylpiperidin-1- | |
| | yl)pyrimidin-4- | |
| | yl]oxy}acetamide | |
| CCOc1ccc(cc1)N(CC)S(═O)(═O)N1CCCC(C1)C(═O)NCc1ccccc1OC | 1-[(4- | Nature |
| | ethoxyphenyl)(ethyl) | |
| | sulfamoyl]-N- | |
| | [(2- | |
| | methoxyphenyl)methyl] | |
| | piperidine-3- | |
| | carboxamide | |
| [H]C(═O)C1═C[C@@]2(C)C(CC[C@@]3([H])[C@]4([H])CC[C@](C)(O)[C@ | Formebolone | Nature |
| @]4(C)C[C@@H](O)[C@]23[H])═CC1═O | | |
| CC1(C)COc2ccc(NS(═O)(═O)Cc3ccc(Cl)cc3)cc2N(CC═C)C1═O | 1-(4- | Nature |
| | chlorophenyl)-N- | |
| | [3,3-dimethyl-4- | |
| | oxo-5-(prop-2-en- | |
| | 1-yl)-2,3,4,5- | |
| | tetrahydro-1,5- | |
| | benzoxazepin-7- | |
| | yl]methanesulfonamide | |
| COc1ccccc1C(═O)Nc1ccc2OCC(Cc2c1)C(═O)NCc1ccc(C)cc1 | 6-(2- | Nature |
| | methoxybenzamido)- | |
| | N-[(4- | |
| | methylphenyl)methyl]- | |
| | 3,4-dihydro- | |
| | 2H-1-benzopyran- | |
| | 3-carboxamide | |
| COc1cccc(C2CC(═NN2C(═O)C(C)(C)C)c2cccc(NS(C)(═O)═O)c2)c1O | N-{3-[1-(2,2- | Nature |
| | dimethylpropanoyl)- | |
| | 5-(2-hydroxy-3- | |
| | methoxyphenyl)- | |
| | 4,5-dihydro-1H- | |
| | pyrazol-3- | |
| | yl]phenyl}methane | |
| | sulfonamide | |
| Cc1ccc(F)cc1NC(═O)C1COc2ccccc2C1 | N-(5-fluoro-2- | Nature |
| | methylphenyl)-3,4- | |
| | dihydro-2H-1- | |
| | benzopyran-3- | |
| | carboxamide | |
| CC(C)CCN1c2ccc(NS(═O)(═O)Cc3cccc(C)c3)cc2OCC(C)(C)C1═O | N-[3,3-dimethyl-5- | Nature |
| | (3-methylbutyl)-4- | |
| | oxo-2,3,4,5- | |
| | tetrahydro-1,5- | |
| | benzoxazepin-8- | |
| | yl]-1-(3- | |
| | methylphenyl)methane | |
| | sulfonamide | |
| CC1═CC(═O)C(C)═CC1═O | \2,5-Dimethyl-1,4- | Nature |
| | benzoquinone\ | |
| Cc1ccccc1OCCCC(═O)NCC(C)(C)C(N)═O | N-(2-carbamoyl- | Nature |
| | 2,2-dimethylethyl)- | |
| | 4-(2- | |
| | methylphenoxy)butanamide | |
| CC(NC(═O)C1COc2ccccc2C1)c1ccc(Cl)cc1 | N-[1-(4- | Nature |
| | chlorophenyl)ethyl]- | |
| | 3,4-dihydro-2H- | |
| | 1-benzopyran-3- | |
| | carboxamide | |
| Cc1cccc(c1)C(═O)Nc1ccc2OCC(Cc2c1)C(═O)NC1CC1 | N-cyclopropyl-6- | Nature |
| | (3- | |
| | methylbenzamido)- | |
| | 3,4-dihydro-2H-1- | |
| | benzopyran-3- | |
| | carboxamide | |
| FC(F)(F)COc1ccc(OCC(F)(F)F)c(c1)C(═O)NC[C@H]1CCCCN1 | N-{[(2R)-piperidin- | Nature |
| | 2-yl]methyl}-2,5- | |
| | bis(2,2,2- | |
| | trifluoroethoxy)benzamide | |
| COc1cccc(C(═O)Nc2ccc3N(CC═C)C(═O)C(C)(C)COc3c2)c1OC | N-[3,3-dimethyl-4- | Nature |
| | oxo-5-(prop-2-en- | |
| | 1-yl)-2,3,4,5- | |
| | tetrahydro-1,5- | |
| | benzoxazepin-8- | |
| | yl]-2,3- | |
| | dimethoxybenzamide | |
| CCOc1ccccc1CCCNC(═O)c1ccc(C)c(NS(C)(═O)═O)c1 | N-[3-(2- | Nature |
| | ethoxyphenyl)propyl]- | |
| | 3- | |
| | methanesulfonamido- | |
| | 4- | |
| | methylbenzamide | |
| CC═C(C)C(═O)OC1CCN2CC═C(COC(═O)C(CO)═CC)C12 | Triangularine | Nature |
| CCN(CC)Cc1ccccc1NS(N)(═O)═O | N-{2- | Nature |
| | [(diethylamino)methyl] | |
| | phenyl}aminosulfonamide | |
| CN(C)S(═O)(═O)N1CCC(CC1)C(═O)NCCOc1ccccc1C | 1- | Nature |
| | (dimethylsulfamoyl)- | |
| | N-[2-(2- | |
| | methylphenoxy)ethyl] | |
| | piperidine-4- | |
| | carboxamide | |
| CNC(═O)COc1cccc(NS(N)(═O)═O)c1 | N-methyl-2-[3- | Nature |
| | (sulfamoylamino)phenoxy] | |
| | acetamide | |
| CN(C)S(═O)(═O)N1CCC(CC1)C(═O)NC1CC(C)(C)Oc2ccc(C)cc12 | 1- | Nature |
| | (dimethylsulfamoyl)- | |
| | N-(2,2,6- | |
| | trimethyl-3,4- | |
| | dihydro-2H-1- | |
| | benzopyran-4- | |
| | yl)piperidine-4- | |
| | carboxamide | |
| O═C(NCC1CCCCC1)C1COc2ccc(NC(═O)c3ccccc3)cc2C1 | 6-benzamido-N- | Nature |
| | (cyclohexylmethyl)- | |
| | 3,4-dihydro-2H-1- | |
| | benzopyran-3- | |
| | carboxamide | |
| C1[C@H]([C@H]([C@@H]([C@H]([C@@H]1O)O)O)O)O | | Nature |
| C1[C@H]([C@@H]([C@H]([C@H]([C@@H]1O)O)O)O)O | | Nature |
| C([C@@H]([C@H](CS)O)O)S | | Nature |
| C(CO)[C@@H]([C@@H](CO)O)O | | Nature |
| C(CBr)[P@](═O)([O−])O | | Nature |
| C(C[S@](═O)(═O)[O−])S | | Nature |
| C(CSCCO)O | | Nature |
| C1[C@@H]([C@H](O[C@H]1O)CO)O | | Nature |
| [C@@H]1([C@@H]2[C@@H]([C@@H]3[C@@H]([C@H]1O[C@H](O2)O3) | | Nature |
| O)O)O | | |
| C1[C@@H]([C@H](O[C@H]1O[P@](═O)([O−])[O−])CO)O | | Nature |
| C([C@@H](CS)S)O | | Nature |
| C([C@H](CS)S)O | | Nature |
| C([C@H]([C@H]([C@H]([C@@H]([C@@H](CO)O)O)O)O)O)O | | Nature |
| C([C@H]([C@H]([C@@H]([C@@H](CO)O)O)O)O)O | | Nature |
| C([C@@H]([C@@H]([C@H]([C@H](CO)O)O)O)O)O | | Nature |
| C([C@H]([C@H]([C@@H]([C@H](CO)O)O)O)O)O | | Nature |
| C([C@H]([C@H]([C@@H]([C@H]([C@@H](CO)O)O)O)O)O)O | | Nature |
| [C@H]1([C@H]([C@@H]([C@@H]([C@@H]([C@H]1O)O)O)O[P@](═O) | | Nature |
| ([O−])[O−])O)O | | |
| C(O)O[C@H]1[C@@H]([C@H]([C@H]([C@H]([C@@H]1O)O)O)O)O | | Nature |
| C([C@H]([C@H]([C@@H]([C@@H]([C@H](CO)O)O)O)O)O)O | | Nature |
| C([C@H]([C@@H]([C@H]([C@H](CO)O)O)O)O)O | | Nature |
| C([C@@H]([C@H]([C@@H]([C@H](CO)O)O)O)O)O | | Nature |
| C([C@H]([C@@H]([C@H]([C@@H](CO)O)O)O)O)O | | Nature |
| C([C@H]([C@@](CO)(CO)O)O)O | | Nature |
| C([C@H](CO)O)O | | Nature |
| C([C@H]([C@@H]([C@@H]([C@H](CO)O)O)O)O)O | | Nature |
| C([C@H]([C@H](CO)O)O)O | | Nature |
| C([C@@H]([C@H](CO)O)O)O | | Nature |
| C([C@H]([C@@H](CO)O)O)O | | Nature |
| C(CO)O | | Nature |
| C([C@@H]1[C@H]([C@H]([C@@H](O1)O[P@](═O)([O−])[O−])O)O)O | | Nature |
| C([C@@H]1[C@H]([C@@H]([C@H]([C@H](O1)O[P@](═O)([O−])[O−]) | | Nature |
| O)O)O)O | | |
| C([C@H]([C@@H]([C@H](CO)O)O)O)O | | Nature |
| C([C@@H]1[C@H]([C@H]([C@@H](O1)O)O)O)O | | Nature |
| C([C@@H]1[C@H]([C@@H]([C@H]([C@@H](O1)O)O)O)O)O | | Nature |
| | | Nature |

TABLE 3
SMILES STRUCTURES

CC(C)CC(=O)OCCC(C)CCCC(C)=C
CCCCCCCCCCCCC1CCC(=O)O1
NC1=NC=NC2=C1N=CN2[C@@H]1O[C@@H]2COP(O)(=O)O[C@H]2[C@H]1O
COC1=CC2=C(NC=C2CCNC(C)=O)C=C1
C\C(CCC=C)=C/CC[C@](C)(O)C=C
CCCC\C=C/CCOC(=O)CC
CN(C)C(=N)NCCC[C@H](N)C(O)=O
[H]OC1([H])C([H])([H])OC(=O)C([H])([H])C([H])([H])C1([H])O[H]
OC[C@@]1(O)CO[C@@H](OC[C@H]2O[C@@H](OC3=CC4=C(C(O)=C3)C(=O)C=
C(O4)C3=CC=C(O)C=C3)[C@H](O)[C@@H](O)[C@@H]2O)[C@@H]1O
[H]OC([H])(C(=O)N([H])C([H])([H])C([H])([H])C(=O)N([H])C([H])([H])C([H])([H])SC(=O)C([H])(C([H])
([H])[H])C([H])(O[H])C([H])([H])C([H])([H])C([H])(C([H])([H])[H])C1([H])C([H])([H])C([H])([H])C2
([H])C3([H])[C@]([H])(O[H])C([H])([H])[C@]4([H])C([H])([H])[C@]([H])(O[H])C([H])([H])C([H])([H])
C4(C([H])([H])[H])C3([H])C([H])([H])[C@]([H])(O[H])C12C([H])([H])[H])C(C([H])([H])[H])(C([H])
([H])[H])C([H])([H])OP(=O)(O[H])OP(=O)(O[H])OC([H])([H])[C@@]1([H])O[C@@]([H])(N2C([H])=NC
3=C2N=C([H])N=C3N([H])[H])[C@]([H])(O[H])[C@]1([H])OP(=O)(O[H])O[H]
[H]OC([H])(C(=O)N([H])C([H])([H])C([H])([H])C(=O)N([H])C([H])([H])C([H])([H])SC(=O)C([H])(C(=O)
C([H])([H])C([H])([H])C([H])(C([H])([H])[H])C1([H])C([H])([H])C([H])([H])[C@@]2([H])C3([H])[C@]
([H])(O[H])C([H])([H])[C@]4([H])C([H])([H])[C@]([H])(O[H])C([H])([H])C([H])([H])C4(C([H])([H])
[H])[C@@]3([H])C([H])([H])[C@]([H])(O[H])C12C([H])([H])[H])C([H])([H])[H])C(C([H])([H])[H])
(C([H])([H])[H])C([H])([H])OP(=O)(O[H])OP(=O)(O[H])OC([H])([H])[C@@]1([H])O[C@@]([H])
(N2C([H])=NC3=C2N=C([H])N=C3N([H])[H])[C@]([H])(O[H])[C@]1([H])OP(=O)(O[H])O[H]
[H]O[C@]1([H])C([H])([H])C([H])([H])[C@@]2(C([H])([H])[H])[C@@]([H])(C1([H])[H])C([H])([H])
[C@@]([H])(O[H])[C@@]1([H])[C@]3([H])C([H])([H])C([H])([H])[C@]([H])([C@]([H])(C([H])([H])[H])
C([H])([H])C([H])([H])C([H])([H])C([H])(C([H])([H])[H])C([H])([H])[H])[C@@]3(C([H])([H])[H])C
([H])([H])C([H])([H])[C@]21[H]
[H]O[C@]1([H])C([H])([H])C([H])([H])[C@@]2(C([H])([H])[H])C([H])(C1([H])[H])C([H])([H])[C@@]
([H])(O[H])C1([H])C3([H])C([H])([H])C([H])([H])C([H])(C([H])(C([H])([H])[H])C([H])([H])C([H])([H])
C([H])([H])C([H])(C([H])=O)C([H])([H])[H])[C@@]3(C([H])([H])[H])C([H])([H])C([H])([H])C21[H]
[H]OC(=O)C([H])([H])N([H])C(=O)C([H])(N([H])[H])C([H])([H])[H]
[H]OC([H])(C(=O)N([H])C([H])([H])C([H])([H])C(=O)N([H])C([H])([H])C([H])([H])SC(=O)C(=C([H])C
([H])([H])C([H])([H])C([H])(C([H])([H])[H])C1([H])C([H])([H])C([H])([H])C2([H])C3([H])[C@]([H])
(O[H])C([H])([H])[C@]4([H])C([H])([H])[C@]([H])(O[H])C([H])([H])C([H])([H])[C@]4(C([H])([H])[H])C3
([H])C([H])([H])C([H])([H])[C@]12C([H])([H])[H])C([H])([H])[H])C(C([H])([H])[H])(C([H])([H])[H])C
([H])([H])OP(=O)(O[H])OP(=O)(O[H])OC([H])([H])[C@@]1([H])O[C@@]([H])(N2C([H])=NC3=C2N=
C([H])N=C3N([H])[H])[C@]([H])(O[H])[C@]1([H])OP(=O)(O[H])O[H]
[H]OC([H])(C(=O)N([H])C([H])([H])C([H])([H])C(=O)N([H])C([H])([H])C([H])([H])SC(=O)C([H])(C(=O)
C([H])([H])C([H])([H])C([H])(C([H])([H])[H])C1([H])C([H])([H])C([H])([H])C2([H])C3([H])[C@]([H])
(O[H])C([H])([H])[C@]4([H])C([H])([H])[C@]([H])(O[H])C([H])([H])C([H])([H])[C@]4(C([H])([H])[H])
C3([H])C([H])([H])C([H])([H])[C@]12C([H])([H])[H])C([H])([H])[H])C(C([H])([H])[H])(C([H])([H])[H])
C([H])([H])OP(=O)(O[H])OP(=O)(O[H])OC([H])([H])[C@@]1([H])O[C@@]([H])(N2C([H])=NC3=C2
N=C([H])N=C3N([H])[H])[C@]([H])(O[H])[C@]1([H])OP(=O)(O[H])O[H]
[H]OC1([H])[C@@]([H])(O[C@]([H])(C([H])([H])OP(=O)(O[H])OP(=O)(O[H])OC([H])([H])[C@]([H])
(OC(=O)C([H])([H])C([H])([H])C([H])([H])C([H])([H])C([H])([H])C([H])([H])C([H])([H])C([H])=C([H])C
([H])([H])C([H])=C([H])C([H])([H])C([H])([H])C([H])([H])C([H])([H])C([H])([H])[H])C([H])([H])OC(=O)
C([H])([H])C([H])([H])C([H])([H])C([H])([H])C([H])([H])C([H])([H])C([H])([H])C([H])([H])C([H])([H])
C([H])([H])C([H])([H])C([H])([H])C([H])([H])C([H])([H])C([H])([H])[H])[C@]1([H])O[H])N1C([H])=C
([H])C(=NC1=O)N([H])[H]
[H]OC1([H])[C@@]([H])(O[C@]([H])(C([H])([H])OP(=O)(O[H])OP(=O)(O[H])OC([H])([H])[C@]([H])
(OC(=O)C([H])([H])C([H])([H])C([H])([H])C([H])=C([H])C([H])([H])C([H])=C([H])C([H])([H])C([H])=
C([H])C([H])([H])C([H])=([H])C([H])([H])C([H])([H])C([H])([H])C([H])([H])C([H])([H])[H])C([H])([H])O
C(=O)C([H])([H])C([H])([H])C([H])([H])C([H])([H])C([H])([H])C([H])([H])C([H])([H])C([H])([H])C([H])
([H])C([H])([H])C([H])([H])C([H])([H])C([H])([H])C([H])([H])C([H])([H])[H])[C@]1([H])O[H])N1C([H])=
C([H])C(=NC1=O)N([H])[H]
[H]OC1([H])[C@@]([H])(O[C@]([H])(C([H])([H])OP(=O)(O[H])OP(=O)(O[H])OC([H])([H])[C@]([H])
(OC(=O)C([H])([H])C([H])([H])C([H])([H])C([H])([H])C([H])([H])C([H])([H])C([H])=C([H])C([H])([H])C
([H])=C([H])C([H])([H])C([H])=C([H])C([H])([H])C([H])=C([H])C([H])([H])C([H])([H])[H])C([H])([H])O
C(=O)C([H])([H])C([H])([H])C([H])([H])C([H])([H])C([H])([H])C([H])([H])C([H])([H])C([H])([H])C([H])
([H])C([H])([H])C([H])([H])C([H])([H])C([H])([H])C([H])([H])C([H])([H])[H])[C@]1([H])O[H])N1C([H])=
C([H])C(=NC1=O)N([H])[H]
[H]OC1([H])[C@@]([H])(O[C@]([H])(C([H])([H])OP(=O)(O[H])OP(=O)(O[H])OC([H])([H])[C@]([H])
(OC(=O)C([H])([H])C([H])([H])C([H])([H])C([H])([H])C([H])([H])C([H])([H])C([H])([H])C([H])=C([H])C
([H])([H])C([H])=C([H])C([H])([H])C([H])([H])C([H])([H])C([H])([H])C([H])([H])[H])C([H])([H])OC(=O)
C([H])([H])C([H])([H])C([H])([H])C([H])([H])C([H])([H])C([H])([H])C([H])([H])C([H])([H])C([H])([H])
C([H])([H])C([H])([H])C([H])([H])C([H])([H])C([H])([H])C([H])([H])C([H])([H])C([H])([H])[H])[C@]1
([H])O[H])N1C([H])=C([H])C(=NC1=O)N([H])[H]
[H]OC1([H])[C@@]([H])(O[C@]([H])(C([H])([H])OP(=O)(O[H])OP(=O)(O[H])OC([H])([H])[C@]([H])
(OC(=O)C([H])([H])C([H])([H])C([H])([H])C([H])=C([H])C([H])([H])C([H])=C([H])C([H])([H])C([H])=C
([H])C([H])([H])C([H])=C([H])C([H])([H])C([H])([H])C([H])([H])C([H])([H])C([H])([H])[H])C([H])([H])O
C(=O)C([H])([H])C([H])([H])C([H])([H])C([H])([H])C([H])([H])C([H])([H])C([H])([H])C([H])([H])C([H])
([H])C([H])([H])C([H])([H])C([H])([H])C([H])([H])C([H])([H])C([H])([H])C([H])([H])C([H])([H])[H])
[C@]1([H])O[H])N1C([H])=C([H])C(=NC1=O)N([H])[H]
[H]OC(=O)C1=C([H])N=C(C(O[H])=C1C(=O)O[H])C([H])([H])[H]
[H]OC1([H])[C@@]([H])(O[C@]([H])(C([H])([H])OP(=O)(O[H])OP(=O)(O[H])OC([H])([H])[C@]([H])
(OC(=O)C([H])([H])C([H])([H])C([H])([H])C([H])([H])C([H])([H])C([H])([H])C([H])([H])C([H])([H])C
([H])=C([H])C([H])([H])C([H])=C([H])C([H])([H])C([H])=C([H])C([H])([H])C([H])([H])C([H])([H])C([H])
([H])C([H])([H])[H])C([H])([H])OC(=O)C([H])([H])C([H])([H])C([H])([H])C([H])([H])C([H])([H])C([H])
([H])C([H])([H])C([H])([H])C([H])([H])C([H])([H])C([H])([H])C([H])([H])C([H])([H])C([H])([H])C([H])
([H])[H])[C@]1([H])O[H])N1C([H])=C([H])C(=NC1=O)N([H])[H]
[H]OC1([H])[C@@]([H])(O[C@]([H])(C([H])([H])OP(=O)(O[H])OP(=O)(O[H])OC([H])([H])[C@]([H])
(OC(=O)C([H])([H])C([H])([H])C([H])=C([H])C([H])([H])C([H])=C([H])C([H])([H])C([H])=C([H])C([H])
([H])C([H])=C([H])C([H])([H])C([H])=C([H])C([H])([H])C([H])=C([H])C([H])([H])C([H])([H])[H])C([H])
([H])OC(=O)C([H])([H])C([H])([H])C([H])([H])C([H])([H])C([H])([H])C([H])([H])C([H])([H])C([H])([H])
C([H])([H])C([H])([H])C([H])([H])C([H])([H])C([H])([H])C([H])([H])C([H])([H])[H])[C@]1([H])O[H])N
1C([H])=C([H])C(=NC1=O)N([H])[H]
[H]OC1([H])[C@@]([H])(O[C@]([H])(C([H])([H])OP(=O)(O[H])OP(=O)(O[H])OC([H])([H])[C@]([H])
(OC(=O)C([H])([H])C([H])([H])C([H])([H])C([H])([H])C([H])([H])C([H])([H])C([H])=C([H])C([H])([H])C
([H])=C([H])C([H])([H])C([H])=C([H])C([H])([H])C([H])=C([H])C([H])([H])C([H])([H])[H])C([H])([H])O
C(=O)C([H])([H])C([H])([H])C([H])([H])C([H])([H])C([H])([H])C([H])([H])C([H])([H])C([H])([H])C([H])
([H])C([H])([H])C([H])([H])C([H])([H])C([H])([H])C([H])([H])C([H])([H])C([H])([H])C([H])([H])[H])
[C@]1([H])O[H])N1C([H])=C([H])C(=NC1=O)N([H])[H]
[H]OC1([H])[C@@]([H])(O[C@]([H])(C([H])([H])OP(=O)(O[H])OP(=O)(O[H])OC([H])([H])[C@]([H])
(OC(=O)C([H])([H])C([H])([H])C([H])([H])C([H])([H])C([H])([H])C([H])([H])C([H])([H])C([H])([H])C
([H])=C([H])C([H])([H])C([H])=C([H])C([H])([H])C([H])=C([H])C([H])([H])C([H])([H])C([H])([H])C([H])
([H])C([H])([H])[H])C([H])([H])OC(=O)C([H])([H])C([H])([H])C([H])([H])C([H])([H])C([H])([H])C([H])
([H])C([H])([H])C([H])([H])C([H])([H])C([H])([H])C([H])([H])C([H])([H])C([H])([H])C([H])([H])C([H])
([H])C([H])([H])C([H])([H])[H])[C@]1([H])O[H])N1C([H])=C([H])C(=NC1=O)N([H])[H]
COC1=C(CC(OO)C(C)=C)C(O)=C(C=C1)C(=O)\C=C\C1=CC=C(O)C=C1
COC1=CC=C(C(=O)\C=C\C2=CC=C(O)C=C2)C(O)=C1CC(O)C(C)=C
OCC(CO)OC=O
[H]OC(=O)C([H])([H])C([H])([H])C([H])([H])C(\[H])=C(\[H])C([H])([H])C(\[H])=C(/[H])\C(\[H])=
C(/[H])[C@@]([H])(O[H])C([H])([H])C([H])([H])C([H])([H])C([H])([H])C([H])([H])[H]
[H]OC([H])(C(=O)N([H])C([H])([H])C([H])([H])C(=O)N([H])C([H])([H])C([H])([H])SC(=O)C([H])=C
([H])C([H])=C([H])C([H])([H])C([H])=C([H])C([H])([H])C([H])=C([H])C([H])([H])C([H])=C([H])C([H])([H])
C([H])=C([H])C([H])([H])C([H])=C([H])C([H])([H])C([H])([H])[H])C(C([H])([H])[H])(C([H])([H])[H])C
([H])([H])OP(=O)(O[H])OP(=O)(O[H])OC([H])([H])[C@@]1([H])O[C@@]([H])(N2C([H])=NC3=C2N=
C([H])N=C3N([H])[H])[C@]([H])(O[H])[C@]1([H])OP(=O)(O[H])O[H]
[H]OC1([H])C([H])([H])C2(OC2(C([H])([H])C(=O)C(=C([H])C([H])=C([H])C(=C([H])C([H])=C([H])C
([H])=C(C([H])=C([H])C([H])=C(C([H])=C=C2C(O[H])(C([H])([H])[H])C([H])([H])C([H])(OC(=O)C([H])
([H])[H])C([H])([H])C2(C([H])([H])[H])C([H])([H])[H])C([H])([H])[H])C([H])([H])[H])C([H])([H])[H])C
([H])([H])[H])C(C([H])([H])[H])(C([H])([H])[H])C1([H])[H])C([H])([H])[H]
[H]OC1=NC(=NC(N([H])[H])=C1N(C([H])=O)C([H])([H])[H])N([H])[H]
CC(C)C1CCC(C)C=C1O
[H]OC([H])(C(=O)N([H])C([H])([H])C([H])([H])C(=O)N([H])C([H])([H])C([H])([H])SC(=O)C1=C([H])C
([H])([H])C([H])([H])C([H])=C1[H])C(C([H])([H])[H])(C([H])([H])[H])C([H])([H])OP(=O)(O[H])OP(=O)
(O[H])OC([H])([H])[C@@]1([H])O[C@@]([H])(N2C([H])=NC3=C2N=C([H])N=C3N([H])[H])[C@]
([H])(O[H])[C@]1([H])OP(=O)(O[H])O[H]
[H]OC([H])([H])[C@@]1([H])O[C@]([H])(OC([H])([H])C(=C([H])C([H])([H])N([H])C2=NC([H])=
NC3=C2N([H])C([H])=N3)C([H])([H])[H])[C@]([H])(O[H])[C@@]([H])(O[H])[C@]1([H])O[H]
OC(=O)CCCCCCC(=O)NCC(O)=O
N[C@@H](CC1=CC=C(O)C=C1)C(=O)N1CCC[C@H]1C(=O)N[C@@H](CC1=CNC2=CC=CC=C12)C
(=O)N[C@@H](CC1=CC=CC=C1)C(N)=O
OC1C2(Cl)C3(Cl)C4(Cl)C1(Cl)C1(Cl)C2(Cl)C3(Cl)C(Cl)(Cl)C41Cl
OC(=O)CCCNCCC(O)=O
[H][C@@]12CC[C@@](O)(C(=O)CO)[C@@]1(C)C[C@H](O)[C@@]1([H])[C@@]2([H])CCC2=CC
(=O)CC[C@]12C
OC[C@H]1O[C@@H](O[C@@H]2CO[C@@H](O)[C@H](O)[C@H]2O)[C@H](O)[C@@H](O)[C@H]
1O
CCCCC(C)C(=O)SCCNC(=O)CCNC(=O)C(O)C(C)(C)COP(O)(=O)OP(O)(=O)OC[C@H]1O[C@H]([C@H]
(O)[C@@H]1OP(O)(O)=O)N1C=NC2=C1N=CN=C2N
O[C@H]1[C@H](OP(O)(O)=O)[C@@H](OP(O)(O)=O)[C@@H](O)[C@@H](OP(O)(O)=O)[C@@H]
1OP(O)(O)=O
O[C@@H]1C=C[C@@H](NCC2=CNC3=C2C(=O)NC(=N)N3)[C@@H]1O
O[C@@H]1COC(=O)[C@H](O)[C@H]1O
CCCCCCCC\C=C/CC(=O)SCCNC(=O)CCNC(=O)[C@H](O)C(C)(C)COP(O)(=O)OP(O)(=O)OC[C@H]1
O[C@H](C(O)[C@H]1OP(O)(O)=O)N1C=NC2=C(N)N=CN=C12
CC(=O)C(=O)[C@H](N)[C@@H](O)[C@@H](OS(=O)(=O)O[C@@H]1[C@@H](COP(O)(=O)OP(O)
(O)=O)O[C@H]([C@@H]1O)N1C=CC(=O)NC1=O)[C@H](O)CO
OC1=NC2N=CN=CC2N1
CC(N)C(=O)NCC(O)=O
OC(=O)C(\O)=C\C1=CC=C(O)C=C1
COC1=C(O)C=C2[C@H](CC3=CC(O)=C(O)C=C3)N(C)CCC2=C1
C[C@@H](CCC=C(C)C)C1CCC2C3=C(CC[C@]12C)[C@@]1(C)CC[C@H](O)[C@](C)(C1CC3)C(O)=O
N[C@@H](CCCC(=O)OP(O)(=O)OCC1OC([C@H](O)[C@@H]1O)N1C=NC2=C(N)N=CN=C12)C(O)=
O
[H][C@@](COC(=O)CCCCCCCCCCCCCCC)(COP(O)(=O)OP(O)(=O)OC[C@H]1O[C@H](C(O)
[C@H]1O)N1C=CC(N)=NC1=O)OC(=O)CCCCCCC\C=C/C\C=C/CCCCC
[H][C@@](COC(=O)CCCCCCCCCCCCCCCCC)(COP(O)(=O)OP(O)(=O)OC[C@H]1O[C@H](C(O)
[C@H]1O)N1C=CC(N)=NC1=O)OC(=O)CCCCCCC\C=C/C\C=C/CCCCC
CC(C)CCCC(C)C1CCC2C3[C@@H](O)CC4CC(=O)CC[C@]4(C)C3CC[C@]12C
[H][C@@](COC(=O)CCCCCCCCCCCCCCC)(COP(O)(=O)OP(O)(=O)OC[C@H]1O[C@H]
(C(O)[C@H]1O)N1C=CC(N)=NC1=O)OC(=O)CCCCCCCCCCCCCCC
[H][C@@](COC(=O)CCCCCCCCCCCCCCCCC)(COP(O)(=O)OP(O)(=O)OC[C@H]1O[C@H](C(O)
[C@H]1O)N1C=CC(N)=NC1=O)OC(=O)CCCCCCCCCCCCCCC
OC(C=O)C(O)=O
CC1=NC=C(C=O)C(C(O)=O)=C1O
CC1=NC=C(C(O)=O)C(C(O)=O)=C1O
[H][C@@](COC(=O)CCCCCCCCCCCCCCC)(COP(O)(=O)OP(O)(=O)OC[C@H]1O[C@H]
(C(O)[C@H]1O)N1C=CC(N)=NC1=O)OC(=O)CCC\C=C/C\C=C/C\C=C/C\C=C/CCCCC
[H][C@@](COC(=O)CCCCCCCCCCCCCCC)(COP(O)(=O)OP(O)(=O)OC[C@H]1O[C@H]
(C(O)[C@H]1O)N1C=CC(N)=NC1=O)OC(=O)CCCCCC\C=C/C\C=C/C\C=C/C\C=C/CC
[H][C@@](COC(=O)CCCCCCCCCCCCCCC)(COP(O)(=O)OP(O)(=O)OC[C@H]1O[C@H]
(C(O)[C@H]1O)N1C=CC(N)=NC1=O)OC(=O)CCCCCCCC\C=C/C\C=C/C\C=C/CCCCC
[H][C@@](COC(=O)CCCCCCCCCCCCCCC)(COP(O)(=O)OP(O)(=O)OC[C@H]1O[C@H]
(C(O)[C@H]1O)N1C=CC(N)=NC1=O)OC(=O)CC\C=C/C\C=C/C\C=C/C\C=C/C\C=C/C\C=C/CC
[H][C@@](COC(=O)CCCCCCCCCCCCCCCCC)(COP(O)(=O)OP(O)(=O)OC[C@H]1O[C@H](C(O)
[C@H]1O)N1C=CC(N)=NC1=O)OC(=O)CCC\C=C/C\C=C/C\C=C/C\C=C/CCCCC
[H][C@@](COC(=O)CCCCCCCCCCCCCCCCC)(COP(O)(=O)OP(O)(=O)OC[C@H]1O[C@H](C(O)
[C@H]1O)N1C=CC(N)=NC1=O)OC(=O)CCCCCC\C=C/C\C=C/C\C=C/C\C=C/CC
[H][C@@](COC(=O)CCCCCCCCCCCCCCCCC)(COP(O)(=O)OP(O)(=O)OC[C@H]1O[C@H](C(O)
[C@H]1O)N1C=CC(N)=NC1=O)OC(=O)CCCCCCCC\C=C/C\C=C/C\C=C/CCCCC
OC(=O)C(\O)=C\C1=CC=C(O)C=C1
COC1=C(O)C=C2[C@H](CC3=CC(O)=C(O)C=C3)N(C)CCC2=C1
C[C@@H](CCC=C(C)C)C1CCC2C3=C(CC[C@]12C)[C@@]1(C)CC[C@H](O)[C@](C)(C1CC3)C(O)=O
CCCCCCCCCCCCCC(=O)SCCNC(=O)CCNC(=O)C(O)C(C)(C)COP(O)(=O)OP(O)(=O)OC[C@H]1O
[C@H]([C@H](O)[C@@H]1OP(O)(O)=O)N1C=NC2=C1N=CN=C2N
NC1=NC(=O)C2=C(NCC(CN(C=O)C3=CC=C(C=C3)C(=O)N[C@@H](CCC(O)=O)C(O)=O)N2)N1
O[C@@H]1[C@@H](COP(O)(=O)OP(O)(O)=O)OC([C@@H]1O)N1C=NC2=C1NC=NC2=O
NCC(=O)CCC(O)=O
OC[C@H]1O[C@H](O[C@H]2O[C@H](COP(O)(O)=O)[C@@H](O)[C@H](O)[C@H]2O)[C@H](O)
[C@@H](O)[C@@H]1O
CC[C@](O)(C(C)=O)C(O)=O
[H][C@@]1(CC=C2C3=C(CC[C@]12C)[C@@]1(C)CC[C@H](O)[C@@H](C)[C@]1([H])CC3)[C@H]
(C)CCC(=C)C(C)C
CC[C@](O)(C(C)=O)C(O)=O
CCCC(C(C)C)C(=O)OC1O[C@@H]([C@@H](O)[C@H](O)[C@H]1O)C(O)=O
[H]C(=O)C(O)C(O)C(OC1OC(CO)C(O)C(OC2OC(CO)C(OC3OC(CO)C(O)C(OC4OC(CO)C(OC5OC(C)
C(O)C(O)C5O)C(OC5OC(CO)C(O)C(O)C5O)C4NC(C)=O)C3O)C(OC3OC(C)C(O)C(O)C3O)C2NC(C)=O)
C1O)C(O)CO
OCC1OC(C(O)C(O)C1O)N1C=NC2=C1N=CN=C2NCC1=CC=CO1
CCCCCCCCC=CCCCCCCCC(=O)OCC(COP([O−])
(=O)OCC[N+](C)(C)C)OC(=O)CCCCCCCC=CCCCCCCCC
[N+]C(CO)CC1=CNC=N1
CC(C)=CCCC(C)=CC=O
COC1=C(O)C=CC(C=CC(=O)NCCC2=CC=C(O)C=C2)=C1
CCC=CCC1C(CCCCCCCC([O−])=O)C=CC1=O
[O−]C(=O)CCC1=C2CC3=C(CCC([O−])=O)C(CC([O−])=O)=C(CC4=C(CC([O−])=O)C
(CCC([O−])=O)=C(CC5=C(CC([O−])=O)C(CCC([O−])=O)=C(CC(N2)=C1CC([O−])=O)N5)N4)N3
OC1CC(O)(CC(=O)C1O)C([O−])=O
CC(C)C(C)C(O)C(O)C(C)C1CCC2C3CC(O)C4CC(O)C(O)CC4(C)C3CCC12C
CC(=O)C=CC1=C(C)CCCC1(C)C
CCCCCCCCCCCCCCCC(=O)OC(COC1OC(CO)C(O)C(O)C1O)COC(=O)CCCCCCCC=CCC=CCCCCC
CC(C)(COP([O−])(=O)OP([O−])(=O)OCC1OC(C(O)C1OP([O−])([O−])=O)
N1C=NC2=C1N=CN=C2N)C(O)C(=O)NCCC(=O)NCCSC(=O)C=CC1=CC=C(O)C=C1
CC(C)C(=C)CCC(C)C1CC=C2C3=C(CCC12C)C1(C)CCC(O)C(C)C1CC3
CC(=O)SCCNC(=O)CCNC(=O)C(O)C(C)(C)COP([O−])(=O)OP([O−])(=O)OCC1OC(C(O)C1OP([O−])
([O−])=O)N1C=NC2=C1N=CN=C2N
CC1(CC2C34OC3CC3C5(C)CCC(OC6OCC(OC7OC(CO)C(O)C(O)C7O)C(O)C6OC6OC(CO)C(O)C(O)
C6O)C(C)(CO)C5CCC3(C)C4(C)CC(O)C2(C)CC1OC(=O)C1=CC=CC=C1)C=O
[H][C@@]12CC[C@H]([C@H](C)CCCC(C)C)[C@@]1(C)CC[C@@]1([H])[C@@]2([H])CC=C2CC(CI)
CC[C@]12C
CCCC(=O)OC1CC[C@]2(C)C3CC[C@]4(C)[C@H](CCC4C3CC=C2C1)[C@H](C)CCCC(C)C
CC(=O)OC(C)(C)CCC(=O)C(C)(O)C1C(O)CC2(C)C3CC=C4C(CC(O)C(=O)C4(C)C)C3(C)C(=O)CC12C
Cn1/c(=N/C(=O)[C@@]([C@](F)(F)F)([C@](F)(F)F)Br)cc([O−])n(c1=O)C
Cn1/c(=N/C(=O)[C@]([C@](F)(F)F)([C@](F)(F)F)Cl)cc([O−])n(c1=O)C
CC[C@@]1(C(=N)N(C(=O)N(C1=O)C)C)CC
CC[C@@]1(C(=N)N(C(=O)N(C1=O)C)CC)CC
CCC[C@]1(C(=N)N(C(=O)N(C1=O)C)C)CCC
CCCN1C(=N)[C@](C(=O)N(C1=O)C)(CC)CC
CC[C@@]1(/C(=N/[C@@H](C)C)NC(=O)N(C1=O)C)CC
CCC[C@]1(C(=N)NC(=O)N(C1=O)C)CCC
CCCC[C@@]1(C(=N)N(C(=O)N(C1=O)C)C)CCCC
C[C@@H](C)CC[C@@]1(C(=[NH+])NC(=S)N=C1[O−])CC[C@H](C)C
CCC/N=C/1[C@](C(=O)N(C(=O)N1)C)(CC)CC
CCC[C@]1(/C(=N/C)NC(=O)N(C1=O)C)CCC
CCC[C@]1(C(=[NH+])NC(=S)N=C1[O−])CCC
CCCC/N=C/1[C@](C(=O)N(C(=O)N1)C)(CC)CC
CCCC[C@@]1(C(=N)NC(=O)N(C1=O)C)CCCC
CCCC[C@@]1(/C(=N/C)NC(=O)N(C1=O)C)CCCC
CCN(CC)/C(=N\[S@](=O)(=O)c1ccc(cc1)C)/C(=C\1N(/C(=[N+]/[C@](C)(C)C)S1)C)C
c1cn(c(=O)nc1N)[C@H]1[C@@H]([C@@H]([C@H](O1)CO[P@](=O)([O−])O[P@@](=O)([O−])
[O−])O)O
CC[C@@]1(C(=NC(=O)N(C1=O)C)Nc1ccc(cc1)Cl)CC
CC[C@@]1(/C(=N/c2ccccc2)N(C(=O)N(C1=O)C)C)CC
c1cn(c(=O)nc1N)[C@H]1C[C@@H]([C@H](O1)CO[P@](=O)([O−])O[P@@](=O)([O−])[O−])O
CC[C@@]1(C(=N)NC(=O)N(C1=O)C)c1ccccc1
CC[C@]1(C(=N)NC(=O)N(C1=O)C)c1ccccc1
c1cn(c(=O)nc1N)[C@@H]1[C@@H]([C@@H]([C@H](O1)CO)O)O
c1cn(c(=O)nc1N)[C@H]1[C@@H]([C@@H]([C@H](O1)CO)O)O
c1cn(c(=O)nc1N)[C@H]1[C@H]([C@@H]([C@H](O1)CO)O)O
c1[n+]c(nc(=O)n1[C@H]1[C@@H]([C@@H]([C@H](O1)CO)O)O)N
c1cn(c(=O)nc1N)[C@@H]1[C@H]([C@@H]([C@H](O1)CO)O)O
CC[C@@]1(/C(=N/Cc2ccc(cc2)OC)NC(=O)N(C1=O)C)CC
c1cn(c(=O)nc1N)[C@H]1[C@@H]([C@@H]([C@H](O1)CO[P@](=O)([O−])[O−])O)O
CC(=O)c1c(nc2n(c1=O)CCC2)N
CC[C@@]1(C(=NC(=O)NC1=O)Nc1cccc(c1)C)CC
CC[C@@]1(C(=NC(=O)NC1=O)Nc1ccccc1OC)CC
c1cn(c(=O)nc1N)[C@H]1C[C@@H]([C@H](O1)CO[P@](=O)([O−])[O−])O
CC/C(=N\c1cc([n+]c2c1cccc2)N(C)C)N(C)C
CCC[C@]1(C(=NC(=O)NC1=O)Nc1cccc(c1)C)CCC
c1cn(c(=O)nc1N)[C@@H]1C[C@H]([C@@H](O1)CO)O
c1cn(c(=O)nc1N)[C@@H]1C[C@@H]([C@@H](O1)CO)O
c1cn(c(=O)nc1N)[C@H]1C[C@@H]([C@@H](O1)CO)O
c1cn(c(=O)nc1N)[C@H]1C[C@H]([C@@H](O1)CO)O
c1cn(c(=O)nc1N)[C@H]1C[C@@H]([C@H](O1)CO)O
CCC[C@]1(C(=NC(=O)NC1=O)Nc1ccc(cc1)OC)CCC
C/N=C1\C/C(=[N+]/C)N([C@H](c2c1cccc2)O)C
C/N=C1\C/C(=[N+]/C)N([C@@H](c2c1cccc2)O)C
CCC[C@]1(C(=[N+]C(=O)NC1=O)N)CCC
CCC[C@]1(C(=NC(=O)NC1=O)Nc1ccccc1)CCC
CCCC[C@@]1(C(=NC(=O)NC1=O)Nc1ccccc1)CCCC
Cc1cc(c(cc1)/N=C/[N+](=C/Nc1c(cc(cc1)C)C)C)C
CN(C)c1cc2n(c(=O)n1)CCc1c2cc(c(c1)OC)OC
CCCC[C@@]1(C(=[N+]C(=O)NC1=O)N)CCCC
c1cn(c(=O)nc1N)[C@H]1[C@@H]([C@@H]([C@H](O1)CO[P@](=O)([O−])O[P@](=O)
([O−])O[P@](=O)([O−])[O−])O)O
c1cn(c(=O)nc1N)[C@H]1C[C@@H]([C@H](O1)CO[P@](=O)([O−])O[P@](=O)
([O−])O[P@](=O)([O−])[O−])O
C[N@@+](C)(C)CCO[P@](=O)([O−])O[P@@](=O)([O−])
OC[C@@H]1[C@H]([C@H]([C@@H](O1)n1ccc(nc1=O)N)O)O
CC[C@@H](C)Nc1cc2n(c(=O)n1)CCc1c2cc(c(c1)OC)OC
CC[C@H](C)Nc1cc2n(c(=O)n1)CCc1c2cc(c(c1)OC)OC
C[C@H](C)CNc1cc2n(c(=O)n1)CCc1c2cc(c(c1)OC)OC
COc1cc2c(cc1OC)c1cc(nc(=O)n1CC2)N(CCO)CCO
C[C@@H](C)CCCNc1cc2n(c(=O)n1)CCc1c2cc(c(c1)OC)OC
CCCCCC[C@@H](C)Nc1cc2n(c(=O)n1)CCc1c2cc(c(c1)OC)OC
CCCCCC[C@H](C)Nc1cc2n(c(=O)n1)CCc1c2cc(c(c1)OC)OC
CCCCN(CCCC)c1cc2n(c(=O)n1)CCc1c2cc(c(c1)OC)OC
CC(=O)NCCCCNc1cc2n(c(=O)n1)CCc1c2cc(c(c1)OC)OC
CCC(=O)NCCCCCNc1cc2n(c(=O)n1)CCc1c2cc(c(c1)OC)OC
C/C(=N\c1c2ccccc2[n+]c2c1CCN2C)N(C)C
CC(=O)N(c1ccc(cc1)OC)c1cc2n(c(=O)n1)CCc1c2cc(c(c1)OC)OC
CN(CC[C@H](CC(=O)N[C@H]1C=C[C@@H](O[C@@H]1C(=O)[O−])
n1ccc(nc1=O)N)[NH3+])C(=[NH2+])N
CN(CC[C@@H](CC(=O)N[C@H]1C=C[C@@H](O[C@@H]1C(=O)[O−])
n1ccc(nc1=O)N)[NH3+])C(=[NH2+])N
CN(CC[C@@H](CC(=O)N[C@@H]1C=C[C@@H](O[C@@H]1C(=O)[O−])
n1ccc(nc1=O)N)[NH3+])C(=[NH2+])N
CN(CC[C@H](CC(=O)N[C@@H]1C=C[C@@H](O[C@@H]1C(=O)[O−])
n1ccc(nc1=O)N)[NH3+])C(=[NH2+])N
CNc1cc2n(c(=O)n1)CCc1c2cc(c(c1)OC)OC
COc1cc(cc(c1OC)OC)CNc1cc2n(c(=O)n1)CCc1c2cc(c(c1)OC)OC
COc1cc2c(cc1OC)c1cc(nc(=O)n1CC2)NCCCO
COc1cc2c(cc1OC)c1cc(nc(=O)n1CC2)NCCCC[NH3+]
COCCCNc1cc2n(c(=O)n1)CCc1c2cc(c(c1)OC)OC
CCOCCCNc1cc2n(c(=O)n1)CCc1c2cc(c(c1)OC)OC
CCCCCCCNc1cc2n(c(=O)n1)CCc1c2cc(c(c1)OC)OC
COc1cc2c(cc1OC)c1cc(nc(=O)n1CC2)NCCCCCC[NH3+]
C[C@@H]1C[C@H](CN(C1)c1cc2n(c(=O)n1)CCc1c2cc(c(c1)OC)OC)C
C[C@H]1C[C@@H](CN(C1)c1cc2n(c(=O)n1)CCc1c2cc(c(c1)OC)OC)C
CC(C)(OC1OC(CO)C(O)C(O)C1O)C1CCC(CO)(O1)C=C
CC1=C(O)C(=N)CC1
NCCCCCNC(N)=N
CC(C)=CCC1=C2OC=C(C(=O)C2=C(O)C=C1O)C1=C(O)C=C(O)C=C1
COC1=C(O)C2=C(C=C1)C1COC3=C(C=CC(OC)=C3O)C1O2
CCCCCCCC1OC1CC#CC#CC(CC)OC(C)=O
COC1=CC(OC)=C(OC)C2=C1C(=O)C1=C(O2)C(OC)=C2OC(C)(C)C=CC2=C1O
CC(O)C1OC(=O)C=C1
CC(=O)C1=C2NC(=O)OC2=CC=C1
COC1=C(OC)C=C(C=C1)C1OCC2(O)C1COC2C1=CC(OC)=C(O)C=C1
COC1=CC=C(C=C1)C1=CC(=O)C2=C(O1)C(OC)=C(OC)C=C2O
CCCCCCCCCCCCCCCCCC(O)CCCCCCCCCCC
CC(CC(=O)\C=C(\C)C1CC(O)C2(C)C3=C(C(−O)CC12C)C1(C)CCC(=O)C(C)(C)C1CC3O)C(O)=O
COC1=CC(=O)C2=C(C=C(C)C(C(C)=O)=C2O)C1=O
CC(CC(=O)\C=C(\C)C1CC(O)C2(C)C3=C(C(=O)CC12C)C1(C)CCC(O)C(C)(C)C1CC3O)C(O)=O
CC(=C)C1CC2=C(OC3=C(C(O)=CC4=C3C=CC(C)(C)O4)C2=O)C2=C1C(O)=C1OC(C)(C)C=CC1=C2O
CC(OC1OC(COC(C)=O)C(OC2OC(CO)C(O)C(O)C2O)C(O)C1O)C1CC(O)C2C3CCC4=CC(=O)CCC4(C)C
3CCC12C
CC1=COC2=C1CC1C(=C)CCC(=O)C1(C)C2
CC(=C)C1CC2=CC(C(=O)CO)=C(O)C=C2O1
CC1=C(O)C(C)=C2OC(CC(=O)C2=C1O)C1=CC=CC=C1
COC1=CC=CC2=C1C(CNC(=S)SC)=CN2
COC1=C(CC=C(C)C)C2=C(C(O)=C1)C(=O)C1=C(O2)C(O)=C(O)C=C1
OCC(O)C1OC2C(OC(=O)C3=C2C(O)=C(O)C(O)=C3C2=C(O)C(O)=C3OC(=O)C4=CC(O)=C(O)C5=
C4C3=C2C(=O)O5)C1O
COC1=CC(CC\C=C\C(=O)CCC2=CC(OC)=C(O)C(OC)=C2)=CC=C1O
CC(CC(=O)C=C(C)C)C1CC(O)C(C)(O)C=C1
COC1=CC=C(OC\C=C/C#CC2=CC=C(O)C=C2)C=C1
C\C(CC\C=C(/C)CCC(OC1OC(CO)C(O)C(O)C1O)C(C)(C)O)=C/CCC(C)(OC1OC(CO)C(O)C(O)C1O)
C=C
CCCCCCCCCCCCCCCC\C=C\CC\C=C\CCCCCCCCCCC1=CC(C)OC1=O
CC(C)CCC1=CC2=C(N1)N=CC=C2
COC1=C(O)C=C(C=C1O)[C@H]1OC2=C(C[C@H]1O)C(O)=CC(O[C@@H]1O[C@@H]([C@@H](O)
[C@H](O)[C@H]1O)C(O)=O)=C2
COC1=CC2=C(C(=O)C=C(O2)C2=CC=C(O)C=C2)C(O)=C1C1OC(CO)C(O)C(O)C1OC1OC(COC(=O)\
C=C\C2=CC(OC)=C(O)C=C2)C(O)C(O)C1O
CN1CCCC1CC(=O)CC1CCC(CC(C)=O)N1C
OCC1OC(OC2C(O)C(CO)OC(OCC3OC(O)C(O)C(O)C3O)C2O)C(O)C(O)C1O
CCCCC\C=C\CC(O)C(O)C(=O)CCCCCCC(O)=O
OCC1OC(OCC2OC(OC3=C(OC4=CC(O)=CC(O)=C4C3=O)C3=CC=C(O)C=C3)C(OC3OC(CO)C(O)C(O)
C3O)C(O)C2O)C(O)C(O)C1O
CCCCCCCCCCCCCCC1OC1CC\C=C/CCCCCCCCCCC1=CC(C)OC1=O
COC1=C(O)C=CC(=C1)C1COC2=CC(O)=CC=C2C1
CC\C=C/C1=CC2=C(C=CC=C2)C(=O)O1
COC1=CC2=C(C(=O)CC(O2)C2=CC(O)=C(OC)C=C2)C(OC2OC(CO)C(O)C(O)C2O)=C1
OCC1OC(OC2=CC(O)=CC(O)=C2C(=O)CCC2=CC=CC=C2)C(O)C(O)C1O
COC1=CC(=CC(O)=C1OC)C1=CC(=O)C2=C(O1)C(OC)=C(OC)C=C2
CC\C=C/C[C@@H]1[C@H](CCCCCC(O)=O)CCC1=O
[H][C@]12CCC(=C)[C@@]1([H])[C@@]13OC(=O)C(CCO[C@@H]4O[C@H](CO)[C@@H](O)
[C@H](O)[C@H]4O)=C1C=C[C@]2(O)CO3
CC(=O)O[C@H]1[C@]2(C)CC[C@@H](C2)C1(C)C
COC1=CC=C(C=C1)C1COC2=C(C=CC(O)=C2)C1=O
CC(C)=CCC1=C(O)C=C2OC3=C(C(=O)C2=C1O)C(CCC(C)(C)O)−C(O)C(O)=C3
OC1=CC(O)=C2C(=O)C(COC2=C1)C1=CC(O)=C(O)C=C1
CC(=O)C1CCS1
COC1=CC=C(C=C1)C1=C(OC)C(=O)C2=C(OC)C(OC)=C(Cl)C(OC)=C2O1
CC(C1CC(OC(C)=O)C2(O)C3CC4OC44C(O)C=CC(=O)C4(C)C3CCC12C)C1CC(C)=C(C)C(=O)O1
COC1=C(O)C=CC(=C1)C1=[O+]C2=C(C=C1O[C@@H]1O[C@H](CO[C@@H]3OC(C)[C@H](OC(=O)\
C=C\C4=CC(O)=C(O)C=C4)[C@@H](O)[C@H]3O)[C@@H](O)[C@H](O)[C@H]1O)C(O[C@@H]1
O[C@H](CO)[C@@H](O)[C@H](O)[C@H]1O)=CC(O)=C2
CSC1=NC(CC(C)=O)C2=C(NC3=CC=CC=C23)S1
[H]OC([H])([H])C([H])(OC([H])([H])C([H])([H])C([H])([H])C([H])([H])C(\[H])=C(\[H])C([H])([H])
C(\[H])=C(\[H])C([H])([H])C(\[H])=C(\[H])C([H])([H])C(\[H])=C(\[H])C([H])([H])C([H])([H])C([H])([H])
C([H])([H])C([H])([H])[H])C([H])([H])O[H]
CCC1C2N3CCC22C(CC1CC3)N(C(C)=O)C1=C2C=CC=C1OC
CC\C=C/C1=CC2=C(C=CC=C2)C(=O)O1
OC(CCC(O)=O)CC1=CC=C(O)C(O)=C1
OCC1OC(OC2=C(OC3=C(C(O)=CC(O)=C3)C2=O)C2=CC(O)=C(O)C(O)=C2C2=C(C=C(O)C(O)=C2O)C
2OC3=CC(O)=CC(O)=C3CC2OC(=O)C2=CC(O)=C(O)C(O)=C2)C(O)C(O)C1O
COC1=CC2=C(C(=O)C=C(O2)C2=CC=C(O)C=C2)C(O)=C1C
CC1C2C(CC3C4CC=C5CC(CCC5(C)C4CCC23C)OC2OC(CO)C(OC3OC(C)C(O)C(O)C3O)C(O)C2OC2O
C(CO)C(O)C(O)C2O)OC11CCC(C)(COC2OC(CO)C(O)C(O)C2O)O1
COC1=CC(=CC(O)=C1OC)C(O)=O
CC\C=C/C\C=C/C\C=C/CCCCCCCC(=O)OCC(O)COC1OC(COC2OC(CO)C(O)C(O)C2O)C(O)C(O)C1O
C\C=C\S(=O)CC(NC(=O)CCC(N)C(O)=O)C(O)=O
CC1(C)CC(O)C2(C)CCC3(C)C(=CCC4C5(C)CCC(O)C(C)(C=O)C5CCC34C)C2C1
OC(C(O)=O)C(O)=O
COC1=CC(=CC=C1O)C1=C(OC2OC(COC3OC(C)C(O)C(O)C3O)C(O)C(O)C2OC2OCC(O)(CO)C2O)C
(=O)C2=C(O1)C=C(O)C=C2O
CC1(C)CCCC2(C)C(\C=C\C(=C)C(O)CO)C(C)(O)C(O)C(O)C12
COC1=CC(\C=C\C(=O)O[C@@]2(C[C@@H](O)[C@H](O)[C@@H](C2)OC(=O)\C=C\C2=CC(O)=C
(O)C=C2)C(O)=O)=CC=C1O
[H][C@@]12O[C@]3([H])C=C(C)C(=O)C[C@]3(COC(C)=O)C(C)([C@H](OC(C)=O)[C@H]1O)
C21CO1
CC(C)=C1CCC2(C)CCC=C(C)C2C1
COC1=C(\C=C/C(=O)C2=CC=C(O)C=C2)C=C(C(O)=C1)C(C)(C)C=C
COC1=C(C)C(O)=C2C(=O)C=C(C)OC2=C1
CCS(=O)CC(N)C(O)=O
NC1=CC2=NC3=CC=CC=C3OC2=CC1=O
CC(C)CCCC(C)CCCC(C)CCCC(C)(O)CCC1=CC(=O)C(C)=C(C)C1=O
ONC(=O)OCC1C2NC(=N)NC22N(CCC2(O)O)C(=N)N1O
COC1=CC=CC(OC)=C1C1=CC(=O)C2=C(O1)C=CC(OC)=C2OC
COC1=CC(OC)=C2C(OC3=C2C(=O)OC2=C3C=CC(O)=C2)=C1
CC(CCC1(O)OC2CC3C4CC(O)C5CC(OC6OC(CO)C(OC7OC(CO)C(O)C(OC8OC(CO)C(O)C(O)C8O)C7O)
C(O)C6O)C(O)CC5(C)C4CCC3(C)C2C1C)COC1OC(CO)C(O)C(O)C1O
CCCCCCCCCCCCCCCCCCC(O)CCCCCCCCCCCCC
CC(C)=CCC\C(C)=C\C=C\C(\C)=C\C=C\C(\C)=C\C=C/C=C(\C)/C=C/C=C(\C)/C=C/C=C(\C)CCC=C
(C)C
CC(=C)C1CCC2(C)CCC(=O)C(C)=C2C1
CCCCCC(=O)CCOC1OC(CO)C(O)C(O)C1O
COC1=CC=C(O)C2=C1CC(C(C)=C)C(C)(C2)C=C
CC(=O)C1=C2OC3=C(C(O)=CC4=C3C=CC(C)(C)O4)C(=O)C2=CC2=C1C(=O)OC2(C)C
CCCCCCCCCCC1OC(CCC1O)C(O)CCC(O)C1CCC(CCCCCCCC(O)CC2=CC(C)OC2=O)O1
CC1OC(OCC2OC(OC3=C(OC4=CC(O)=CC5=[O+]C(C)=CC3=C45)C3=CC(O)=C(O)C(O)=C3)C(O)
C(O)C2O)C(O)C(O)C1O
OC(=O)C1CSC(=S)C1
CC(C)C1=C2C3=CC=C(CC(OC4OCC(O)C(O)C4O)C3(C)CCC2(C)CC1)C=O
OCC1OC(OC2=CC−C(\C=C\C(=O)OCC3CCN4CCCC34)C=C2)C(O)C(O)C1O
COC(=O)NC1=NC2=C(N1)C=CC=C2
CCCCCC(O)CCCCC(O)C1CCC(O1)C1CCC(O1)C(O)CCCCCCCCCCC1=CC(C)OC1=O
COC1=C(C=CC(O)=C1)C1COC2=C(C1)C=CC(O)=C2
CC1C(C)C(=O)OC1C(O)C(C)(O)C1C(O)CC2C3CC4OC44C(O)C=CC(=O)C4(C)C3CCC12C
COC1=CC(=CC=C1O)[C@H]1COC2=CC(O)=CC=C2C1
O[C@H]1[C@H](OC2=C(O)C=C(\C=C\C(O)=O)C=C2)O[C@@H]([C@@H](O)[C@@H]1O)C(O)=O
CC1OC(CC(O)C1O)OC1CCC2(C=O)C3CCC4(C)C(CCC4(O)C3CCC2(O)C1)C1=CC(=O)OC1
CC1OC(OC2C(O)C(O)COC2OC(=O)C23CCC(C)(C)CC2C2=CCC4C5(C)CC(O)C(O)C(C)(C5CCC4(C)C2
(C)CC3)C(O)=O)C(O)C(O)C1OC1OCC(O)C(O)C1O
OC1COC(OC2C3C4=C(O)C=C(O)C=C4OC2(OC2=CC(O)=C4CC(O)C(OC4=C32)C2=CC=C(O)C(O)=C2)
C2=CC=C(O)C(O)=C2)C(O)C1O
COC1=C(O)C=CC(\C=C\C(=O)NCCC2=CNC3=C2C(=C(O)C=C3)C2=C3C(CCNC(=O)\C=C\C4=CC(OC)=C
(O)C=C4)=CNC3=CC=C2O)=C1
CC(=O)C1=C([O−])OC(C)=CC1=O
OC(=O)C(=O)CC1=CC=CC=C1
COC1=CC(\C=C\C(\O)=C\C(=O)\C=C\C2=CC(OC)=C(O)C=C2)=CC=C1O
CC1CCC(CC1)C(C)(C)OC(C)=O
CN1C2CCC1(CC(C2)OC(=O)CC(C)=S)C=O
CCOC(=O)C(C)(OCC)OCC
COC1=CC=C(C=C1)C(=O)OCC(O)=O
CC(=O)CCC1=CC=C(O)C=C1
CCCC[C@H]1OC(=O)C[C@H]1C
OC1=CC2=C(C(O)=C1)C(=O)C=C(O2)C1=CC(O)=C(O)C=C1
CCCCCC(O)CC(=O)CCC1CCC(O)C(C1)OC
COC1=CC(OC)=CC(O)=C1
CC1=C(Cl)C(O)=CC=C1NC1=C(C=CC=C1)C(O)=O
OS(=O)(=O)OC(=O)C1=CNC2=C1C=CC=C2
OC[C@H]1O[C@H](O[C@H]2[C@H](O)[C@@H](O)[C@@H](O[C@H]3[C@H](O)[C@@H](O)
[C@@H](O[C@H]4[C@H](O)[C@@H](O)[C@@H](O[C@H]5[C@H](O)[C@@H](O)[C@@H]
(O[C@H]6[C@H](O)[C@@H](O)[C@@H](O[C@H]7[C@H](O)[C@@H](O)[C@@H](O[C@H]8[C@H]
(O)[C@@H](O)[C@@H](O[C@H]9[C@H](O)[C@@H](O)[C@@H](O[C@H]%1O[C@H](O)[C@@H]
(O)[C@@H](O[C@H]%11[C@H](O)[C@@H](O)[C@@H](O[C@H]%12[C@H](O)[C@@H](O)[C@@H]
(O[C@H]%13[C@H](O)[C@@H](O)[C@@H](O)O[C@@H]%13CO)O[C@@H]%12CO)O[C(5)@
H]%11CO)O[C@@H]%1OCO)O[C@@H]9CO)O[C@@H]8CO)O[C@@H]7CO)O[C@@H]6CO)O
[C@@H]5CO)O[C@@H]4CO)O[C@@H]3CO)O[C@@H]2CO)[C@H](O)[C@@H](O)[C@@H]1O
C[C@@H](O)[C@H](N)C(=O)NC(CCCCN)C(=O)N1CCC[C@H]1C(=O)N[C@@H](CCCNC(N)=N)C
(O)=O
NS(=O)(=O)C1=C(Cl)C=C(NCC2=CC=CS2)C(=C1)C1=NNN=N1
COC(=O)\C=C\C(\C)=C\C=C\C(\C)=C\C=C\C=C(/C)\C=C\C=C(/C)\C=C\C(O)=O
CC(=O)O\C1=C(C)\CCC(CC(O)C(=C)CC1)C(C)(C)O
OC(=O)C1=CC(OS(O)(=O)=O)=CC=C1
OC[C@H]1O[C@H](O[C@H]2[C@H](O)[C@@H](O)[C@@H](O[C@H]3[C@H](O)[C@@H](O)
[C@@H](O[C@H]4[C@H](O)[C@@H](O)[C@@H](O[C@H]5[C@H](O)[C@@H](O)[C@@H]
(O[C@H]6[C@H](O)[C@@H](O)[C@@H](O[C@H]7[C@H](O)[C@@H](O)[C@@H](O[C@H]8[C@H](O)
[C@@H](O)[C@@H](O[C@H]9[C@H](O)[C@@H](O)[C@@H](O[C@H]%1O[C@H](O)[C@@H](O)
[C@@H](O[C@H]%11[C@H](O)[C@@H](O)[C@@H](O[C@H]%12[C@H](O)[C@@H](O)[C@@H]
(O[C@H]%13[C@H](O)[C@@H](O)[C@@H](O[C@H]%14[C@H](O)[C@@H](O)[C@@H]
(O[C@H]%15[C@H](O)[C@@H](O)[C@@H](O[C@H]%16[C@H](O)[C@@H](O)[C@@H](O)O
[C@@H]%16CO)O[C@@H]%15CO)O[C@@H]%14CO)O[C@@H]%13CO)O[C@@H]%12CO)O[C@@H]
%11CO)O[C@@H]%1OCO)O[C@@H]9CO)O[C@@H]8CO)O[C@@H]7CO)O[C@@H]6CO)O
[C@@H]5CO)O[C@@H]4CO)O[C@@H]3CO)O[C@@H]2CO)[C@H](O)[C@@H](O)[C@@H]1O
CC(=O)OCC1OC(OC2=CC3=C(C=C2)C(=O)C(=CO3)C2=CC=C(O)C=C2)C(O)C(O)C1O
COC1−C(CI)C(C)=C(C(=O)OC2CC3(C)C4CC(C)(C)CC4C(O)C(CO)=C23)C(O)=C1
CC(CC(O)C1OC2CCC3(CCC(O3)\C=C\C(C)C3CC(C)=CC4(OC(CC(C)(O)C(O)=O)CCC4O)O3)OC2C(O)
C1=C)C1OC2(CCC1C)OCCCC2C
CCCCCCCCC=CCCCCCCCC(=O)OCC(COP(O)(O)=O)OC(=O)CCCCCCCC=CCCCCCCCC
OC[C@H]1O[C@H](O[C@H]2[C@H](O)[C@@H](O)[C@@H](O[C@H]3[C@H](O)[C@@H](O)
[C@@H](O[C@H]4[C@H](O)[C@@H](O)[C@@H](O[C@H]5[C@H](O)[C@@H](O)[C@@H]
(O[C@H]6[C@H](O)[C@@H](O)[C@@H](O[C@H]7[C@H](O)[C@@H](O)[C@@H](O[C@H]8[C@H]
(O)[C@@H](O)[C@@H](O[C@H]9[C@H](O)[C@@H](O)[C@@H](O[C@H]%1O[C@H](O)[C@@H]
(O[C@@H](O[C@H]%11[C@H](O)[C@@H](O)[C@@H](O[C@H]%12[C@H](O)[C@@H](O)[C@@)H]
(O)O[C@@H]%12CO)O[C@@H]%11CO)O[C@@H]%1OCO)O[C@@H]9CO)O[C@@H]8CO)O
[C@@H]7CO)O[C@@H]6CO)O[C@@H]5CO)O[C@@H]4CO)O[C@@H]3CO)O[C@@H]2CO)[C@H]
(O)[C@@H](O)[C@@H]1O
[H][C@@]12CC[C@H](OC(=O)CCCCCC)[C@@]1(C)CC[C@@]1([H])[C@@]2([H])CCC2=CC(=O)CC
[C@]12C
CC1=C(C)C(=O)OC(C1)C1(C)OC23CCC1(O)C2(C)CCC1C3CC=C2CC(CC(=O)C12C)OC1OC(CO)C(O)
C(O)C1O
COC1=C(OC)C=C2C3C[C@@H](O)[C@@H](CC(C)C)CN3CCC2=C1
O[C@@H]1[C@@H](O)[C@H](OC2=C(O)C=C(CC3CCC(=O)O3)C=C2O)O[C@@H]([C@H]1O)
C(O)=O
OC(CCC(O)=O)CC1=CC=C(OS(O)(=O)=O)C=C1
OS(=O)(=O)ON=O
NCC(=O)N[C@@H](CCC(N)=O)C(=O)N1CCC[C@H]1C(=O)N[C@@H](CCCNC(N)=N)C(O)=O
OC(CCCCC(O)=O)CC(O)=O
CCC(NC(C)C(=O)N1[C@H]2CCCC[C@H]2C[C@H]1C(=O)O[C@@]1(O[C@H](C)[C@@H](O)[C@H]
(O)[C@H]1O)C(O)=O)C(O)=O
CC1=C(N)C(C)=C(C(=C1N(=O)=O)C(C)(C)C)N(=O)=O
CC(N)COC1=C(C)C=CC=C1CO
COC1=CC2=C(C(O)=C1O)C(=O)C1=C(C=CC(C)=C1O)C2=O
CC(C)C1=C2C(=O)C(C)CC(=O)C2=C(C=O)C([O−])=C1O
CC(C)C1=C(O)C(O)=C2COC3=CC(C)=CC1=C23
OCC1OC(OC2=CC3=C(C=C2O)[N+](=CC=C2CC([N+]C(=C2)C([O−])=O)C([O−])=O)C(C3)C
([O−])=O)C(OC2OC(C(O)C(O)C2OC(=O)C=CC2=CC=C(O)C=C2)C([O−])=O)C(O)C1O
[N+]C(CCONC(=[N+])NC(CC([O−])=O)C([O−])=O)C([O−])=O
COC1=C(OC)C(OC)=C2C(CCC(N(C)C=O)C3=CC(=O)C(OC)=CC=C23)=C1
COC1=C(O)C2=C(C(=O)C3=C(C(O)=CC(C)=C3O)C2−O)C(OC)=C1OC
CCC1=C(C)C2=CC3=C(C=C)C(C)=C4C=C5C(C)C(CCC(=O)OCC=C(C)CCCC(C)CCCC(C)CCC=C(C)C)C6=
[N+]5[Mg--]5(N34)N3C(=CC1=[N+]25)C(C)=C1C(=O)[C−](C(=O)OC)C6=C31
CC(C)=C1CCC2C(CCC3C(C)(CCCC23C)C(O)O)=C1
COC1=C(OC)C(O)=C2C(=O)C([O−])=C(OC2=C1)C1=CC=C(O)C(O)=C1
OC1=CC2=C(C=C1O)[N+](=CC=C1CC(NC(=C1)C([O−])=O)C([O−])=O)C(C2)C([O−])=O
CCCCCC=CC=COC=CCCCCCCC([O−])=O
OCC1OC(CO)(OCC2(OC3OC(CO)C(O)C(O)C3O)OC(CO)C(O)C2O)C(O)C1O
CCCCCCC(O)CC=CCCCCCCCC([O−])=O
C[N+]1CCC23C4OC5=C(O)C=CC(CC1C2C=CC4=O)=C35
CCCCCCCCCCCCCCCC(=O)SCCNC(=O)CCNC(=O)C(O)C(C)(C)COP([O−])(=O)OP
([O−])(=O)OCC1OC(C(O)C1OP([O−])([O−])=O)N1C=NC2=C1N=CN=C2N

We claim:

1. A composition comprising an isolated sweet taste modulator compound selected from the compounds in Table 1, and derivatives thereof, wherein said composition further comprises at least one sweetener.

2. An ingestible composition comprising a composition of claim 1 and an acceptable carrier for ingestion.

3. The ingestible composition of claim 1, wherein the sweetener is selected from natural caloric sweeteners, natural high-potency sweeteners, and synthetic sweeteners.

4. The ingestible composition of claim 2, which has an enhanced sweet taste as compared to an ingestible composition that does not have a compound from Table 1, or a derivative thereof, but is otherwise the same.

5. The ingestible composition of claim 2, wherein the ingestible composition is a food, beverage, pharmaceutical, nutraceutical, tobacco, or oral hygiene/cosmetic product.

6. A flavoring concentrate formulation comprising a composition of claim 1 and a carrier.
